(19) United States
(12) Patent Application Publication
(10) Pub. No.: US 2023/0320995 A1
(43) Pub. Date: Oct. 12, 2023

(54) IONIZABLE CATIONIC LIPIDS AND LIPID NANOPARTICLES

(71) Applicant: CAPSTAN THERAPEUTICS, INC., San Diego, CA (US)

(72) Inventors: Priya Prakash KARMALI, San Diego, CA (US); Steven TANIS, Carlsbad, CA (US)

(73) Assignee: CAPSTAN THERAPEUTICS, INC., San Diego, CA (US)

(21) Appl. No.: 18/296,363

(22) Filed: Apr. 5, 2023

Related U.S. Application Data
(60) Provisional application No. 63/362,501, filed on Apr. 5, 2022.
(60) Provisional application No. 63/366,462, filed on Jun. 15, 2022.
(60) Provisional application No. 63/489,381, filed on Mar. 9, 2023.

(51) Int. Cl. A61K 9/51; C07D 295/088; C07D 211/46; C07C 271/16; C07C 275/14; A61K 31/7105

(52) U.S. Cl. CPC A61K 9/5123; C07D 295/088; C07D 211/46; C07C 271/16; C07C 275/14; A61K 31/7105

(57) ABSTRACT

Ionizable cationic lipids, methods for synthesizing them, as well as intermediates useful in synthesis of these lipids and methods of synthesizing the intermediates are disclosed. The ionizable cationic lipids are useful as a component of lipid nanoparticles (LNP), which in turn can be used for the delivery of nucleic acids into cells in vivo or ex vivo. LNP compositions are also disclosed, including LNP comprising a functionalized lipid to enable conjugation of a binding moiety, and targeted LNP (tLNP), that is a LNP in which a binding moiety has been conjugated to the functionalized lipid and can serve as a targeting moiety to direct the tLNP to a desired tissue or cell type.

### RELATED APPLICATIONS

This application claims priority to U.S. Provisional Patent Application Nos. 63/489,381 filed Mar. 9, 2023, 63/366,462 filed Jun. 15, 2022, and 63/362,501 filed on Apr. 5, 2022, the entire contents of which are each herein incorporated by reference.

### BACKGROUND

Lipid formulations have been used in the laboratory for the delivery of nucleic acids into cells. Early formulations based on the cationic lipid 1,2-dioleoyl-3-trimethylammonium propane (DOTAP) and the ionizable, fusogenic lipid dioleoylphosphatidyl ethanolamine (DOPE) had a large particle size and were problematic when used in vivo, exhibiting too rapid clearance, tropism for the lung, and toxicity. Lipid nanoparticles (LNPs) comprising ionizable cationic lipids have been developed to address these issues to the extent that RNA based products, such as the siRNA ONPATTRO® and two mRNA-based SARS-CoV-2 vaccines have received regulatory approval and entered the market. There is limited ability to control which tissues or cells take up the LNP once administered. LNP administered intravenously are taken up primarily in the liver, lung, or spleen depending to a significant degree on net charge and particle size. It is possible to direct >90% of LNP to the liver by a combination of formulation and intravenous administration. Intramuscular administration can provide a clinically useful level of local delivery and expression. LNP can be redirected to other tissues or cell types by conjugating a binding moiety with specificity for the target tissue or cell type, for example, conjugating a polypeptide containing an antigen binding domain from an antibody, to the LNP. Nonetheless, avoiding uptake by the liver remains a challenge. Moreover, with current systems only a minor portion of the encapsulated nucleic acid is successfully delivered to the cells of interest and into the cytoplasm. Current formulations may release only 2-5% of the administered RNA into the cytoplasm (see for example Gilleron et al., Nat. Biotechnol. 31:638-646, 2013, and Munson et al., Commun. Biol. 4:211-224, 2021). Thus, there are remaining issues of off-target delivery, poor efficiency of release of nucleic acid into the cytoplasm, and toxicity associated with accumulation of the component lipids.

Therefore, this disclosure provides ionizable lipids and lipid nanoparticles to satisfy an urgent need in the field.

### SUMMARY

Certain aspects of the disclosure relate to an ionizable cationic lipid having a structure selected from the group consisting of Formula 1, Formula 2, and Formula 3.

Other aspects of the disclosure relate to a lipid nanoparticle (LNP), comprising one or more ionizable cationic lipids respectively and independently having a structure selected from the group consisting of Formula 1, Formula 2, and Formula 3. In certain embodiments, the LNP may further comprise one or more of a phospholipid, a sterol, a co-lipid, a PEG-lipid, or combinations thereof. Examples of the phospholipids includes, without limitation, dioleoylphosphatidyl ethanolamine (DOPE), dimyristoylphosphatidyl choline (DMPC), distearoylphosphatidylcholine (DSPC), dimyristoylphosphatidyl glycerol (DMPG), dipalmitoyl phosphatidylcholine (DPPC), 1,2-diarachidoyl-sn-glycero-3-phosphocholine (DAPC), and combinations thereof. Examples of the sterol include, without limitation, cholesterol, campesterol, sitosterol, stigmasterol, and combinations thereof. Examples of the co-lipid include, without limitation, cholesterol hemisuccinate (CHEMS), and a quaternary ammonium headgroup containing lipid. Examples of the quaternary ammonium headgroup containing lipid include, without limitation, 1,2-dioleoyl-3-trimethylammonium propane (DOTAP), N-(1-(2,3-dioleyloxy)propyl)-N,N,N-trimethylammonium (DOTMA), 3β-(N—(N′,N′-Dimethylaminoethane)carbamoyl)cholesterol (DC-Chol), and combinations thereof. Examples of PEG-lipid may comprise a PEG moiety of 1000-5000 Da molecular weight (MW), and/or fatty acids with a fatty acid chain length of C14-C18. Examples of the PEG-lipid include, without limitation, DMG-PEG2000 (1,2-dimyristoyl-rglycero-3-methoxypolyethylene glycol-2000), DPG-PEG2000 (1,2-dipalmitoyl-glycero-3-methoxypolyethylene glycol-2000), DSG-PEG2000 (1,2-distearoyl-glycero-3-methoxypolyethylene glycol-2000), DOG-PEG2000 (1,2-dioleoyl-glycero-3-methoxypolyethylene glycol-2000), DMPE-PEG200 (1,2-dimyristoyl-glycero-3-phosphoethanolamine-3-methoxypolyethylene glycol-2000), DPPE-PEG2000 (1,2-dipalmitoyl-glycero-3-phosphoethanolamine-3-methoxypolyethylene glycol-2000), DSPE-PEG2000 (1,2-distearoyl-glycero-3-phosphoethanolamine-3-methoxypolyethylene glycol-2000), DOPE-PEG2000 (1,2-dioleoyl-glycero-3-phosphoethanolamine-3-methoxypolyethylene glycol-2000), and combinations thereof. In certain embodiments, the PEG-lipid comprises an optically pure glycerol moiety. In certain embodiments, the LNP further comprises a functionalized PEG-lipid. In certain embodiments, the LNP of Embodiment 28, wherein the functionalized PEG-lipid has been conjugated with a binding moiety (e.g., an antibody). In certain embodiments, the functionalized PEG-lipid comprises fatty acids with a fatty acid chain length of C16-C18. In certain embodiments, the functionalized PEG-lipid comprise a dipalmitoyl lipid or a distearoyl lipid.

In certain embodiments, the LNP comprises 40 to 60 mol % ionizable cationic lipid. In certain embodiments, the LNP comprises 7 to 30 mol % phospholipid. In certain embodiments, the LNP comprises 20 to 45 mol % sterol. In certain embodiments, the LNP comprises 1 to 30 mol % co-lipid. In certain embodiments, the LNP comprises 0 to 5 mol % PEG-lipid. In certain embodiments, the LNP comprises 0.1 to 5 mol % functionalized PEG-lipid.

In certain embodiments, the LNP further comprises a nucleic acid (e.g., mRNA). In certain embodiments, the weight ratio of total lipid to nucleic acid is 10:1 to 50:1.

Other aspects of the disclosure relate to a method of delivering a nucleic acid into a cell comprising contacting the cell with one or more LNP's disclosed herein, wherein at least some of the LNP's comprise the nucleic acid.

### BRIEF DESCRIPTION OF THE DRAWINGS

FIGS. 1A-1F depict a synthetic scheme for compounds having a structure of Formula 1. FIG. 1A shows the synthesis starting with readily available reagents through intermediate I-fA. FIG. 1B shows the synthetic path from intermediate I-fA to Compounds having a structure of Formula 1, Y═O, NH or N—CH₃. FIG. 1C shows the synthetic path from intermediate I-fA to Compounds having a structure of Formula 1, Y═CH₂. Unless specified otherwise, all substituents are defined the same as Formula 1. Specifically, FIG. 1D shows the synthesis of intermediates I-c, I-d, I-e, and I-f, which are embodiments of Formulas I-cA, I-dA, I-eA, and I-fA, respectively, wherein p is 1, n is 1, and R is C₉ alkyl straight chain. FIG. 1E shows the synthetic path from intermediate I-f to Compounds A-1 to A-3, which are respectively embodiments of Formula 1 with Y=O, NH, and N—CH₃, wherein p is 1, n is 1, X is N(Me)₂, and R is C₉ alkyl straight chain. FIG. 1F shows the synthetic path from intermediate I-f to Compound A-4, which is an embodiment of Formula 1 with Y═CH₂, wherein p is 1, n is 1, X is N(Me)₂, and R is C₉ alkyl straight chain.

FIGS. 2A-2F depict a synthetic scheme for compounds having a structure of Formula 2. FIG. 2A shows the synthesis starting with readily available reagents through intermediate II-hA. FIG. 2B shows the synthetic path from intermediate II-hA to Compounds having a structure of Formula 2, Y═O, NH or N—CH₃. FIG. 2F shows the synthetic path from intermediate II-hA to Compounds having a structure of Formula 2, Y═CH₂. Unless specified otherwise, all substituents are defined the same as Formula 2. Specifically, FIG. 2D shows the synthesis of intermediates II-c, II-d, II-e, II-f, II-g, and II-h, which are embodiments of Formulas II-cA, II-dA, II-eA, II-fA, II-gA, and II-hA, respectively, wherein p is 1, n is 1, and R is C₉ alkyl straight chain when applicable. FIG. 2E shows the synthetic path from intermediate II-h to Compounds A-5 to A-7, which are respectively embodiments of Formula 2 with Y═O, NH, and N—CH₃, wherein p is 1, n is 1, and R is C₉ alkyl straight chain. FIG. 2F shows the synthetic path from intermediate II-g to Compound A-8, which is an embodiment of Formula 2 with Y═CH₂, wherein p is 1, n is 1, and R is C₉ alkyl straight chain.

FIGS. 3A-3D depict a synthetic scheme for compounds having a structure of Formula 3. FIG. 3A shows the synthesis starting with readily available reagents to Compounds having a structure of Formula 3, W═C═O. FIG. 3D shows the synthetic path from intermediate III-cA to Compounds having a structure of Formula 3, W═CH₂. Unless specified otherwise, all substituents are defined the same as Formula 3. FIG. 3C shows the synthesis starting with readily available reagents to intermediates III-a, III-b, III-c, and Compound A-9, which are embodiments of Formulas III-aA, III-bA, III-cA, and Formula 3, respectively, wherein p is 1, n is 2, and R_{c} is C₉ alkyl straight chain when applicable. FIG. 3D shows the synthetic path from intermediate III-c to intermediates III-d, III-e, III-f, and Compound A-10, which are embodiments of III-dA, III-eA, III-fA, and Formula 3 with W═CH₂, wherein p is 1, n is 2, and R_{c} is C₉ alkyl straight chain.

FIGS. 4A-B depict synthetic schemes for reagents that may be used to make polyethylene glycol-containing lipid head groups. FIG. 4A depicts the synthesis of an embodiment of XR125 in which m is 2 and o is 3 (see Table 3). FIG. 4B depicts the synthesis of an embodiment of XR126 in which o is 3 (see Table 3).

FIGS. 5A-C depict the viability (5A), frequency of transfection (5B), and level of expression as geometric mean fluorescence intensity (gMFI) of the transfected cells (5C) for HEK293F cells transfected with mCherry mRNA encapsulated in LNP in which the ionizable cationic lipid was one of Compounds A-2, A-11, A-12, A-13, A-14, or A-15.

FIG. 6 depicts the frequency and level of expression, as determined by flow cytometry, of mCherry mRNA transfected in vitro into mouse splenic T cells by CD5-targeted lipid nanoparticles incorporating the indicated ionizable cationic lipids A-2, A-11, A-12, A-13, A-14, and A-15, respectively. Expression level is presented as the mean fluorescence intensity (MFI; geometric mean) of the positive peak in the flow cytometry histogram and transfection rate is the proportion of CD3⁺ cells in the positive peak. *: 10a, 10f, and 10p are described in Journal of Medicinal Chemistry 63:12992-13012, 2020.

FIG. 7 depicts the frequency and level of expression, as determined by flow cytometry, of mCherry mRNA transfected in vivo into mouse splenic T cells by CD5-targeted lipid nanoparticles incorporating the indicated ionizable cationic lipids A-2, A-11, A-12, A-13, A-14, and A-15, respectively. Expression level is presented as the mean fluorescence intensity (MFI; geometric mean) of the positive peak in the flow cytometry histogram and transfection rate is the proportion of CD3⁺ cells in the positive peak. *:10a, 10f, and 10p are described in Journal of Medicinal Chemistry 63:12992-13012, 2020.

FIG. 8 depicts a conceptual biodegradation scheme for Compound A-11 (above the line) and the starting compound and end products of biodegradation (below the line). The disclosed ionizable cationic lipids, and particularly Compounds of Formula 1, may undergo biodegradation according to such a conceptual scheme, without being bound to any particular theory.

### DETAILED DESCRIPTION

The instant disclosure provides ionizable cationic lipids, methods for synthesizing them, as well as intermediates useful in synthesis of these lipids and methods of synthesizing the intermediates. The instant disclosure provides ionizable cationic lipids of this disclosure as a component of lipid nanoparticles (LNPs), which LNPs can be used for the delivery of nucleic acids into cells in vivo or ex vivo. LNP compositions are also disclosed herein, including LNPs comprising a functionalized PEG-lipid to enable conjugation of a binding moiety to generate targeted LNPs (tLNPs), that is LNPs containing a binding moiety that directs the tLNP to a desired tissue or cell type. Also disclosed herein are methods of delivering a nucleic acid into a cell comprising contacting the cell with a LNP or tLNP of this disclosure.

Prior to setting forth this disclosure in more detail, it may be helpful to provide abbreviations and definitions of certain terms to be used herein. Additional definitions are set forth throughout this disclosure.

### Abbreviations

Abbreviations used herein include:
BF₃-OEt₂—Boron trifluoride diethyl etherate
BOC—tert-Butyloxycarbonyl
CDI—carbonyl diimidazole
c Log D—calculated Log D
c Log P—calculated Log P (partition coefficient)
c-pKa—calculated pKa
DMF—Dimethylformamide
DMAP—4-Dimethylaminopyridine
EDC-HCl—1-Ethyl-3-(3′-dimethylaminopropyl)carbodiimide HCl
Et₃N—Triethylamine
MeOH—Methanol
MeOTf—methyl trifluoromethanesulfonate
Pd/C—Palladium on carbon
PEG—Polyethylene glycol
PPTs—Pyridinium p-toluenesulfonate
TFA—Trifluoroacetic acid

### Definitions

As used in the specification and claims, the singular form “a,” “an,” and “the” includes plural references unless the context clearly dictates otherwise. It should be understood that the terms “a” and “an” as used herein refer to “one or more” of the enumerated components.

The use of the alternative (e.g., “or”) should be understood to mean either one, both, or any combination thereof of the alternatives.

The term “about” as used herein in the context of a number refers to a range centered on that number and spanning 15% less than that number and 15% more than that number. The term “about” used in the context of a range refers to an extended range spanning 15% less than that the lowest number listed in the range and 15% more than the greatest number listed in the range.

Throughout this disclosure, any concentration range, percentage range, ratio range, or integer range is to be understood to include the value of any integer within the recited range and, when appropriate, fractions thereof (such as one tenth and one hundredth of an integer), unless otherwise indicated. Also, any number range of this disclosure relating to any physical feature, such as polymer subunits, size, or thickness, are to be understood to include any integer within the recited range, unless otherwise indicated. Throughout this disclosure, numerical ranges are inclusive of their recited endpoints, unless specifically stated otherwise.

Unless the context requires otherwise, throughout the present specification and claims, the word “comprise” and variations thereof, such as, “comprises” and “comprising” are to be construed in an open, inclusive sense, that is, as “including, but not limited to.” As used herein, the terms “include” and “comprise” are used synonymously.

The phrase “at least one of” when followed by a list of items or elements refers to an open-ended set of one or more of the elements in the list, which may, but does not necessarily, include more than one of the elements.

“Derivative,” as used herein, refers to a chemically or biologically modified version of a compound that is structurally similar to a parent compound and (actually or theoretically) derivable from that parent compound. Generally, a “derivative” differs from an “analogue” in that a parent compound may be the starting material to generate a “derivative,” whereas the parent compound may not necessarily be used as the starting material to generate an “analogue.” A derivative may have different chemical or physical properties than the parent compound. For example, a derivative may be more hydrophilic or hydrophobic, or it may have altered reactivity as compared to the parent compound.

Alkyl refers to a saturated hydrocarbon moiety, that is an alkane lacking one hydrogen leaving a bond that connects to another portion of an organic molecule. In some embodiments, hydrogens are unsubstituted. In other embodiments, one or more hydrogens of the alkyl group may be substituted with the same or different substituents.

Alkenyl refers to a hydrocarbon moiety with one or more carbon-carbon double bonds but that is otherwise saturated. In some embodiments, hydrogens are unsubstituted. In other embodiments, one or more hydrogens of the alkenyl group may be substituted with the same or different substituents.

Alkynoic refers to a carboxylic acid moiety comprising one or more carbon-carbon triple bonds. In some embodiments, hydrogens are unsubstituted. In other embodiments, one or more hydrogens of the alkynoic group may be substituted with the same or different substituents.

Amide refers to a carboxylic acid derivative comprising a carbonyl group of a carboxylic acid bonded to an amine moiety.

Aryl refers to an aromatic or heteroaromatic ring lacking one hydrogen leaving a bond that connects to another portion of an organic molecule. Examples of aryl include, without limitation, phenyl, naphthalenyl, pyridine, pyrimidine, pyrazine, pyrrole, furan, thiophene, imidazole, thiazole, oxazole, and the like.

Aryl-alkyl refers to a moiety comprising one or more aryl rings and one or more alkyl moieties. The position of the one or more aryl rings can vary within the alkyl portion of the moiety. For example, the one or more aryl rings may be at an end of the one or more alkyl moieties, be fused into the carbon chain of the one or more alkyl moieties, or substitute one or more hydrogens of one or more alkyl moieties; and the one or more alkyl moieties may substitute one or more hydrogens of the one or more aryl rings. In some embodiments, there is a single ring; while in other embodiments, that are multiple rings.

Branched alkyl is a saturated alkyl moiety wherein the alkyl group is not a straight chain. Alkyl portions such as methyl, ethyl, propyl, butyl, and the like, can be appended to variable positions of the main alkyl chain. In some embodiments, there is a single branch; while in other embodiments, there are multiple branches.

Branched alkenyl refers to an alkenyl group comprising at least one branch off the main chain which may be formed by substituting one or more hydrogens of the main chain with the same or different alkyl groups, e.g., without limitation, methyl, ethyl, propyl, butyl, and the like. In some embodiments, a branched alkenyl is a single branch structure, while in other embodiments, a branched alkenyl may have multiple branches.

Straight chain alkyl is a non-branched, non-cyclic version of the alkyl moiety described above.

Straight chain alkenyl is a non-branched, non-cyclic version of the alkenyl moiety described above.

Cycloalkyl refers to a moiety which is a cycloalkyl ring of 3-12 carbons. In some embodiments, a cycloalkyl is a single ring structure; while in other embodiments, a cycloalkyl may have multiple rings.

Cycloalkyl-alkyl refers to a moiety which contains one or more cycloalkyl rings of 3-12 carbons, and one or more alkyl moieties. The position of the cycloalkyl ring can vary within the alkyl portion of the moiety. For example, the one or more cycloalkyl rings may be at an end of the one or more alkyl moieties, be fused into the carbon chain of the one or more alkyl moieties, or substitutes one or more hydrogens of one or more alkyl moieties; and the one or more alkyl moieties may substitute one or more hydrogens of the one or more cycloalkyl rings. In some embodiments, the cycloalkyl ring is a single ring structure; while in other embodiments, a cycloalkyl-alkyl may have multiple rings.

Ester refers to a carboxylic acid derivative comprising a carbonyl group bond to an alkyloxy group to form an ester bond —C(═O)—O—.

Ether refers to an oxygen atom attached to 2 carbon-based moieties that are the same or different.

Head group refers to the hydrophilic or polar portion of a lipid.

Imide refers to a moiety comprising a nitrogen bond to two carbonyl groups.

Sterol refers to a subgroup of steroids that contain at least one hydroxyl (OH) group. Examples of sterols include, without limitation, cholesterol, ergosterol, R-sitosterol, stigmasterol, stigmastanol, 20-hydroxycholesterol, 22-hydroxycholesterol, and the like.

### Ionizable Cationic Lipids

Ionizable cationic lipids useful as a component of lipid nanoparticles for the delivery of nucleic acids, including DNA, mRNA, or siRNA into cells are disclosed. The ionizable cationic lipids have a c-pKa from 8 to 11 and c Log D from 9 to 18 or 11-14. These ranges can lead to a measured pKa in the LNP or tLNP of 6 to 7 facilitating ionization in the endosome. In some embodiments, somewhat greater basicity may be desirable and can be obtained from ionizable cationic lipids with c-pKa and c Log D in the stated ranges. In some embodiments, the c-pKa is about 8, about 9, about 10, or about 11, or in a range bound by any pair of these values. In some embodiments c Log D is about 9, about 10, about 11, about 12, about 13, about 14, about 15, about 16, about 17, about 18, or in a range bound by any pair of these values.

In certain aspects, an ionizable cationic lipid has a structure of Formula 1,

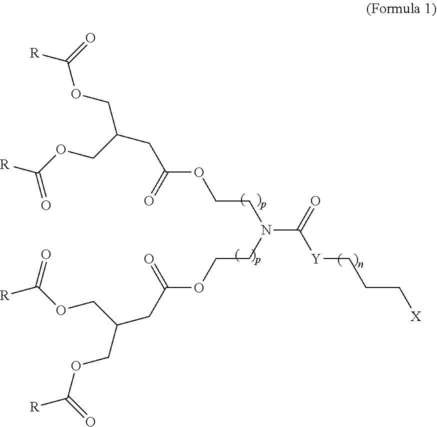

wherein Y is O, NH, N—CH₃, or CH₂,
n is an integer from 0 to 4,
   X is

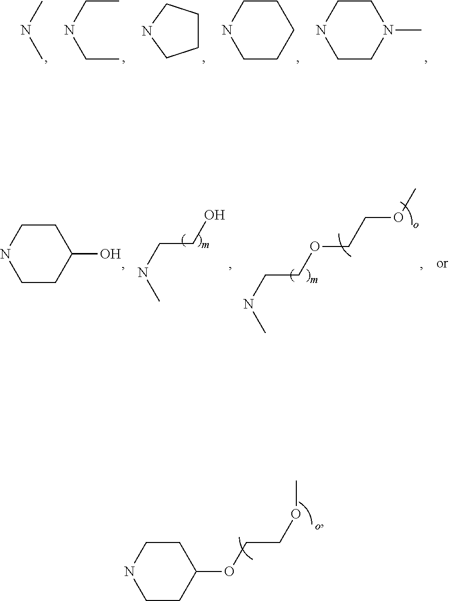

m is an integer from 1 to 3,
is an integer from 1 to 4,
p is an integer from 1 to 4,
wherein when p=1, each R is independently C₆ to C₁₆ straight-chain alkyl; C₆ to C₁₆ branched alkyl; C₆ to C₁₆ straight-chain alkenyl; C₆ to C₁₆ branched alkenyl; C₉ to C₁₆ cycloalkyl-alkyl in which the cycloalkyl is C₃ to C₈ cycloalkyl positioned at either end or within the alkyl chain; or C₈ to C₁₈ aryl-alkyl in which the aryl is phenyl or naphthalenyl and is positioned at either end or within the alkyl chain,
wherein when p=2, each R is independently C₆ to C₁₄ straight-chain alkyl; C₆ to C₁₄ straight-chain alkenyl; C₆ to C₁₄ branched alkyl; C₆ to C₁₄ branched alkenyl; C₉ to C₁₄ cycloalkyl-alkyl in which the cycloalkyl is C₃ to C₈ cycloalkyl positioned at the either end or within the alkyl chain; or C₈ to C₁₆ aryl-alkyl in which the aryl is phenyl or naphthalenyl and is positioned at either end or within the alkyl chain,
wherein when p=3, each R is independently C₆ to C₁₂ straight-chain alkyl; C₆ to C₁₂ straight-chain alkenyl; C₆ to C₁₂ branched alkyl; C₆ to C₁₂ branched alkenyl; C₉ to C₁₂ cycloalkyl-alkyl in which the cycloalkyl is C₃ to C₈ cycloalkyl positioned at either end or within the alkyl chain; or C₈ to C₁₄ aryl-alkyl in which the aryl is phenyl or naphthalenyl and is positioned at the either end or within the alkyl chain, and
wherein when p=4, each R is independently C₆ to C₁₀ straight-chain alkyl; C₆ to C₁₀ straight-chain alkenyl; C₆ to C₁₀ branched alkyl; C₆ to C₁₀ branched alkenyl; C₉ to C₁₀ cycloalkyl-alkyl in which the cycloalkyl is C₃ to C₈ cycloalkyl positioned at either end or within the alkyl; or C₈ to C₁₂ aryl-alky in which the aryl is phenyl or naphthalenyl and is positioned at the either end or within the alkyl chain.

Some embodiments specifically include one or more species or subgenera based on specific choices of R, X, Y, m, n, o, p, and/or carbon chain length, structure, or saturation. Other embodiments specifically exclude one or more species or subgenera based on specific choices of R, X, Y, m, n, o, p, and/or carbon chain length, structure, or saturation. In some embodiments, when p is 1, each R is independently C₈ to C₁₂, C₁₃, or C₁₄ straight-chain alkyl. In some embodiments, each R from a nearest common branch point is the same. In some embodiments, each R is the same.

In some embodiments, the ionizable cationic lipid has a structure of Formula 1a

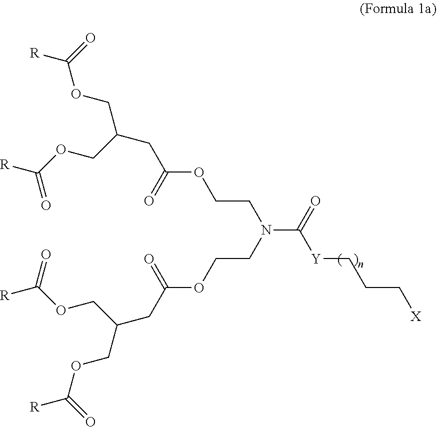

wherein each R is independently C₆ to C₁₆ straight-chain alkyl; C₆ to C₁₆ straight-chain alkenyl; C₆ to C₁₆ branched alkyl; C₆ to C₁₆ branched alkenyl; C₉ to C₁₆ cycloalkyl-alkyl in which the cycloalkyl is C₃ to C₈ cycloalkyl positioned at either end or within the alkyl chain; or C₈ to C₁₈ aryl-alkyl in which the aryl is phenyl or naphthalenyl and is positioned at either end or within the alkyl chain,
Y is O, NH, N—CH₃, or CH₂,
n is an integer from 0 to 4,
X is

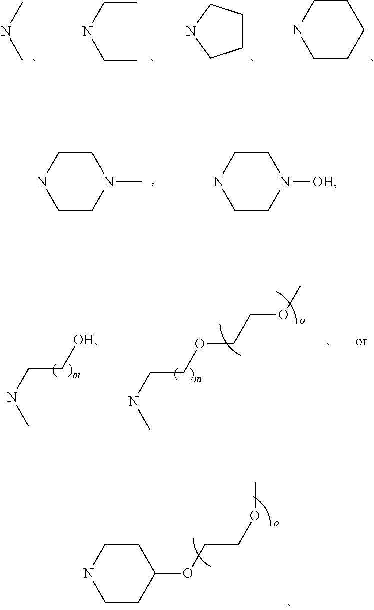

m is an integer from 1 to 3, and
is an integer from 1 to 4.

In other aspects, an ionizable cationic lipid has a structure of Formula 2,

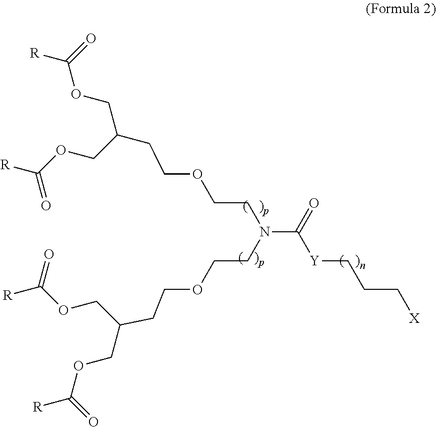

wherein Y is O, NH, N—CH₃, or CH₂,
n is an integer from 0_to 4,
X is

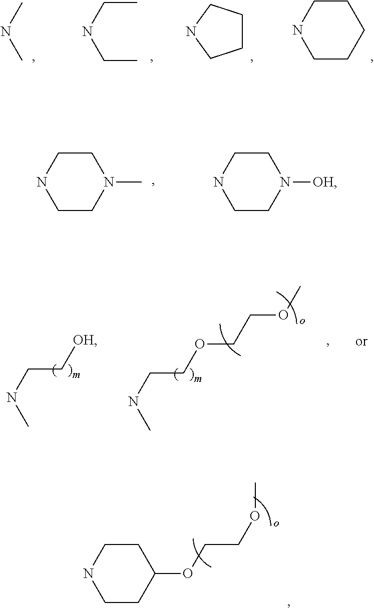

m is an integer from 1 to 3,
is an integer from 1 to 4,
p is an integer from 1 to 4,
wherein when p=1, each R is independently C₆ to C₁₆ straight-chain alkyl; C₆ to C₁₆ straight-chain alkenyl; C₆ to C₁₆ branched alkyl; C₆ to C₁₆ branched alkenyl; C₉ to C₁₆ cycloalkyl-alkyl in which the cycloalkyl is C₃ to C₈ cycloalkyl positioned at either end or within the alkyl chain; or C₈ to C₁₈ aryl-alkyl in which the aryl is phenyl or naphthalenyl and is positioned at either end or within the alkyl chain,
wherein when p=2, each R is independently C₆ to C₁₄ straight-chain alkyl; C₆ to C₁₄ straight-chain alkenyl; C₆ to C₁₄ branched alkyl; C₆ to C₁₄ branched alkenyl; C₉ to C₁₄ cycloalkyl-alkyl in which the cycloalkyl is C₃ to C₈ cycloalkyl positioned at the either end or within the alkyl chain; or C₈ to C₁₆ aryl-alkyl in which the aryl is phenyl or naphthalenyl and is positioned at either end or within the alkyl chain,
wherein when p=3, each R is independently C₆ to C₁₂ straight-chain alkyl; C₆ to C₁₂ straight-chain alkenyl; C₆ to C₁₂ branched alkyl; branched C₆ to C₁₂ alkenyl; C₉ to C₁₂ cycloalkyl-alkyl in which the cycloalkyl is C₃ to C₈ cycloalkyl positioned at either end or within the alkyl chain; or C₈ to C₁₄ aryl-alkyl in which the aryl is phenyl or naphthalenyl and is positioned at the either end or within the alkyl chain, and
wherein when p=4, each R is independently C₆ to C₁₀ straight-chain alkyl; straight-chain C₆ to C₁₀ alkenyl; C₆ to C₁₀ branched alkyl; C₆ to C₁₀ branched alkenyl; C₉ to C₁₀ cycloalkyl-alkyl in which the cycloalkyl is C₃ to C₈ cycloalkyl positioned at either end or within the alkyl; or C₈ to C₁₂ aryl-alky in which the aryl is phenyl or naphthalenyl and is positioned at the either end or within the alkyl chain.

Some embodiments include one or more species or subgenera based on specific choices of R, X, Y, m, n, o, p, and/or carbon chain length, structure, or saturation. Other embodiments specifically exclude one or more species or subgenera based on specific choices of R, X, Y, m, n, o, p, and/or carbon chain length, structure, or saturation. In some embodiments, each R from a nearest common branch point is the same. In some embodiments, each R is the same.

In some embodiments, the ionizable cationic lipid has a structure of Formula 2a

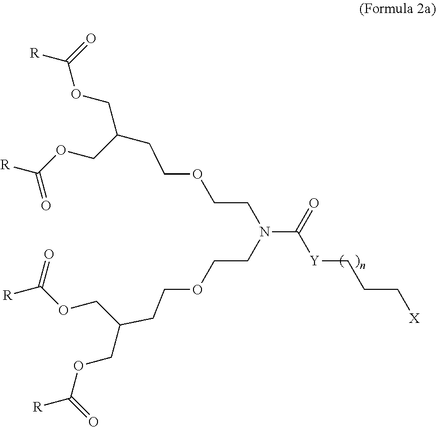

wherein R is C₆ to C₁₆ straight-chain alkyl; C₆ to C₁₆ straight-chain alkenyl; C₆ to C₁₆ branched alkyl; branched C₆ to C₁₆ alkenyl; C₉ to C₁₆ cycloalkyl-alkyl in which the cycloalkyl is C₃ to C₈ cycloalkyl positioned at either end or within the alkyl chain; or C₈ to C₁₈ aryl-alkyl in which the aryl is phenyl or naphthalenyl and is positioned at either end or within the alkyl chain,
Y is O, NH, N—CH₃, or CH₂,
n is an integer from 0 to 4,
X is

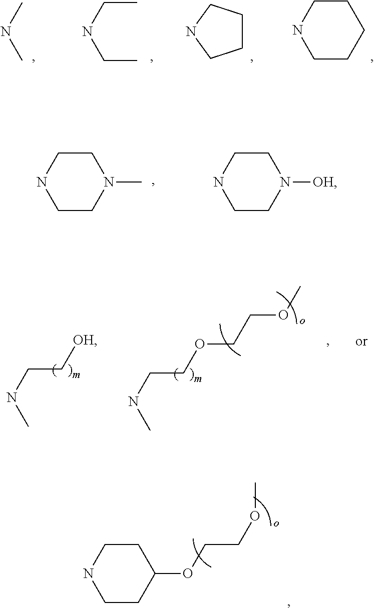

m is an integer from 1 to 3, and
is an integer from 1 to 4.

In further aspects, an ionizable cationic lipid has a structure of Formula 3,

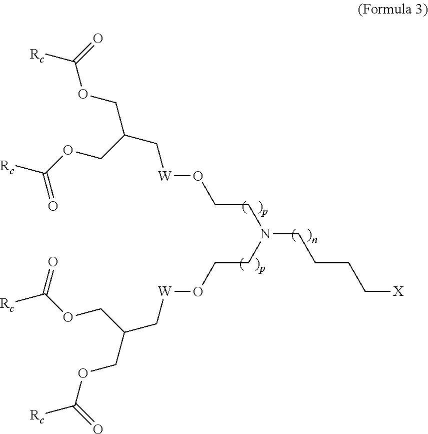

wherein W is C═O or CH₂,
n is an integer from 0 to 4,
X is

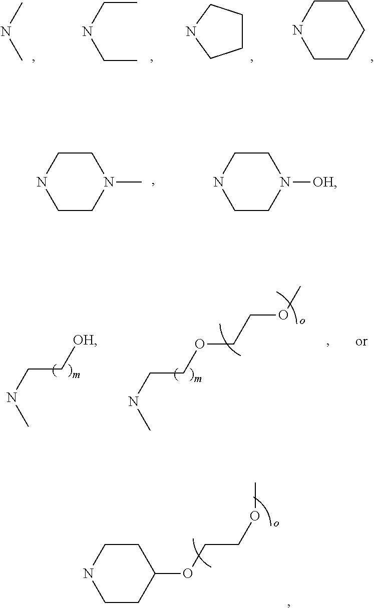

m is an integer from 1 to 3,
is an integer from 1 to 4,
p is an integer from 1 to 4,
wherein when p=1, each R_{c} is independently C₈ to C₁₈ straight-chain alkyl; C₈ to C₁₈ straight-chain alkenyl; C₈ to C₁₈ branched alkyl; C₈ to C₁₈ branched alkenyl; C₁₁ to C₁₈ cycloalkyl-alkyl in which the cycloalkyl is C₃ to C₈ cycloalkyl positioned at either end or within the alkyl chain; or C₁₀ to C₂₀ aryl-alkyl in which the aryl is phenyl or naphthalenyl and is positioned at either end or within the alkyl chain,
wherein when p=2, each R_{c} is independently C₈ to C₁₆ straight-chain alkyl; C₈ to C₁₆ straight-chain alkenyl; C₈ to C₁₆ branched alkyl; C₈ to C₁₆ branched alkenyl; C₁₁ to C₁₆ cycloalkyl-alkyl in which the cycloalkyl is C₃ to C₈ cycloalkyl positioned at the either end or within the alkyl chain; or C₁ to C₁₈ aryl-alkyl in which the aryl is phenyl or naphthalenyl and is positioned at either end or within the alkyl chain,
wherein when p=3, each R_{c} is independently C₈ to C₁₄ straight-chain alkyl; C₈ to C₁₄ straight-chain alkenyl; C₈ to C₁₄ branched alkyl; C₈ to C₁₄ branched alkenyl; C₁₁ to C₁₄ cycloalkyl-alkyl in which the cycloalkyl is C₃ to C₈ cycloalkyl positioned at either end or within the alkyl chain; or C₁₀ to C₁₆ aryl-alkyl in which the aryl is phenyl or naphthalenyl and is positioned at the either end or within the alkyl chain, and
wherein when p=4, each R_{c} is independently C₈ to C₁₂ straight-chain alkyl; C₈ to C₁₂ straight-chain alkenyl; C₈ to C₁₂ branched alkyl; C₈ to C₁₂ branched alkenyl; C₁₁ to C₁₂ cycloalkyl-alkyl in which the cycloalkyl is C₃ to C₈ cycloalkyl positioned at either end or within the alkyl; or C₁₀ to C₁₄ aryl-alky in which the aryl is phenyl or naphthalenyl and is positioned at the either end or within the alkyl chain.

Some embodiments include one or more species or subgenera based on specific choices of R_{c}, W, X, m, n, o, p, and/or carbon chain length, structure, or saturation. Other embodiments specifically exclude one or more species or subgenera based on specific choices of R_{c}, W, X, m, n, o, p, and/or carbon chain length, structure, or saturation. In some embodiments, each R_{c} from a nearest common branch point is the same. In some embodiments, each R_{c} is the same.

In some embodiments, the ionizable cationic lipid has a structure of Formula 3a

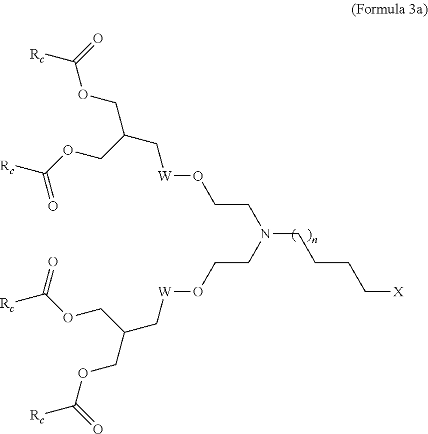

wherein R_{c} is C₈ to C₁₈ straight-chain alkyl; C₈ to C₁₈ straight-chain alkenyl; C₈ to C₁₈ branched alkyl; C₈ to C₁₈ branched alkenyl; C₁₁ to C₁₈ cycloalkyl-alkyl in which the cycloalkyl is C₃ to C₈ cycloalkyl positioned at either end or within the alkyl chain; or C₁₀ to C₂₀ aryl-alkyl in which the aryl is phenyl or naphthalenyl and is positioned at either end or within the alkyl chain,
W is C═O, or CH₂,
n is an integer from 0 to 4,
X is

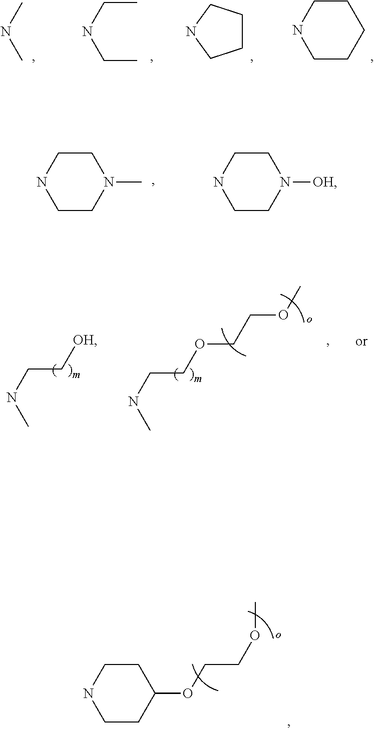

m is an integer from 1 to 3, and
is an integer from 1 to 4.

With respect to each of the forgoing aspects, in some embodiments, all four R groups are identical. In other embodiments, the two R_{c} groups stemming from a first branchpoint are identical to each other and the two R_{c} groups from a second branchpoint are identical to each other, but the R_{c} groups stemming from the first branchpoint are different than the R groups stemming from the second branchpoint.

With respect to each of the forgoing aspects, some embodiments are limited to one, or a subset, of the alternatives for R_{c}, W, X, Y, m, n, o, and/or p, as applicable. Other embodiments specifically exclude one, or a subset, of the alternatives for R_{c}, W, X, Y, m, n, o, p, and/or carbon chain length, structure, or saturation, as applicable. Each range of carbon chain length is meant to convey embodiments of all individual lengths and subranges therein.

With respect to each of the foregoing aspects and embodiments, in some instances R_{c} is straight-chain alkyl and in further instances the chain is unsubstituted. In still further instances, R_{c} is C₈ or C₉ or C₁₀ to C₁₂.

With respect to each of the foregoing aspects and embodiments, in some instances X is

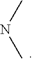

With respect to each of the foregoing aspects and embodiments, in some instances Y is O and in other instances Y is NH or N—CH₃.

c Log D is a calculated measure of lipophilicity that takes into account the state of ionization of the molecule at a particular pH, predicting partitioning of the lipid between water and octanol as a function of pH. More specifically, c Log D is calculated at a specified pH based on c Log P and c-pKa. (Log P is the partition coefficient of a molecule between aqueous and lipophilic phases usually considered as octanol and water.) When higher basicity of the ionizable lipid is desired, it should be balanced by greater lipophilicity as represented by a higher c Log D value. Balance of basicity and lipophilicity is used herein to maximize LNP function, including both stability of the LNP and release of the cargo (e.g., a nucleic acid) upon uptake by a cell. Accordingly, as m, n, or p increases, overall lipophilicity of ionizable cationic lipids disclosed herein, as represented by c Log D, can be balanced by shorter chain lengths for R. Some embodiments of the ionizable cationic lipid species encompassed by Formulas 1-3 have a c Log D ranging from about of 9 to about 18 or about 9 to about 22 calculated using ACD Labs Structure Designer v 12.0, c Log P was calculated using ACD Labs Version B; c Log D was calculated at pH 7.4.

A measured pKa of 6 to 7 for an LNP carrying a nucleic acid load ensures that the ionizable cationic lipid in the LNP will remain essentially neutral in the blood stream and interstitial spaces but ionize after uptake into cells as the endosomes acidify. Upon acidification in the endosomal space, the lipid becomes protonated, and associates more strongly with the phosphate backbone of the nucleic acid, which destabilizes the structure of the LNP and promotes nucleic acid release from the LNP into the cell cytoplasm (also referred to as endosomal escape). Thus, the herein disclosed ionizable cationic lipids constitute means for destabilizing LNP structure (when ionized) or means for promoting nucleic acid release or endosomal escape.

Ionizable cationic lipids of this disclosure have a branched structure to give the lipid a conical rather than cylindrical shape and such structure helps promote endosomolytic activity. The greater the endosomolytic activity, the more efficient release of the nucleotide cargo.

To promote biodegradability and minimize the accumulation of ionizable cationic lipids of this disclosure, the fatty acid tails are designed to comprise esters in a position that minimizes steric hinderance of ester cleavage. For example, while a single fatty acid tail will tend to extend away from the ester carbonyl, the presence of two tails leads to the tails extending in opposite directions as this is an energetically favorable conformation. This means one of the tails may extend toward the carbonyl and sterically hinder cleavage of the ester. Accordingly, large branches immediately adjacent to the ester carbonyl were avoided. In positioning the ester(s) within the lipid, consideration was also given to potential degradation products to avoid the generation of toxic compounds, such as formaldehyde.

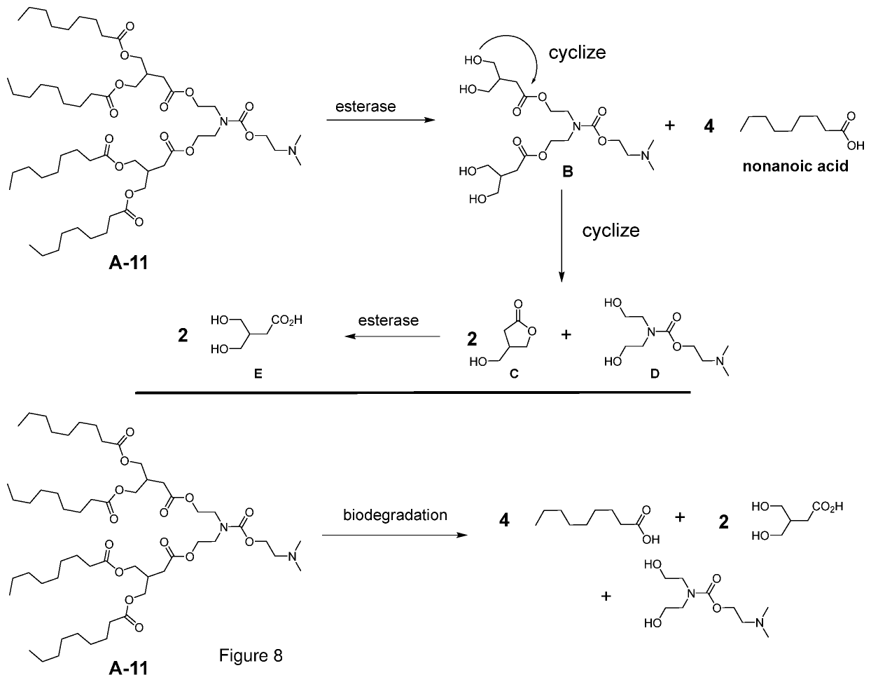

Another consideration potentially contributing to tolerability of the lipid is the extent to which ester cleavage or other catabolism generates fragments or byproducts and whether such fragments or byproducts can be eliminated from the body without involving oxidative degradation in the liver. The ionizable cationic lipids of this disclosure are expected to be readily biodegradable- and the fragments easily cleared. For example, FIG. 8 depicts that esterase cleavage or other hydrolysis of compound A-11 would be predicted to produce tetra-alcohol B and 4 equivalents of nonanoic acid. Cyclization should then result in the production of 2 equivalents of butyrolactone C and 1 equivalent of diol D. Esterase hydrolysis of C would result in the production of 2 equivalents of diol-acid E. The products of the biodegradation of A-11 are shown collectively below the line in FIG. 8. All of these entities are small and polar and expected-to be cleared from the body without the need for hepatic oxidation or conjugation. These considerations gain importance if the LNP of tLNP will be used in a chronic dosing regimen.

An advantage of relying, at least in part, on ionizable cationic lipids of this disclosure is that it avoids the toxicity associated with quaternary ammonium cationic lipids. Some LNPs based on such lipids, which are effectively permanently cationic, have displayed a fatal hyperacute toxicity in laboratory animals. By use of ionizable cationic lipids of this disclosure in LNP, use of quaternary ammonium cationic lipids can be substantially reduced mitigating or avoiding toxicity. In certain embodiments, use of a LNP or tLNP of this disclosure causes no detectable toxicity to cells or in a subject. In certain embodiments, use of a LNP or tLNP of this disclosure causes no more than mild toxicity to cells or in a subject that is asymptomatic or induces only mild symptoms that do not require intervention. In certain embodiments, use of a LNP or tLNP of this disclosure causes no more than moderate toxicity to cells or in a subject which may impair activities of daily living that requires only minimal, local, or non-invasive interventions.

The relationship between the efficacy and toxicity of a drug is generally expressed in terms of therapeutic window and therapeutic index. Therapeutic window is the dose range from the lowest dose that exhibits a detectable therapeutic effect up to the maximum tolerated dose (MTD); the highest dose that will the desired therapeutic effect without producing unacceptable toxicity. Most typically therapeutic index is calculated as the ratio of LD50:ED50 when based on animal studies and TD50:ED50 when based on studies in humans (though this calculation could also be derived from animal studies and is sometime called the protective index), where LD50, TD50, and ED50 are the doses that are lethal, toxic, and effective in 50% of the tested population, respectively. These concepts are applicable whether the toxicity is based on the active agent itself or some other component of the drug product, as for example, the LNP or its components. For any inherent level of toxicity of the disclosed lipids or LNPs themselves, an increase in the efficiency of delivering the nucleic acid into the cytoplasm will improve the therapeutic window or index, as an effective amount of the nucleic acid would be deliverable with a smaller dosage of LNP (and its component lipids).

Toxicities and adverse events are sometimes graded according to a 5-point scale. A grade 1 or mild toxicity is asymptomatic or induces only mild symptoms; may be characterized by clinical or diagnostic observations only; and intervention is not indicated. A grade 2 or moderate toxicity may impair activities of daily living (such as preparing meals, shopping, managing money, using the telephone, etc.) but only minimal, local, or non-invasive interventions are indicated. Grade 3 toxicities are medically significant but not immediately life-threatening; hospitalization or prolongation of hospitalization is indicated; activities of daily living related to self-care (such as bathing, dressing and undressing, feeding oneself, using the toilet, taking medications, and not being bedridden) may be impaired. Grade 4 toxicities are life-threatening and urgent intervention is indicated. Grade 5 toxicity produces an adverse event-related death. Thus, in various embodiments, by use of the disclosed LNP and tLNP a toxicity is confined to grade 2 or less, grade 1 or less, or produces no observation of the toxicity.

In some instances, a LNP and tLNP of this disclosure is used according to a specified regimen, provided at a particular dosage, or administered via a particular route of administration.

### Methods of Making Ionizable Cationic Lipids

Structural symmetries and convergent rather than linear synthesis pathways can be used to simplify the synthesis of the ionizable lipids.

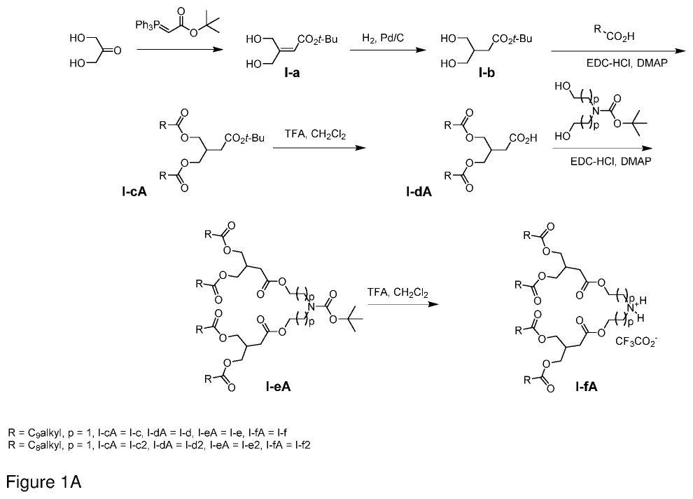

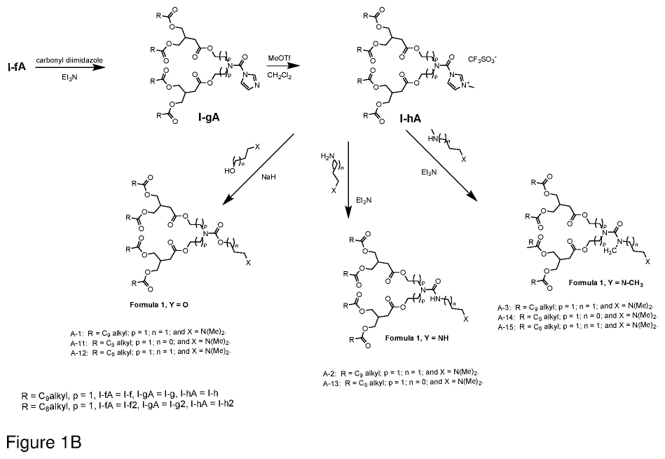

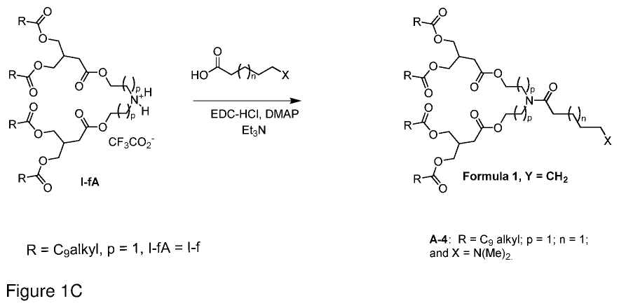

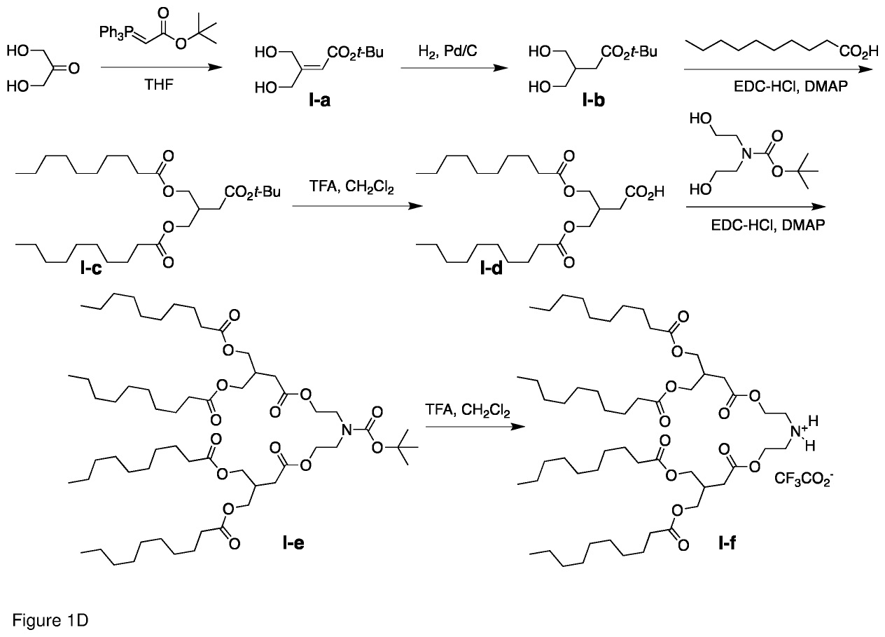

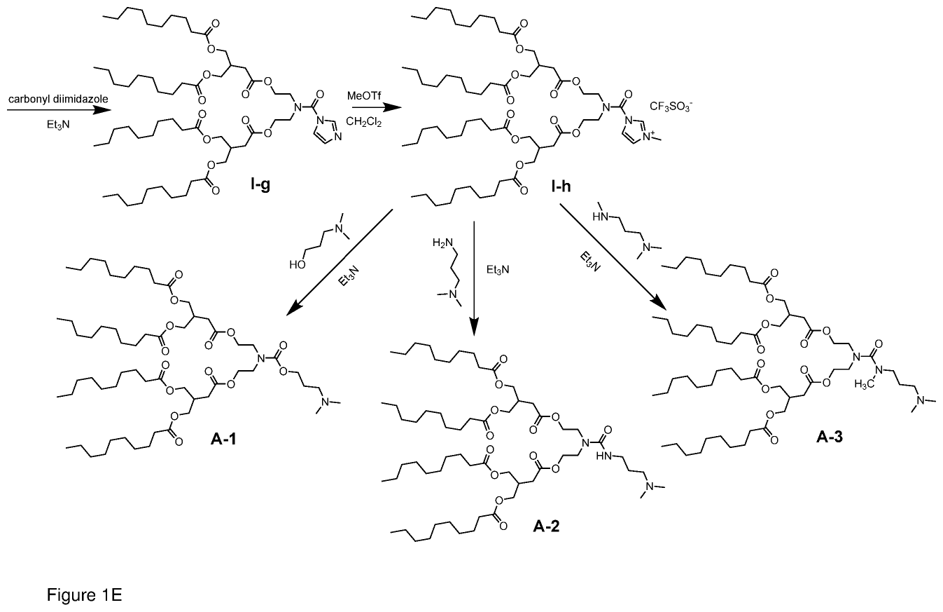

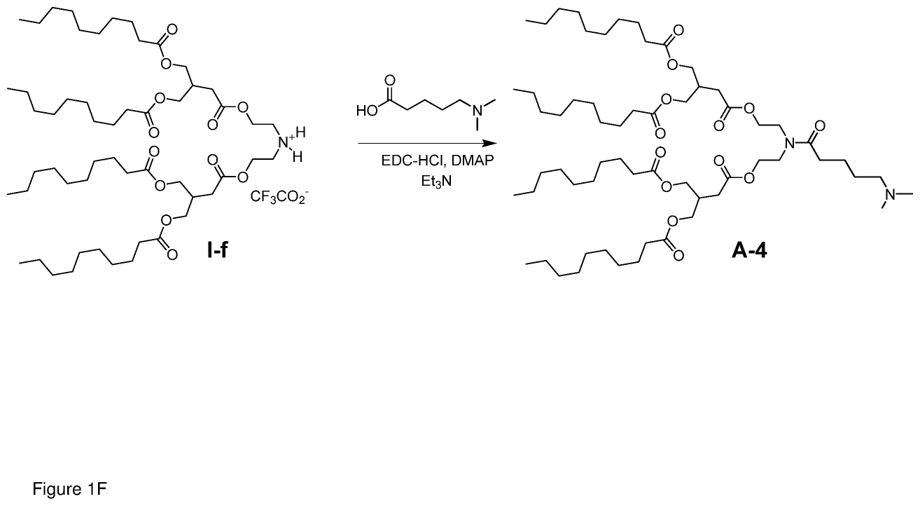

In certain aspects, the instant disclosure provides a method of synthesizing an ionizable cationic lipid of Formula 1. In some embodiments, the method comprises converting an intermediate having a structure of I-fA to the ionizable cationic lipid of Formula 1. In some embodiments, the method further comprises synthesizing the intermediate having a structure of I-fA (e.g., FIG. 1A).

In certain embodiments of the synthesis method of the ionizable cationic lipid of Formula 1, Y═O, NH, or N—CH₃, and the method further comprises reacting I-fA with carbonyl diimazole to provide I-gA. In certain embodiments, the method further comprises coupling I-gA and X—(CH₂)ₙ₊₂—YH. In certain embodiments, the coupling reaction of I-gA and X—(CH₂)ₙ₊₂—YH is performed in the presence of an alkylating agent. In certain embodiments, the alkylating agent is MeOTf, as shown in FIG. 1B. In certain embodiments, the coupling reaction comprises coupling an intermediate having a structure of I-hA with X—(CH₂)ₙ₊₂—YH to provide the ionizable cationic lipid of Formula 1, wherein Y═O, NH, or N—CH₃. In certain embodiments, the coupling reaction of I-hA with X—(CH₂)ₙ₊₂—YH is carried out in the presence of a base, e.g., without limitation, NaH, or Et₃N.

In certain embodiments of the synthesis method of the ionizable cationic lipid of Formula 1, Y═CH₂, the method comprises coupling an intermediate having a structure of I-fA with X—(CH₂)ₙ₊₃—COOH to provide the ionizable cationic lipid of Formula 1. In certain embodiments, the coupling method is carried out in the presence of DMAP and Et₃N, e.g., as shown in FIG. 1C.

In certain embodiments of the synthesis method of I-fA, the method comprises coupling an intermediate of I-dA with (HO—CH₂—(CH₂)ₚ)₂—N-PG to provide an amine-protected derivative of I-fA, wherein PG is a protecting group of amine. In certain embodiments, PG is —CO₂t-Bu as shown in FIG. 1A. In certain embodiments of the synthesis method of I-fA, the amine-protected derivative of I-fA is deprotected to provide I-fA. For example, the deprotecting reagent can be TFA in dimethyl chloride as shown in FIG. 1A. In certain embodiments, the method further comprises synthesis of I-dA.

In certain embodiments of the synthesis method of I-dA comprises preparing a carboxylic acid derivative of I-dA wherein the carboxylic acid moiety of I-dA is protected with a protecting group that can be deprotected selectively over the hydrolysis of the R—COO— moiety. In certain embodiments, the carboxylic acid derivative of I-dA is I-cA which is a t-Butyl ester of I-dA, e.g., see FIG. 1A. In certain embodiments, the carboxylic acid derivative of I-dA is prepared by reacting the desired diol carboxylic acid derivative (e.g., I-b, wherein the carboxylic acid derivative is a t-Butyl ester, in other embodiments, the derivative can be other forms) and R—COOH. In certain embodiments, the diol carboxylic acid derivative is prepared by hydrogenation of an alkenyldiol carboxylic acid derivative (e.g., I-a, wherein the carboxylic acid derivative is a t-Butyl ester, in other embodiments, the derivative can be other forms). In certain embodiments, the alkenyldiol carboxylic acid derivative is prepared by reacting dihydroxyacetone and an alkyloxycarbonyl methylene triphenyl phosphorane (e.g., the alkyl can be t-butyl as shown in FIG. 1A).

In some embodiments, the method of synthesizing an ionizable cationic lipid of Formula 1 proceeds according to the synthetic scheme of FIGS. 1D-F. In some embodiments, the method of synthesizing an ionizable cationic lipid of Formula 1 proceeds according to Examples 5-16 and 24 (for example, Compounds A-11 thru A-15), 17-23 (for example, Compound A-2), or 25-33 (for example, Compound A-16); analogs of these Compounds with different m, n, o, p, R, X, and/or Y can be made by substituting reactants as described herein. In some embodiments, the method is a method of synthesizing an ionizable cationic lipid of Formula 1a. In some instances, the method is a method of synthesizing Compound A-1, Compound A-2, Compound A-3, Compound A-4, Compound A-11, Compound A-12, Compound A-13, Compound A-14, Compound A-15 or Compound A-16. In some embodiments, the method specifies only a single step, or subset of steps, depicted in FIGS. 1D-F or Examples 5-16 and 24, 17-23, or 25-33, resulting in the final product.

Further aspects are intermediates I-cA, I-dA, I-eA, I-fA, I-gA, I-hA, I-d, I-d2, I-e, I-e2, I-f, I-f2, I-g, I-g2, I-h, and I-h2 of the synthetic scheme of FIGS. 1A-F, Examples 8-12, and Examples 18-22, wherein the substitution groups are defined the same as Formula 1 unless specified otherwise.

A further aspect is a method of synthesizing an intermediate of the synthetic scheme of FIGS. 1D-F, wherein the intermediate is I-d, I-e, I-f, 1-g, or I-h. In some embodiments, the method specifies only a final step to generate the intermediate as depicted in FIGS. 1D-F. In other embodiments, the method specifies all or a subset of the steps as depicted in FIGS. 1D-F to reach the intermediate. Further embodiments relate to analogues of the intermediates I-d, I-e, I-f, 1-g, or 1-h appropriate to final products with differing X, n, or p, such as I-eA or I-gA, for example I-d2, I-e2, I-f2, I-g2, or I-h2 as shown in Examples 8-12 and Examples 18-22.

In the synthetic scheme depicted in FIGS. 1D-F, the value of p is 1, resulting from the coupling of intermediate I-d with BOC-blocked di-ethanolamine. Compounds in which p is 2 to 4 can be synthesized by substituting the appropriately sized BOC-blocked dialkylamino alcohol; that is, 3,3′-azanediylbis(propan-1-ol), 4,4′-azanediylbis(butan-1-ol), and 5,5′-azanediylbis(pentan-1-ol), respectively.

In the synthetic scheme depicted in FIGS. 1D-F, the value of n is 1 resulting from the reaction of intermediate I-h with 3-dimethylamino-1-propanol, N,N-dimethyl-1,3-propanediamine, N,N,N′-trimethyl-1,3-propanediamine, in the presence of base to generate Compounds A-1 to A-3, respectively, in which Y is O, NH, or N—CH₃, respectively. Compounds in which n is 0 or 2 to 4 can be synthesized by substituting the propanediamine moiety with an analogous C₂, C₄, C₅ or C₆ moiety. Compound A-4, in which Y is CH₂, is obtained by reacting a salt of intermediate I-f with 5-dimethylamino-pentanoic acid. Compounds in which n is 0 or 2 to 4 can be synthesized by substituting the pentanoic acid moiety with an analogous C₄, C₆, C₇, or C₈ moiety.

In the synthetic scheme depicted in FIGS. 1D-F, R is C₉, resulting from the use of decanoic acid in the conversion of intermediate I-b to intermediate I-c. Substitution of -oic acids of the corresponding chain length and structure can be used to obtain R of C₆-C₈ or C₁₀-C₁₈, as appropriate.

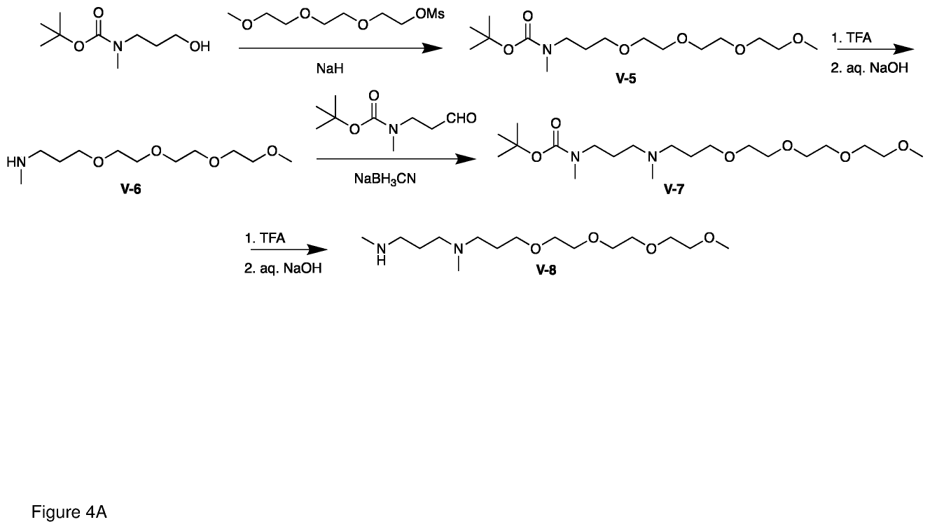

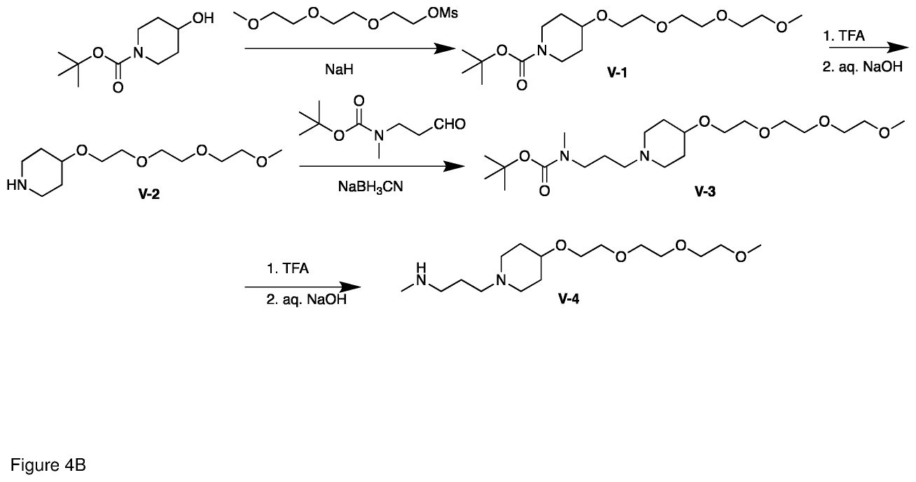

In the synthetic scheme depicted in FIGS. 1D-F, X is N(CH₃)₂. Compounds according to Formula 1 having alternative definitions of X can be synthesized by reacting alternative head group pieces from Tables 1-3 with I-h to obtain analogues of Compounds A-1 to A-3, respectively, or reacting alternative head group pieces from Table 4 with I-f to obtain analogues of Compound A-4, as disclosed in Example 1 (below). Synthesis of head group pieces not previously disclosed in the art can be made analogously to their shorter congeners or, for polyethylene glycol-containing head group pieces, made according to the synthetic schemes shown in FIGS. 4A-B and disclosed in Example 4, or as described in Examples 25-32, (below).

Synthesis of the polyethylene glycol-containing head group pieces require polyethylene glycol amines and related reagents that have not been previously described. Thus, some aspects are intermediates V-5, V-5a, V-6, V-6A, V-7, V-7a, V-8, V-8a, V-12, V-13, V-14, or V-15 and methods of their synthesis according to the synthetic schemes shown in FIGS. 4A-B and disclosed in Example 4, or as described in Examples 25-32.

To synthesize intermediate I-e and its analogs having the structure of I-eA:

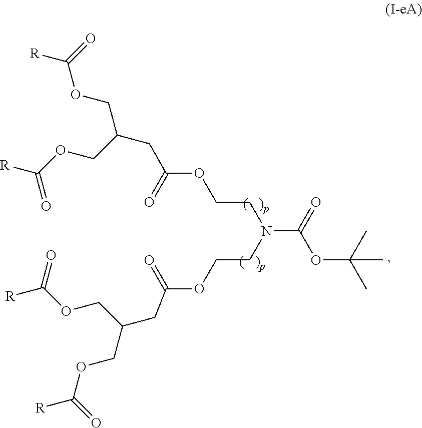

wherein p is an integer from 1-4 and R is defined the same as for Formula 1, dihydroxyacetone is reacted with tert-butoxycarbonylmethylene triphenylphosphorane of to give alkene I-a. Hydrogenation, for example in the presence of Pd/C in ethyl acetate, leads to I-b (FIG. 1D). Coupling of I-b with the appropriate carboxylic acid for the desired R in the presence of EDC-HCl and DMAP in dichloromethane leads to tri-ester I-c (FIG. 1D) or its analogue with different R as shown in I-cA. Hydrolysis of the t-butyl ester with TFA in dichloromethane results in a critical mono-acid, I-d (FIG. 1D) or its analogue with different R. A coupling of I-d or its analog with commercially available BOC-blocked di-ethanolamine (FIG. 1D) or the appropriately sized BOC-blocked dialkylamino alcohol for n=2 to 4, in dichloromethane, affords compounds with a structure of I-eA.

To synthesize intermediate 1-g and its analogs with different R and/or p, that is an intermediate with the structure:

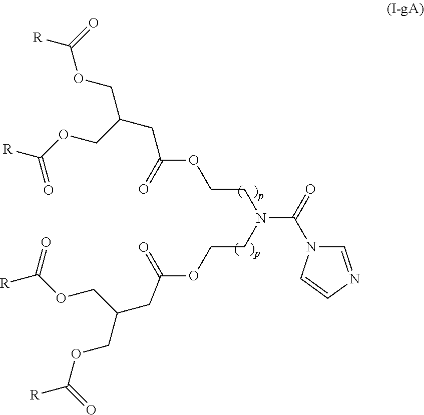

wherein p is an integer from 1-4 and R is defined as for Formula 1, an intermediate with a structure of I-eA is treated with TFA in dichloromethane to remove the BOC protecting group, giving the salt I-f (FIG. 1D) or an analog thereof with different R and/or p (e.g., intermediate with a structure of I-fA as shown in FIG. 1A). That product is then converted into acyl-imidazolide 1-g (FIG. 1E) or an acyl-imidazolide with a structure of I-gA (FIG. 1B) upon reaction with carbonyl diimidazole and triethylamine in dichloromethane.

To complete the syntheses of Compounds A-1 to A-3 and their analogues with different R, and/or p, the needed reactive intermediate is obtained by the reaction of an intermediate with the structure of I-gA with methyl triflate to produce acyl-imidazolium I-h (FIG. 1E) or an analogue thereof with different R such as I-h2 for R of straight-chain C₈ (Example 12) or I-hA (FIG. 1B). For p=1 and n=1, the acyl-imidazolium intermediate is then reacted with: 3-dimethylamino-1-propanol in the presence of triethylamine, to provide Compound A-1 (FIG. 1E) or analogues with different p (e.g., FIG. 1B); with N,N-dimethyl-1,3-propanediamine and triethylamine to provide Compound A-2 (FIG. 1E; see also Example 23) or analogues with different p (e.g., FIG. 1B); or with N,N,N′-trimethyl-1,3-propanediamine and triethylamine to provide Compound A-3 (FIG. 1E), in each case in dichloromethane, or analogues with different p (FIG. 1B).

To complete the synthesis of Compound A-4 (FIG. 1F) or their analogues with different R and/or p, the salt I-f (FIG. 1C), or its analogs with different R and/or p, is reacted with 5-dimethylamino-pentanoic acid in the presence of EDC-HCl, DMAP, and triethylamine, in dichloromethane, to provide Compound A-4 (FIG. 1F) or analogues thereof with different R and/or p (FIG. 1C).

The reagent substitutions used to obtain analogues of Compounds A-1 to A-4 with different X, Y, and/or n are described above and are applicable to the foregoing syntheses for obtaining analogues of Compounds A-1 to A-4 with different R, and/or p (FIGS. 1A-C).

Such analogues include Compounds A-11 to A-15 in which R is straight-chain C₈ rather than straight-chain C₉ as in A-1 to A-4. Additionally, A-11 differs from A-1 in that n=0 instead of n=1. A-12 differs from A-1 only in R. A-13 additionally differs from A-2 in that n=0 instead of n=1. A-14 additionally differs from A-3 in that n=0 instead of n=1. A-15 differs from A-3 only in R.

To complete the synthesis of Compound A-11 the acyl-imidazolium I-h2 is reacted with 2-dimethylamino-ethanol in the presence of tetramethylethylene diamine, as in Example 13. Analogues of Compound A-11 retaining p=1 but with other R are made by substituting an acyl-imidazolium generated from the appropriate species of I-gA.

To complete the synthesis of Compound A-12 the acyl-imidazolium I-h2 is reacted with 3-dimethylamino-propanol in the presence of tetramethylethylene diamine, as in Example 14. Analogues of Compound A-12 retaining p=1 but with other R are made by substituting an acyl-imidazolium generated from the appropriate species of I-gA.

To complete the synthesis of Compound A-13 the acyl-imidazolium I-h2 is reacted with 2-dimethylamino-ethanol in the presence of triethylamine, as in Example 15. Analogues of Compound A-13 retaining p=1 but with other R are made by substituting an acyl-imidazolium generated from the appropriate species of I-gA.

To complete the synthesis of Compound A-14 the acyl-imidazolium I-h2 is reacted with N,N,N′-trimethylethylenediamine in the presence of triethylamine, as in Example 16. Analogues of Compound A-14 retaining p=1 but with other R are made by substituting an acyl-imidazolium generated from the appropriate species of I-gA.

To complete the synthesis of Compound A-15 the acyl-imidazolium I-h2 is reacted with N,N,N′-trimethypropylenediamine in the presence of triethylamine, as in Example 24. Analogues of Compound A-15 retaining p=1 but with other R are made by substituting an acyl-imidazolium generated from the appropriate species of I-gA.

In Compound A-16, a species of Formula 1a in which Y is N—CH₃, X is

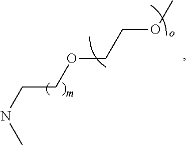

n is 1, m is 2, and o is 1, the head group piece terminates in a small polyethylene glycol moiety. Compound A-16 can be made according to the synthetic scheme presented in Example 4 and has also been synthesized as shown in Examples 25-33. In these latter examples, ultimately I-d2 is reacted with V-15 in the presence of DMAP and EDC-HCl in dichloromethane. Analogues of I-d2 with different hydrocarbon tails (e.g., 1-dA in FIG. 1A) can be used to generate analogues of Compound A-16 with different R.

To synthesize V-15 one can start from tert-butyl (3-hydroxypropyl)(methyl) carbamate by adding a cooled suspension of NaH in THF to it. Subsequently a solution of 2-methoxyethyl methanesulfonate in THF is slowly added and the mixture stirred at elevated temperature for an extended period of time. After cooling to room temperature, the reaction is quenched by careful addition of saturated aqueous NH₄Cl. The mixture is cast into ethyl acetate, the organic phase separated, the aqueous phase extracted with ethyl acetate and the combined organic phase washed with brine and dried over Na₂SO₄. Concentration of a filtrate produces crude V-5a which is dissolved in dichloromethane and dried onto silica gel. The silica gel is placed in a column and V-5a eluted with dichloromethane and concentrated to a yellow oil.

To synthesize V-6a, V-5a in dioxane is exposed slowly added acid, for example, HCl, stirred for several hours, and solvent removed. The crude V-6a is dissolved in dichloromethane and tert-butylmethyl(3-oxopropyl)carbamate is added. After a stirred incubation NaBH(OAc)₃ is added in several portions over a time interval and incubated further. Water is then added, and pH adjusted to 8 with Na₂CO₃. The mixture is extracted with dichloromethane, the organic phases combined and dried over Na₂SO₄, and solids removed by filtration. Silica gel is added to the filtrate and concentrated to dryness. The silica gel is then added to a column and V-7a eluted with a gradient of dichloromethane:methanol and dried to a yellow oil. V-7a is dissolved in dioxane and exposed to slowly added acid, for example, HCl. After incubation the solvent was removed to afford crude V-8a as a while solid.

To synthesize V-12, imidazole is added to a solution of diethanolamine in dichloromethane, stirred, and a solution of t-butyldimethylsilylchloride slowly added. The resulting solution was incubated and then the reaction quenched by addition of 10% aqueous NH₄OH. The organic phase was separated, and the aqueous phase extracted with dichloromethane. Combined organic phases are washed successively with saturated NH₄Cl and brine, and dried. Filtration and concentration affords V-12 as a clear, colorless oil.

CDI and Et₃N are added in order to a solution of V-12 in dichloromethane, the resulting solution incubated with stirring, and then cast into water. The organic phase was separated, and the aqueous phase extracted with dichloromethane. Combined organic phases are washed successively with saturated NH₄Cl and 5% aqueous NaHCO₃, and dried. Filtration and concentration affords V-13 as a pale yellow oil.

To a solution of cold V-13 in dichloromethane is slowly added MeOTf. After a stirred cold incubation a solution of Et₃N and V-8a in dichloromethane was slowly added. When the addition is complete the solution is warmed and incubated for several hours. Then the reaction mixture is cast into water and the organic layer removed. The aqueous layer is extracted with dichloromethane and combined organic phases concentrated. The resulting crude V-14 is dissolved in heptane and the solution extracted with MeOH/H₂O. Combined aqueous phases are then extracted with heptane and combined organic phases washed with brine and dried over MgSO₄. After filtration, silica gel is added to the filtrate and the mixture concentrated to dryness. The silica gel is then added to a column and V-14 eluted with a gradient of dichloromethane:methanol and the fractions containing V-14 concentrated to provide V-14 as a yellow oil.

To complete the synthesis of V-15, BF₃-OEt₂ is slowly added to a solution of V-14 in THF. The mixture is incubated with stirring for several hours and poured onto water. The pH is adjusted to 8.0 with saturated aqueous NaHCO₃ and the solvent removed to about a fifth of its original volume. The remaining solution is purified by flash chromatography using a water:acetonitrile gradient. Fractions containing V-14 are pooled and concentrated to provide V-14 as an off-white oil.

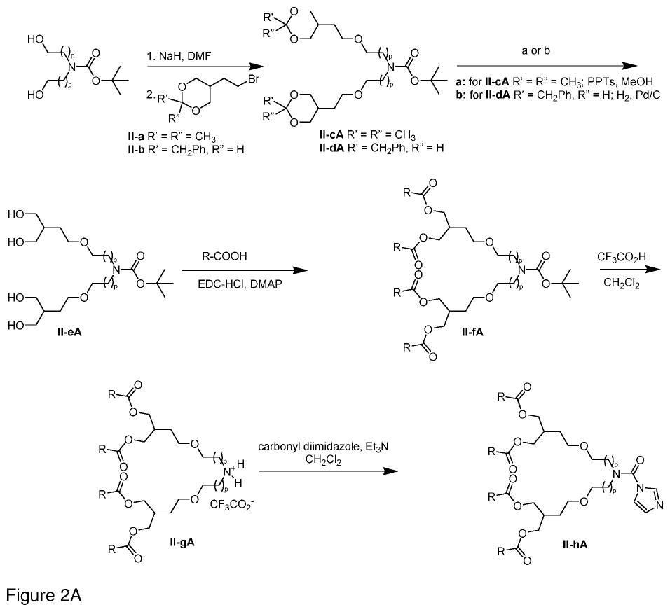

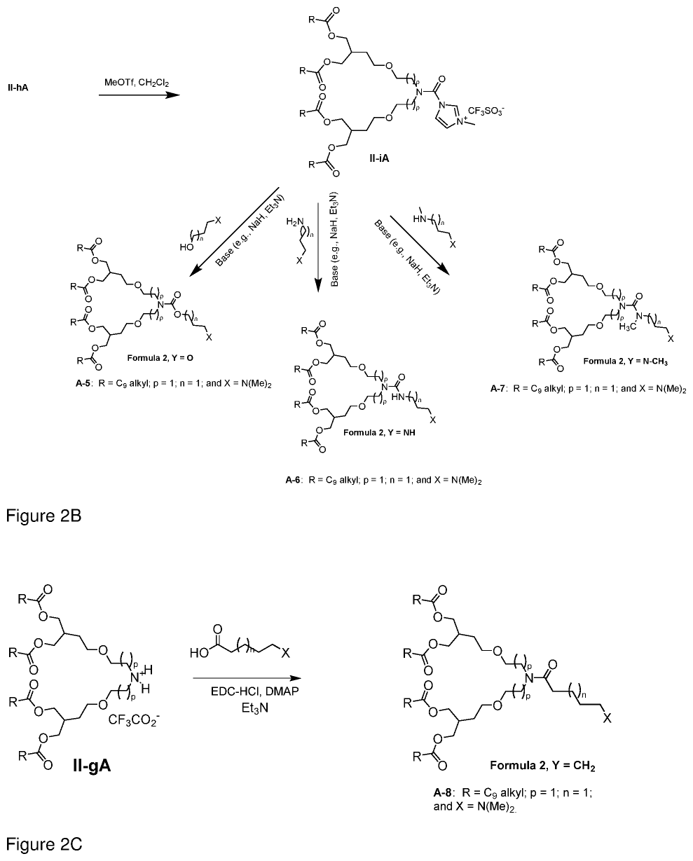

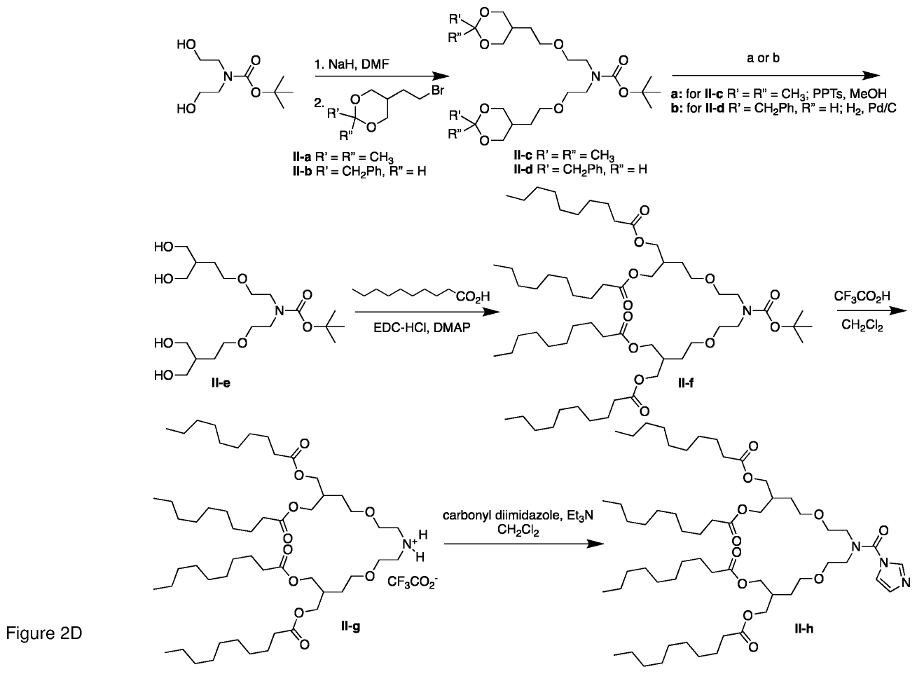

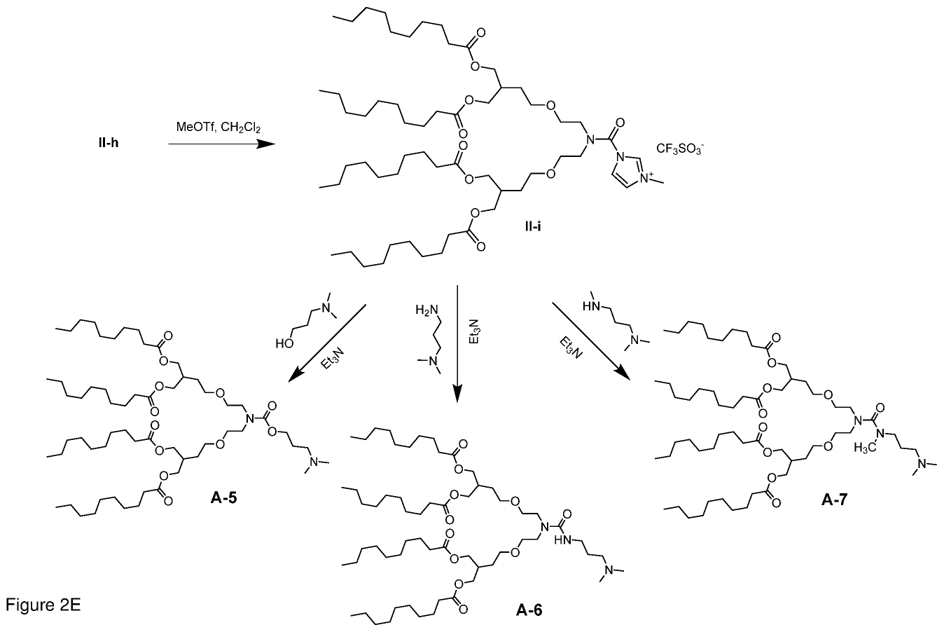

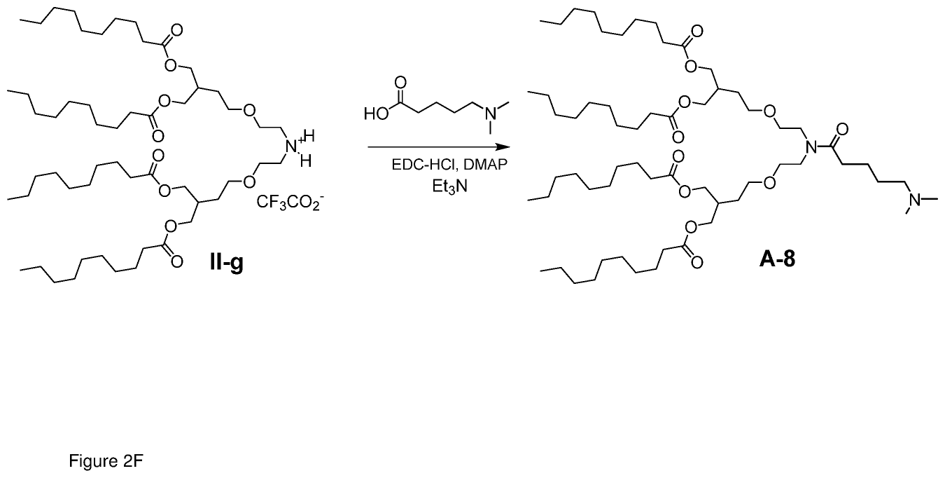

In certain aspects, the present disclosure provides a method of synthesizing an ionizable cationic lipid of Formula 2. In some embodiments, the method comprises converting an intermediate having a structure of II-gA to the ionizable cationic lipid of Formula 2. In some embodiments, the method further comprises synthesizing the intermediate having a structure of II-gA (FIG. 2A).

In certain embodiments of the synthesis method of the ionizable cationic lipid of Formula 2, Y═O, NH, or N—CH₃, and the method further comprises reacting II-gA with carbonyl diimazole to provide II-hA. In certain embodiments, the method further comprises coupling II-hA and X—(CH₂)ₙ₊₂—YH. In certain embodiments, the coupling reaction of II-hA and X—(CH₂)ₙ₊₂—YH is performed in the presence of an alkylating agent. In certain embodiments, the alkylating agent is MeOTf, as shown in FIG. 2B. In certain embodiments, the coupling reaction comprises coupling an intermediate having a structure of II-iA with X—(CH₂)ₙ₊₂—YH to provide the ionizable cationic lipid of Formula 2, wherein Y═O, NH, or N—CH₃. In certain embodiments, the coupling reaction of II-iA with X—(CH₂)ₙ₊₂—YH is carried out in the presence of a base, e.g., without limitation, NaH, or Et₃N. See FIG. 2B.

In certain embodiments of the synthesis method of the ionizable cationic lipid of Formula 2, Y═CH₂, the method comprises coupling an intermediate having a structure of II-gA with X—(CH₂)ₙ₊₃—COOH to provide the ionizable cationic lipid of Formula 2. In certain embodiments, the coupling method is carried out in the presence of DMAP and Et₃N, e.g., as shown in FIG. 2C.

In certain embodiments of the synthesis method of II-gA, the method comprises coupling an intermediate of II-eA with R—COOH to provide an amine-protected derivative of II-gA, also referred to as II-fA. In certain embodiments, the amine protecting group is —CO₂t-Bu as shown in FIG. 2A. In certain embodiments of the synthesis method of II-gA, II-fA, the amine-protected derivative of II-gA, is deprotected to provide II-gA. For example, the deprotecting reagent can be TFA in dimethyl chloride as shown in FIG. 2A. In certain embodiments, the method further comprises synthesis of II-eA.

In certain embodiments of the synthesis method of II-eA comprises preparing a derivative of II-eA wherein the hydroxyl groups of II-eA are protected (i.e., the OH-protected II-eA). For example, as shown in FIG. 2A, the OH-protected II-eA can be II-cA which can be prepared by reacting the sodium salt of BOC—N((CH₂)ₚ₊₁CH₂—OH)₂ with II-a. In another example, as shown in FIG. 2A, the OH-protected II-eA can be II-dA which can be prepared by reacting the sodium salt of BOC—N((CH₂)ₚ₊₁CH₂—OH)₂ with II-b.

In some embodiments, the method of synthesizing an ionizable cationic lipid of Formula 2 proceeds according to the synthetic scheme of FIGS. 2A-F. In some embodiments, the method is a method of synthesizing an ionizable cationic lipid of Formula 2a. In some instances, the method is a method of synthesizing Compound A-5, Compound A-6, Compound A-7, or Compound A-8. In some embodiments, the method specifies only a single step, or subset of steps, depicted in FIGS. 2A-F.

In further aspects, the present disclosure provides methods of synthesizing an intermediate of the synthetic scheme of FIGS. 2D-F, wherein the intermediate is II-e, II-f, II-g, II-h, or II-i. In some embodiments, the method comprises only a final step to generate the intermediate as depicted in FIGS. 2D-F. In other embodiments, the method comprises all or a subset of the steps as depicted in FIGS. 2D-F to reach the intermediate.

In the synthetic scheme depicted in FIGS. 2D-F, the value of p is 1, resulting from the reaction of BOC-blocked di-ethanolamine with intermediate II-a or II-b to generate intermediates II-c or II-d, respectively. Compounds in which p is 2 to 4 can be synthesized by substituting the appropriately sized BOC-blocked dialkylamino alcohol; that is, 3,3′-azanediylbis(propan-1-ol), 4,4′-azanediylbis(butan-1-ol), and 5,5′-azanediylbis(pentan-1-ol), respectively.

In the synthetic scheme depicted in FIGS. 2D-F, the value of n is 1 resulting from the reaction of intermediate II-i with the sodium salt of 3-dimethylamino-1-propanol, N,N-dimethyl-1,3-propanediamine, N,N,N′-trimethyl-1,3-propanediamine to generate Compounds A-5 to A-7, respectively, in which Y is O, NH, or N—CH₃, respectively. Compounds in which n is 0 or 2 to 4 can be synthesized by substituting the propanediamine moiety with an analogous C₂, C₄, C₅, or C₆ moiety. Compound A-8, in which Y is CH₂, is obtained by reacting a salt of intermediate II-g with 5-dimethylamino-pentanoic acid. Compounds in which n is 0 or 2 to 4 can be synthesized by substituting the pentanoic acid moiety with an analogous C₄, C₆, C₇, or C₈ moiety.

In the synthetic scheme depicted in FIGS. 2D-F, R is C₉, resulting from the use of decanoic acid in the conversion of intermediate II-e to intermediate II-f. Substitution of -oic acids of the corresponding chain length and structure can be used to obtain R of C₆-C₈ or C₁₀-C₁₈, as appropriate.

In the synthetic scheme depicted in FIGS. 2D-F, X is N(CH₃)₂. Compounds according to Formula 2 having alternative definitions of X can be synthesized by reacting alternative head group pieces from Tables 1-3 with II-i to obtain analogues of Compounds A-5 to A-7, respectively, or reacting alternative head group pieces from Table 4 with II-g to obtain analogues of Compound A-8, as disclosed in Example 2 (below). Synthesis of head group pieces not previously disclosed in the art can be made analogously to their shorter congeners or, for polyethylene glycol-containing head group pieces, made according to the synthetic schemes shown in FIGS. 4A-B and disclosed in Example 4, or as described in Examples 25-32, (below).

To synthesize intermediate II-f and its analogs with different R and/or p, that is an intermediate with the structure:

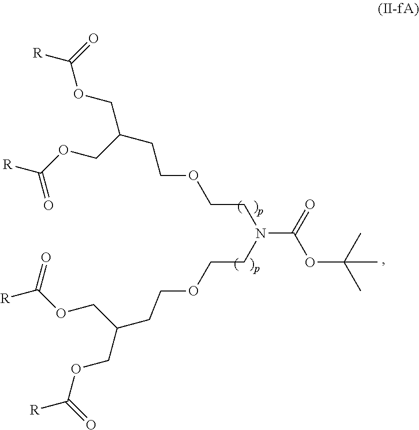

wherein p is an integer from 1-4 and R is defined as for Formula 2, the sodium salt of BOC-blocked di-ethanolamine, or the appropriately sized dialkylamino alcohol for p=2 to 4, is reacted with 5-(2-bromoethyl)-2,2-dimethyl-1,3-dioxane, II-a, or 5-(2-bromoethyl)-2-phenyl-1,3-dioxane, II-b, in DMF leading to II-c and II-d, respectively (FIG. 2D), or their analogues with p=2-4 (FIG. 2A). II-c and its analogues can be deblocked with mild acid in the presence of PPTs in MeOH to give diol II-e (FIG. 2D) and its analogues with p=2 to 4 (FIG. 2A). Alternatively, the benzylidene acetal II-d and its analogues can be deblocked with hydrogen and Pd/C in ethyl acetate to also lead to II-e (FIG. 2D), or its analogues with p=2-4 (FIG. 2A). Coupling of II-e with the appropriate carboxylic acid for the desired R in the presence of EDC-HCl and DMAP in dichloromethane leads to II-f (FIG. 2D) or its analogue with different R and/or p (FIG. 2A).

To synthesize an intermediate II-h and its analogs with different R and/or p, that is an intermediate with the structure:

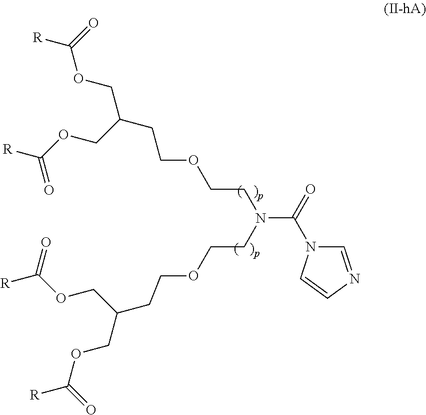

wherein p is an integer from 1-4 and R is defined as for Formula 2, an intermediate with a structure of II-fA is treated with TFA in dichloromethane to remove the BOC blocking group to afford the amine salt II-g (FIG. 2D) or its analogues with different R and/or p (FIG. 2A). The amine salt II-g or its analogues is reacted with carbonyl diimidazole and triethylamine in dichloromethane to yield the acylimidazole II-h or its analogues II-hA (FIG. 2A).

To complete the syntheses of compounds A-5 to A-7 and their analogues with different R and/or p, the needed reactive intermediate is obtained by the reaction of an intermediate with the structure of II-hA with methyl triflate to produce acyl-imidazolium II-i (FIG. 2E) or an analogue thereof with different R and/or p (FIG. 2B). The acyl-imidazolium intermediate is then reacted with 3-dimethylamino-1-propanol in the presence triethylamine, to provide Compound A-5 (FIG. 2E) or analogues with different R and/or p (FIG. 2B); with N,N-dimethyl-1,3-propanediamine and triethylamine to provide Compound A-6 (FIG. 2E) or analogues with different R and/or p (FIG. 2B); or with N,N,N′-trimethyl-1,3-propanediamine and triethylamine to provide Compound A-7 (FIG. 2E), in each case in dichloromethane, or analogues with different R and/or p (FIG. 2B).

To complete the synthesis of Compound A-8 or their analogues with different R and/or p, the salt II-g (FIG. 2F), or its analogs with different R and/or p (FIG. 2C), is reacted with 4-dimethylamino-butanoic acid in the presence of EDC-HCl, DMAP, and triethylamine, in dichloromethane, to provide Compound A-8 (FIG. 2F)) or analogues thereof with different R and/or p (FIG. 2C).

The reagent substitutions used to obtain analogues of Compounds A-5 to A-8 with different X, Y, and/or n are described above and are applicable to the foregoing syntheses for obtaining analogues of Compounds A-5 to A-8 with different R and/or p.

In certain aspects, the present disclosure provides a method of synthesizing an ionizable cationic lipid of Formula 3. In some embodiments, the method comprises converting an intermediate having a structure of III-cA to the ionizable cationic lipid of Formula 3. In some embodiments, the method further comprises synthesizing the intermediate having a structure of III-cA.

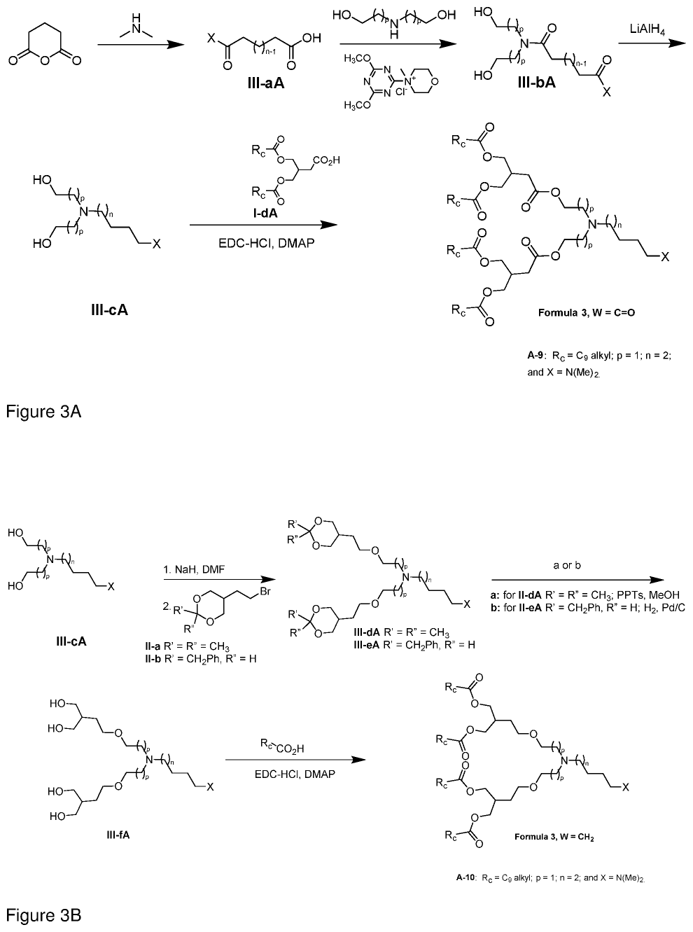

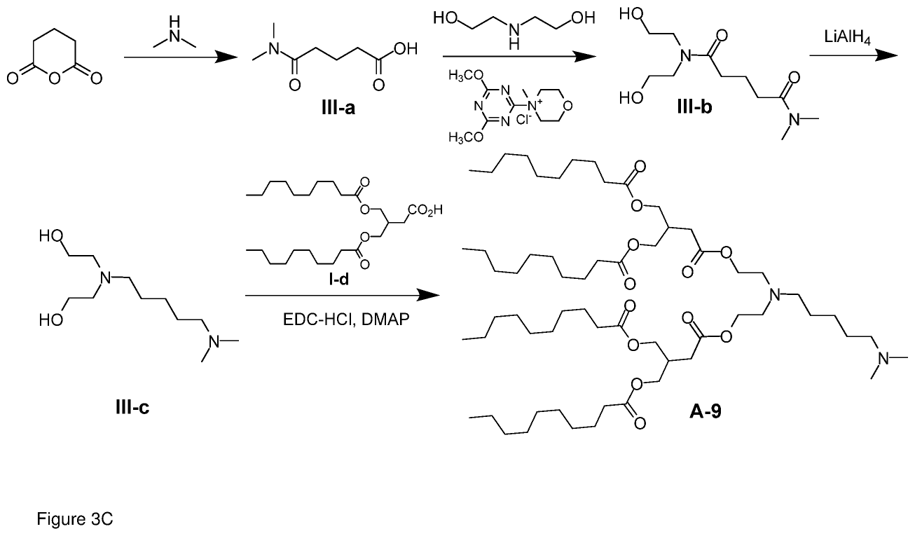

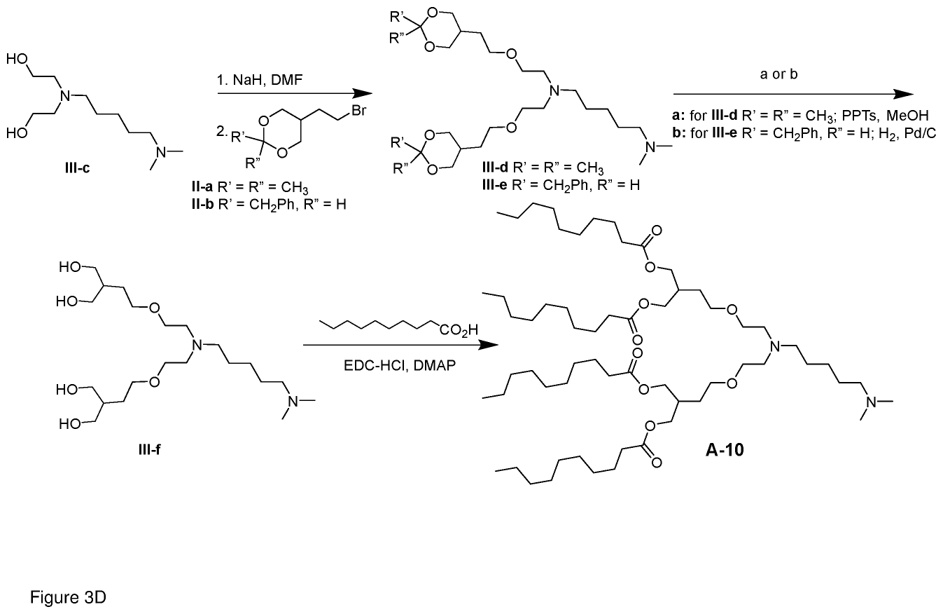

In certain embodiments of the synthesis method of the ionizable cationic lipid of Formula 3, W═C═O, and the method further comprises reacting III-cA with I-dA to provide the ionizable cationic lipid of Formula 3. See, e.g., FIG. 3A.

In certain embodiments of the synthesis method of the ionizable cationic lipid of Formula 3, W═CH₂, and the method further comprises converting III-cA to III-fA, and III-fA reacting with R—COOH to provide the ionizable cationic lipid of Formula 3. See, e.g., FIG. 3B. In certain embodiments, the method further comprises preparing a derivative of III-fA wherein the hydroxyl groups of III-fA are protected (i.e., the OH-protected III-fA). For example, as shown in FIG. 3B, the OH-protected III-fA can be III-dA which can be prepared by reacting the sodium salt of III-cA with II-a. In another example, as shown in FIG. 3B, the OH-protected III-fA can be III-eA which can be prepared by reacting the sodium salt of III-cA with II-b.

In certain embodiments, III-cA is prepared by reduction of carbonyl groups of III-bA, e.g., by LiAlH₄ as shown in FIG. 3A. In certain embodiments, III-bA is prepared by reacting III-aA with HN((CH₂)ₚ—CH₂OH)₂. In certain embodiments, reaction of III-aA and HN((CH₂)ₚ—CH₂OH)₂ is in the presence of 4-(4,6-dimethoxy-1,3,5-triazin-2-yl)-4-methylmorpholinium chloride, e.g., as shown in FIG. 3A. An embodiment of III-aA, wherein n=2, can be prepared by reacting glutaric anhydride with dimethylamine, e.g., as shown in FIG. 3A.

In some embodiments, the method of synthesizing an ionizable cationic lipid of Formula 3 proceeds according to the synthetic scheme of FIGS. 3A-D. In some embodiments, the method is a method of synthesizing an ionizable cationic lipid of Formula 3a. In some instances, the method is a method of synthesizing Compound A-9 or Compound A-10. In some embodiments, the method comprises only a single step, or subset of steps, depicted in FIGS. 3A-D.

Further aspects are intermediates III-aA, III-bA, III-cA, III-dA, III-eA, III-fA, III-d, III-e, and III-f of the synthetic scheme of FIGS. 3A-D and methods of synthesizing each of intermediates, III-d, III-e, and III-f. Further embodiments relate to analogues of the intermediates III-d, III-e, and III-f appropriate to final products with differing X, n, or p.

In further aspects, provided is a method of synthesizing an intermediate of the synthetic scheme of FIGS. 3C-D, wherein the intermediate is III-d, III-e, or III-f. In some embodiments, the method specifies only a final step to generate the intermediate as depicted in FIGS. 3C-D. In other embodiments, the method specifies all or a subset of the steps as depicted in FIGS. 3C-D to reach the intermediate.

In the synthetic scheme depicted in FIGS. 3C-D, the value of p is 1, resulting from the reaction of glutaric anhydride with dimethyl amine to form III-a which reacts with di-ethanolamine. Compounds in which p is 2 to 4 can be synthesized by substituting the appropriately sized dialkylamino alcohol; that is, 3,3′-azanediylbis(propan-1-ol), 4,4′-azanediylbis(butan-1-ol), and 5,5′-azanediylbis(pentan-1-ol), respectively.

In the synthetic scheme depicted in FIGS. 3C-D, the value of n is 2 resulting from the coupling of intermediate III-a with diethanolamine (4-(4,6-dimethoxy-1,3,5-triazin-2-yl)-4-methylmorpholinium chloride), and subsequent reduction. Compounds in which n is 0 to 1 or 3 to 4 can be synthesized by substituting malonic acid, maleic anhydride, 1,6-hexanedioic acid, 1,7-heptanedioic acid in the coupling reaction with dimethyl amine and subsequent addition of the amide-acid with the amino alcohol.

W is C═O in the synthesis of Compound A-9 depicted in FIG. 3C. W is CH₂ in the synthesis of Compound A-10 depicted in FIG. 3D.

In the synthetic scheme depicted in FIGS. 3C-D, R_{c} is C₉, resulting from the use of decanoic acid in the conversion of intermediate III-c or III-f to Compound 9 or 10, respectively. Substitution of -oic acids of the corresponding chain length and structure can be used to obtain R_{c} of C₆-C₈ or C₁₀-C₂₀, as appropriate.

In the synthetic scheme depicted in FIGS. 3C-D, X is N(CH₃)₂. Compounds according to Formula 3 having alternative definitions of X can be synthesized by reacting alternative head group pieces from Table 4 (instead of III-a) with diethanolamine to obtain analogues of Compounds A-9 and A-10, as disclosed in Example 3 (below). Synthesis of head group pieces not previously disclosed in the art can be made analogously to their shorter congeners or, for polyethylene glycol-containing head group pieces, made according to the synthetic schemes shown in FIGS. 4A-B and disclosed in Example 4, or as described in Examples 25-32, (below).

To complete the syntheses of Compound A-9 and its analogues with different R and p, as defined for Formula 3, first glutaric anhydride is reacted with dimethylamine in THF to give 5-(dimethylamino)-5-oxopentanoic acid III-a. The coupling of III-a with diethanolamine, or the appropriately sized dialkylamino alcohol for p=2 to 4, in the presence of (4-(4,6-Dimethoxy-1,3,5-triazin-2-yl)-4-methylmorpholinium chloride) gives N1,N1-bis(2-hydroxyethyl)-N5,N5-dimethyl-pentanediamide III-b (FIG. 3C) or analogues thereof (FIG. 3A), and reduction with LiAlH₄ in THF provides diol III-c (FIG. 3C) or analogues thereof with p=2 to 4 (FIG. 3A). Intermediate I-d or its analogues with different R, the synthesis of which is disclosed above, is then coupled with III-c in dichloromethane to afford Compound A-9 (FIG. 3C) and its analogues with different R_{c} and/or p (FIG. 3A).

To complete the syntheses of Compound A-10 and its analogues with different R and p, as defined for Formula 3, the sodium salt of III-c, or analogues thereof with p=2 to 4, is reacted with either bromide II-a or II-b in the presence of NaH in DMF to give the diamines III-d and III-e (FIG. 3D) or analogues thereof with p=2 to 4 (FIG. 3B). Deprotection of III-d with PPTs in methanol or deprotection of III-e with hydrogen and Pd/C in ethyl acetate provides tetraol III-f or analogues thereof with p=2 to 4. The coupling of tetraol III-f with decanoic acid, or other carboxylic acids appropriate to generate analogues with different R, in the presence of EDC-HCl and DMAP in dichloromethane leads to Compound A-10 (FIG. 3D) or its analogues with different R and/or p (FIG. 3B).

The reagent substitutions used to obtain analogues of Compounds A-9 to A-10 with different X and/or n are described above and are applicable to the foregoing syntheses for obtaining analogues of Compounds A-9 to A-10 with different R and/or p.

The syntheses are described using specific solvents, but in all cases alternative solvents will be known to the person of skill in the art. THF can be substituted, for example, without limitation, with DMF, diethyl ether, methyl t-butyl ether, dioxane, or 2-methyl THF. Ethyl acetate can be substituted with, for example, without limitation, isopropyl acetate, THF, 2-methyl THF, dioxane, or methyl t-butyl ether. Dichloromethane can be substituted with, for example, without limitation, ethyl acetate, isopropyl acetate, THF, methyl t-butyl ether, 2-methyl THF, dioxane, or heptane. Methanol can be substituted with, for example, without limitation, ethanol, or aqueous THF.

### Lipid Nanoparticles (LNPs) and Targeted LNPs (tLNPs)

As used herein, “lipid nanoparticle” (LNP) means a solid particle, as distinct from a liposome having an aqueous lumen. The core of a LNP, like the lumen of a liposome, is surrounded by a layer of lipid that may be, but is not necessarily, a continuous lipid monolayer, a bilayer as found in a liposome, or multi-layer having three or more lipid layers.

In certain aspects, the present disclosure provides a lipid nanoparticle (LNP) comprising an ionizable cationic lipid of Formula 1, Formula 2, or Formula 3, or a combination thereof. In some embodiments, an LNP comprises an ionizable cationic lipid of Formula 1, Formula 2, or Formula 3, or a combination thereof, and a phospholipid, a sterol, a co-lipid, or a PEGylated lipid, or a combination thereof. In certain embodiments, the PEG-lipids are not functionalized PEG-lipids. In certain embodiments, the LNP comprises at least one PEG-lipid that is functionalized and at least one that is not.

In further aspects, the present disclosure provides a targeted lipid nanoparticle (tLNP) comprising an ionizable cationic lipid of Formula 1, Formula 2, or Formula 3, or a combination thereof. In some embodiments, the aforementioned tLNP may further comprise one or more of a phospholipid, a sterol, a co-lipid, and a PEG-lipid, or a combination thereof, and a functionalized PEG-lipid. As used herein, “functionalized PEG-lipid” refers to a PEG-lipid in which the PEG moiety has been derivatized with a chemically reactive group that can be used for conjugating a targeting moiety to the PEG-lipid. The functionalized PEG-lipid can be reacted with a binding moiety after the LNP is formed, so that the binding moiety is conjugated to the PEG portion of the lipid. The conjugated binding moiety can thus serve as a targeting moiety for the tLNP.

With respect to LNPs or tLNPs of this disclosure, in various embodiments, a phospholipid comprises dioleoylphosphatidyl ethanolamine (DOPE), dimyristoylphosphatidyl choline (DMPC), distearoylphosphatidylcholine (DSPC), dimyristoylphosphatidyl glycerol (DMPG), dipalmitoyl phosphatidylcholine (DPPC), or 1,2-diarachidoyl-sn-glycero-3-phosphocholine (DAPC), or a combination thereof. Phospholipids can contribute to formation of a membrane, whether monolayer, bilayer, or multi-layer, surrounding the core of the LNP or tLNP. Additionally, phospholipids such as DSPC, DMPC, DPPC, DAPC impart stability and rigidity to membrane structure. Phospholipids, such as DOPE, impart fusogenicity. Further phospholipids, such as DMPG, which attains negative charge at physiologic pH, facilitates charge modulation. Thus, phospholipids constitute means for facilitating membrane formation, means for imparting membrane stability and rigidity, means for imparting fusogenicity, and means for charge modulation.

With respect to LNPs or tLNPs of this disclosure, in various embodiments, a sterol is cholesterol, 20-hydroxycholesterol, 22-hydroxycholesterol, or a phytosterol. In further embodiments the phytosterol comprises campesterol, sitosterol, or stigmasterol, or combinations thereof. In preferred embodiments, the cholesterol is not animal-sourced but is obtained by synthesis using a plant sterol as a starting point. LNPs incorporating C-24 alkyl (such as methyl or ethyl) phytosterols have been reported to provide enhanced gene transfection. The length of the alkyl tail, the flexibility of the sterol ring, and polarity related to a retain C-3 —OH group are important to obtaining high transfection efficiency. While β-sitosterol and stigmasterol performed well, vitamin D2, D3 and calcipotriol, (analogs lacking intact body of cholesterol) and betulin, lupeol ursolic acid and olenolic acid (comprising a 5th ring) should be avoided. Sterols serve to fill space between other lipids in the LNP or tLNP and influence LNP or tLNP shape. Sterols also control fluidity of lipid compositions, reducing temperature dependence. Thus, sterols such as cholesterol, 20-hydroxycholesterol, 22-hydroxycholesterol, campesterol, fucosterol, β-sitosterol, and stigmasterol constitute means for controlling LNP shape and fluidity or sterol means for increasing transfection efficiency.

With respect to LNPs or tLNPs of this disclosure, in some embodiments, a co-lipid is absent or comprises an ionizable lipid, anionic or cationic. A co-lipid can be used to adjust various properties of an LNP or tLNP, such as surface charge, fluidity, rigidity, size, stability, etc. In some embodiments, a co-lipid is an ionizable lipid, such as cholesterol hemisuccinate (CHEMS) or an ionizable lipid of this disclosure. In some embodiments, a co-lipid is a charged lipid, such as a quaternary ammonium headgroup containing lipid. In some instances, a quaternary ammonium headgroup containing lipid comprises 1,2-dioleoyl-3-trimethylammonium propane (DOTAP), N-(1-(2,3-dioleyloxy)propyl)-N,N,N-trimethylammonium (DOTMA), or 3β-(N—(N′,N′-Dimethylaminoethane)carbamoyl)cholesterol (DC-Chol), or combinations thereof. In certain embodiments, these compounds a chloride, bromide, mesylate, or tosylate salt.

When the disclosed ionizable lipids of Formulas 1, 2, and 3 have a measured pKa between 6 and 7, they can contribute substantial endosomal release activity to an LNP or tLNP containing the ionizable lipid. More acidic or basic ionizable lipids of Formulas 1, 2, and 3 can contribute surface charge and thus serve as a co-lipid as described immediately above. In such cases, it can be advantageous to incorporate another lipid with fusogenic activity into a LNP or tLNP of this disclosure. Surface charge is known to influence the tissue tropism of LNPs or tLNPs; for example, positively charged LNPs or tLNPs have shown a tropism for spleen and lung.

With respect to a LNP or tLNP of this disclosure, in some embodiments, a PEG-lipid (that is, a lipid conjugated to a polyethylene glycol (PEG)) is a C₁₄-C₂₀ lipid conjugated with a PEG. PEG-lipids with fatty acid chain lengths less than C₁₄ are too rapidly lost from the (t)LNP while those with chain lengths greater than C₂₀ are prone to difficulties with formulation. In some embodiments, a PEG is of 500-5000 or 1000-5000 Da molecular weight (MW). In some embodiments, the PEG unit has a MW of 2000 Da. In some instances, the MW2000 PEG-lipid comprises DMG-PEG2000 (1,2-dimyristoyl-glycero-3-methoxypolyethylene glycol-2000), DPG-PEG2000 (1,2-dipalmitoyl-glycero-3-methoxypolyethylene glycol-2000), DSG-PEG2000 (1,2-distearoyl-glycero-3-methoxypolyethylene glycol-2000), DOG-PEG2000 (1,2-dioleoyl-glycero-3-methoxypolyethylene glycol-2000), DMPE-PEG200 (1,2-dimyristoyl-glycero-3-phosphoethanolamine-3-methoxypolyethylene glycol-2000), DPPE-PEG2000 (1,2-dipalmitoyl-glycero-3-phosphoethanolamine-3-methoxypolyethylene glycol-2000), DSPE-PEG2000 (1,2-distearoyl-glycero-3-phosphoethanolamine-3-methoxypolyethylene glycol-2000), DOPE-PEG2000 (1,2-dioleoyl-glycero-3-phosphoethanolamine-3-methoxypolyethylene glycol-2000), or combinations thereof. In some embodiments, the PEG unit has a MW of 2000 Da. In some instances, the MW2000 PEG-lipid comprises DMrG-PEG2000 (1,2-dimyristoyl-rac-glycero-3-methoxypolyethylene glycol-2000), DPrG-PEG2000 (1,2-dipalmitoyl-rac-glycero-3-methoxypolyethylene glycol-2000), DSrG-PEG2000 (1,2-distearoyl-rac-glycero-3-methoxypolyethylene glycol-2000), DOrG-PEG2000 (1,2-dioleoyl-glycero-3-methoxypolyethylene-rac-glycol-2000), DMPEr-PEG200 (1,2-dimyristoyl-rac-glycero-3-phosphoethanolamine-3-methoxypolyethylene glycol-2000), DPPEr-PEG2000 (1,2-dipalmitoyl-rac-glycero-3-phosphoethanolamine-3-methoxypolyethylene glycol-2000), DSPEr-PEG2000 (1,2-distearoyl-rac-glycero-3-phosphoethanolamine-3-methoxypolyethylene glycol-2000), DOPEr-PEG2000 (1,2-dioleoyl-rac-glycero-3-phosphoethanolamine-3-methoxypolyethylene glycol-2000), or combinations thereof. Alternatively, optically pure antipodes of the glycerol portion can be employed, that is, the glycerol portion is homochiral. As used herein with respect to glycerol moieties, optically pure means ≥95% of a single enantiomer (D or L). In some embodiments, the enantiomeric excess is ≥98%. In some embodiments, the enantiomeric excess is ≥99%. Additional PEG-lipids, including achiral PEG-lipids built on a symmetric dihydroxyacetone scaffold, a symmetric 2-(hydroxymethyl)butane-1,4-diol, or a symmetric glycerol scaffold, are disclosed in U.S. Provisional Application No. 63/362,502, filed on Apr. 5, 2022, and PCT application filed on Apr. 5, 2023 (Atty. Docket No. 146758-8002. WO00), both entitled PEG-Lipids and Lipid Nanoparticles, which are incorporated by reference in their entirety.

A PEG-moiety provides a hydrophilic surface on the LNP, inhibiting aggregation or merging of LNP, thus contributing to their stability and reducing polydispersity. Additionally, a PEG moiety may impede binding by the LNP, including binding to plasma proteins. These plasma proteins include apoE which is understood to mediate uptake of LNP by the liver so that inhibition of binding can lead to an increase in the proportion of LNP reaching other tissues. These plasma proteins also include opsonins so that inhibition of binding reduces recognition by the reticuloendothelial system. The PEG-moiety can also be functionalized to serve as an attachment point for a targeting moiety. Conjugating a cell- or tissue-specific binding moiety to the PEG-moiety enables a tLNP to avoid the liver and bind to its target tissue or cell type, greatly increasing the proportion of LNP that reaches the targeted tissue or cell type. PEG-lipid can thus serve as means for inhibiting LNP binding, and PEG-lipid conjugated to a binding moiety can serve as means for LNP-targeting.

In some embodiments, a “binding moiety” or “targeting moiety” refers to a protein, polypeptide, oligopeptide, peptide, carbohydrate, nucleic acid, or combination thereof that is capable of specifically binding to a target or multiple targets. A binding domain includes any naturally occurring, synthetic, semi-synthetic, or recombinantly produced binding partner for a biological molecule or another target of interest. Exemplary binding moieties of this disclosure include an antibody, a Fab′, F(ab′)₂, Fab, Fv, rIgG, scFv, hcAbs (heavy chain antibodies), a single domain antibody, VHH, VNAR, sdAbs, nanobody, receptor ectodomains or ligand-binding portions thereof, or ligands (e.g., cytokines, chemokines). A “Fab” (fragment antigen binding) is the part of an antibody that binds to antigens and includes the variable region and CH₁ of the heavy chain linked to the light chain via an inter-chain disulfide bond. A variety of assays are known for identifying binding moieties of the present disclosure that specifically bind a particular target, including Western blot, ELISA, and Biacore® analysis. A binding moiety, such as a binding moiety comprising immunoglobulin light and heavy chain variable domains (e.g., scFv), can be incorporated into a variety of protein scaffolds or structures as described herein, such as an antibody or an antigen binding fragment thereof, a scFv-Fc fusion protein, or a fusion protein comprising two or more of such immunoglobulin binding domains.

An antibody or other binding moiety (or a fusion protein thereof) “specifically binds” a target if it binds the target with an affinity or Ka (i.e., an equilibrium association constant of a particular binding interaction with units of 1/M) equal to or greater than 10⁵ M⁻¹, while not significantly binding other components present in a test sample. Binding domains (or fusion proteins thereof) may be classified as “high affinity” binding domains (or fusion proteins thereof) and “low affinity” binding domains (or fusion proteins thereof). “High affinity” binding domains refer to those binding domains with a Ka of at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹, preferably at least 10⁸ M⁻¹ or at least 10⁹ M⁻¹. “Low affinity” binding domains refer to those binding domains with a Ka of up to 10⁸ M⁻¹, up to 10⁷ M⁻¹, up to 10⁶ M⁻¹, up to 10⁵ M⁻¹. Alternatively, affinity may be defined as an equilibrium dissociation constant (Kd) of a particular binding interaction with units of M (e.g., 10⁻⁵ M to 10⁻¹³ M). Affinities of binding domain polypeptides and fusion proteins according to the present disclosure can be readily determined using conventional techniques (see, e.g., Scatchard et al., Ann. N.Y. Acad. Sci. 51:660, 1949; and U.S. Pat. Nos. 5,283,173, 5,468,614, or the equivalent).

Some embodiments of the disclosed ionizable cationic lipids have head groups with small (<250 Da) PEG moieties. These lipids are not what is meant by the term PEG-lipid as used herein. These small PEG moieties are generally too small to impede binding to a similar extent as the larger PEG moieties of the PEG-lipids disclosed above, though they will impact the lipophilicity of ionizable cationic lipid. Moreover, the PEG-lipids are understood to be primarily located in an exterior facing lamella whereas much of the ionizable cationic lipid is in the interior of the LNP.

In various embodiments, a binding moiety of a tLNP comprises an antigen binding domain of an antibody, an antigen, a ligand-binding domain of a receptor, or a receptor ligand. In some embodiments, the binding moiety comprising an antigen binding domain of an antibody comprises a complete antibody, an F(ab)2, an Fab, a minibody, a single-chain Fv (scFv), a diabody, a VH domain, or a nanobody, such as a VHH or single domain antibody. In some embodiments, the receptor ligand is a carbohydrate, for example, a carbohydrate comprising terminal galactose or N-acetylgalactosamine units, which are bound by the asialoglycoprotein receptor. These binding moieties constitute means for LNP targeting. Some embodiments specifically include one or more of these binding moieties. Other embodiments specifically exclude one or more of these binding moieties.

As used herein, “antibody” refers to a protein comprising an immunoglobulin domain having hypervariable regions determining the specificity with which the antibody binds antigen; so-called complementarity determining regions (CDRs). The term antibody can thus refer to intact or whole antibodies as well as antibody fragments and constructs comprising an antigen binding portion of a whole antibody. While the canonical natural antibody has a pair of heavy and light chains, camelids (camels, alpacas, llamas, etc.) produce antibodies with both the canonical structure and antibodies comprising only heavy chains. The variable region of the camelid heavy chain only antibody has a distinct structure with a lengthened CDR3 referred to as VHH or, when produced as a fragment, a nanobody. Antigen binding fragments and constructs of antibodies include F(ab)₂, F(ab), minibodies, Fv, single-chain Fv (scFv), diabodies, and VH. Such elements may be combined to produce bi- and multi-specific reagents, such as BiTEs. The term “monoclonal antibody” arose out of hybridoma technology but is now used to refer to any singular molecular species of antibody regardless of how it was originated or produced. Antibodies can be obtained through immunization, selection from a naïve or immunized library (for example, by phage display), alteration of an isolated antibody-encoding sequence, or any combination thereof. Numerous antibodies that could be used as binding moieties are known in the art. An excellent source of information about antibodies for which an International Non-proprietary Name (INN) has been proposed or recommended, including sequence information, is Wilkinson & Hale, MAbs 14(1):2123299, 2022, including its Supplementary Tables, which is incorporated by reference herein for all that it teaches about individual antibodies and the various antibody formats that can be constructed. U.S. Pat. No. 11,326,182 and especially its Table 9 Cancer, Inflammation and Immune System Antibodies, is a source of sequence and other information for a wide range of antibodies including many that do not have an INN and is incorporated herein by reference for all that it teaches about individual antibodies.

A functionalized PEG-lipid of a tLNP comprises one or more fatty acid tails, each that is no shorter than C₁₆ nor longer than C₂₀ for straight-chain fatty acids. For branched chain fatty acids, tails no shorter than C₁₄ fatty acids nor longer than C₂₀ are acceptable. In some embodiments, fatty acid tails are C₁₆. In some embodiments, the fatty acid tails are C₁₈. In some embodiments, the functionalized PEG-lipid comprises a dipalmitoyl lipid. In some embodiments, the functionalized PEG-lipid comprises a distearoyl lipid. The fatty acid tails serve as means to anchor the PEG-lipid in the tLNP to reduce or eliminate shedding of the PEG-lipid from the tLNP. This is a useful property for the PEG-lipid whether or not it is functionalized but has greater significance for the functionalized PEG-lipid as it will have a targeting moiety attached to it and the targeting function could be impaired if the PEG-lipid (with the conjugated binding moiety) were shed from the tLNP.

Any suitable chemistry may be used to conjugate the binding moiety to the PEG of the PEG-lipid, including maleimide (see Parhiz et al., Journal of Controlled Release 291:106-115, 2018) and click (see Kolb et al., Angewandte Chemie International Edition 40(11):2004-2021, 2001; and Evans, Australian Journal of Chemistry 60(6):384-395, 2007) chemistries. Reagents for such reactions include lipid-PEG-maleimide, lipid-PEG-cysteine, lipid-PEG-alkyne, lipid, PEG-dibenzocyclooctyne (DBCO), and lipid-PEG-azide. Further conjugations reactions make use of lipid-PEG-bromo maleimide, lipid-PEG-alkylnoic amide, PEG-alkynoic imide, and lipid-PEG-alkyne reactions, as disclosed in U.S. Provisional Application No. 63/362,502, filed on Apr. 5, 2022, and PCT application filed on Apr. 5, 2023 (Atty. Docket No. 146758-8002. WO00), both entitled PEG-Lipids and Lipid Nanoparticles, which are incorporated by reference in their entirety. On the binding moiety side of the reaction one can use an existing cysteine sulfhydryl, or derivatize the protein by adding a sulfur containing carboxylic acid, for example, to the epsilon amino of a lysine to react with a maleimide, bromomaleimide, alkylnoic amide, or alkynoic imide. Alternatively, one can add an alkyne to a sulfhydryl or an epsilon amino of a lysine to participate in a click chemistry reaction.

With respect to LNPs or tLNPs of this disclosure, in some embodiments, the molar ratio of the lipids is about 40 to about 60 mol % ionizable cationic lipid. In some embodiments of the LNP or the tLNP, the molar ratio of the lipids is about 7 to about 30 mol % phospholipid. In some embodiments of the LNP or the tLNP, the molar ratio of the lipids is about 20 to about 45 mol % sterol. In some embodiments of the LNP or the tLNP, the molar ratio of the lipids is 1 to 30 mol % co-lipid. In some embodiments of the LNP or the tLNP, the molar ratio of the lipids is 0 to 5 mol % PEG-lipid. In some embodiments of the LNP or the tLNP, the molar ratio of the lipids is 0.1 to 5 mol % functionalized PEG-lipid. In some embodiments, the functionalized PEG-lipid is conjugated to a binding moiety.

Due to physiologic and manufacturing constraints LNP or tLNP for in vivo use, particles with a hydrodynamic diameter of about 50 to about 150 nm are desirable. Accordingly, in some embodiments, the LNP or tLNP has a hydrodynamic diameter of 50 to 150 nm and in some instances the hydrodynamic diameter is ≤120, ≤110, ≤100, or ≤90 nm. Uniformity of particle size is also desirable with a polydispersity index (PDI) of ≤0.2 (on a scale of 0 to 1) being acceptable. Both hydrodynamic diameter and polydispersity index are determined by dynamic light scattering (DLS). Particle diameter as assessed from cryo-transmission electron microscopy (Cryo-TEM) can be smaller than the DLS-determined value.

LNPs or tLNPs of this disclosure further comprise a nucleic acid. In various embodiments, a nucleic acid is an mRNA, a self-replicating RNA, a siRNA, a miRNA, DNA, a gene editing component (for example, a guide RNA, a tracr RNA, a sgRNA), a gene writing component, an mRNA encoding a gene or base editing protein, a zinc-finger nuclease, a Talen, a CRISPR nuclease, such as Cas9, a DNA molecule to be inserted or serve as a template for repair), and the like, or a combination thereof. In some embodiments, an mRNA encodes a chimeric antigen receptor (CAR). In other embodiments, an mRNA encodes a gene-editing or base-editing or gene writing protein. In some embodiments, a nucleic acid is a guide RNA. In some embodiments, an LNP or tLNP comprises both a gene- or base-editing or gene writing protein-encoding mRNA and one or more guide RNAs. CRISPR nucleases may have altered activity, for example, modifying the nuclease so that it is a nickase instead of making double-strand cuts or so that it binds the sequence specified by the guide RNA but has no enzymatic activity. Base-editing proteins are often fusion proteins comprising a deaminase domain and a sequence-specific DNA binding domain (such as an inactive CRISPR nuclease).

With respect to LNPs or tLNPs of this disclosure, in some embodiments, the ratio of total lipid to nucleic acid is about 10:1 to about 50:1 on a weight basis. In some embodiments, the ratio of total lipid to nucleic acid is about 10:1, about 20:1, about 30:1, or about 40:1 to about 50:1, or 10:1 to 20:1, 30:1, 40:1 or 50:1, or any range bound by a pair of these ratios.

In some aspects, the present disclosure provides a method of making a LNP or tLNP comprising mixing of an aqueous solution of a nucleic acid and an alcoholic solution of the lipids. In particular embodiments, the mixing is rapid. The aqueous solution is buffered at pH of about 3 to about 5, for example, without limitation, with citrate or acetate. In various embodiments, an alcohol can be ethanol, isopropanol, t-butanol, or a combination thereof. In some embodiments, the rapid mixing is accomplished by pumping the two solutions through a T-junction or with an impinging jet mixer. Microfluidic mixing through a staggered herringbone mixer (SHM) or a hydrodynamic mixer (microfluidic hydrodynamic focusing), microfluidic bifurcating mixers, and microfluidic baffle mixers can also be used. After the LNPs are formed they are diluted with buffer, for example phosphate, HEPES, or Tris, in a pH range of 6 to 8.5 to reduce the alcohol (ethanol) concentration, The diluted LNPs are purified either by dialysis or ultrafiltration or diafiltration using tangential flow filtration (TFF) against a buffer in a pH range of 6 to 8.5 (for example, phosphate, HEPES, or Tris) to remove the alcohol. Alternatively, one can use size exclusion chromatography. Once the alcohol is completely removed the buffer is exchanged with like buffer containing a cryoprotectant (for example, glycerol or a sugar such as sucrose, trehalose, or mannose). The LNPs are concentrated to a desired concentrated, followed by 0.2 μm filtration through, for example, a polyethersulfone (PES) or modified PES filter and filled into glass vials, stoppered, capped, and stored frozen. In alternative embodiments, a lyoprotectant is used and the LNP lyophilized for storage instead of as a frozen liquid. Further methodologies for making LNP can be found, for example, in US20200297634, US20130115274, and WO2017/048770, each of which is incorporated by references for all that they teach about the production of LNP.

One aspect is a method of making a tLNP comprising rapid mixing of an aqueous solution of a nucleic acid and an alcoholic solution of the lipids as disclosed for LNP. In some embodiments, the lipid mixture includes functionalized PEG-lipid, for later conjugation to a targeting moiety. As used herein, functionalized PEG-lipid refers to a PEG-lipid in which the PEG moiety has been derivatized with a chemically reactive group (such as, maleimide, NHO ester, Cys, azide, alkyne, and the like) that can be used for conjugating a targeting moiety to the PEG-lipid, and thus, to the LNP comprising the PEG-lipid. In other embodiments, the functionalized PEG-lipid is inserted into and LNP subsequent to initial formation of an LNP from other components. In either type of embodiment, the targeting moiety is conjugated to functionalized PEG-lipid after the functionalized PEG-lipid containing LNP is formed. Protocols for conjugation can be found, for example, in Parhiz et al. J. Controlled Release 291:106-115, 2018, and Tombacz et al., Molecular Therapy 29(11):3293-3304, 2021, each of which is incorporated by reference for all that it teaches about conjugation of PEG-lipids to binding moieties. Alternatively, the targeting moiety can be conjugated to the PEG-lipid prior to insertion into pre-formed LNP.

In certain embodiments of the preparation methods of tLNP, the method comprises:
i). forming an initial LNP by mixing all components of the tLNP except for the one or more functionalized PEG-lipids and the one or more targeting moieties;
ii). forming a pre-conjugation tLNP by mixing the initial LNP with the one or more functionalized PEG-lipids; and
iii). forming the tLNP by conjugating the pre-conjugation tLNP with the one or more targeting moieties.

In certain embodiments of the preparation methods of tLNP, the method comprises:
i). forming a pre-conjugation tLNP by mixing all components of the tLNP, including the one or more functionalized PEG-lipids, except for the one or more targeting moieties; and
ii). forming the tLNP by conjugating the pre-conjugation tLNP with the one or more targeting moieties.

In certain embodiments of the preparation methods of tLNP, the method comprises:
i). forming one or more conjugated functionalized PEG-lipids by conjugating the one or more functionalized PEG-lipids with the one or more targeting moieties; and
ii) forming the tLNP by mixing all components of the tLNP, including the one or more conjugated functionalized PEG-lipids.

In certain embodiments of the preparation methods of tLNP, the method comprises:
i). forming one or more conjugated functionalized PEG-lipids by conjugating the one or more functionalized PEG-lipids with the one or more targeting moieties;
ii) forming an LNP by mixing all components of the tLNP, except the one or more conjugated functionalized PEG-lipids; and
iii) forming the tLNP by mixing the initial LNP with the one or more conjugated functionalized PEG-lipids.

After the conjugation the tLNPs are purified by dialysis, tangential flow filtration, or size exclusion chromatography, and stored, as disclosed above for LNPs.

The encapsulation efficiency of the nucleic acid by the LNP or tLNP is typically determined with a nucleic acid binding fluorescent dye added to intact and lysed aliquots of the final LNP or tLNP preparation to determine the amounts of unencapsulated and total nucleic acid, respectively. Encapsulation efficiency is typically expressed as a percentage and calculated as 100×(T−U)/T where T is the total amount of nucleic acid and U is the amount of unencapsulated nucleic acid. In various embodiments, the encapsulation efficiency is 80%, 85%, 90%, or 95%.

In other aspects, disclosed herein are methods of delivering a nucleic acid into a cell comprising contacting the cell with LNP or tLNP of any of the forgoing aspects. In some embodiments the contacting takes place ex vivo. In some embodiments, the contacting takes place in vivo. In some instances, the in vivo contacting comprises intravenous, intramuscular, subcutaneous, intranodal or intralymphatic administration. In some embodiments, toxicity is confined (or largely confined) to grades of 0 or 1 or two, as discussed above.

The following examples are intended to illustrate various embodiments of the invention. As such, the specific embodiments discussed are not to be constructed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein to the extent that they are not inconsistent with the present disclosure.

### EXAMPLES

### Example 1: Synthesis of Compounds Having a Structure of Formula 1

Dihydroxy acetone can react with tert-butoxycarbonylmethylene)triphenylphosphorane to provide alkene I-a. Hydrogenation of I-a provides I-b and the coupling (EDC-HCl, DMAP) of I-b with decanoic acid results in tri-ester I-c. Hydrolysis of the t-butyl ester (CF₃CO₂H, CH₂Cl₂) results in a mono-acid I-d. Coupling of I-d with BOC-blocked di-ethanolamine affords I-e. BOC-removal (CF₃CO₂H, CH₂Cl₂) provides salt I-f (see FIG. 1D) which is converted to acyl-imidazolide 1-g upon reaction with carbonyl diimidazole Reaction of 1-g with methyl triflate produced acyl-imidazolium I-h, which is an intermediate to be converted to Compounds A-1 to A-3. The reaction of I-h with 3-dimethylamino-1-propanol, in the presence of base, leads to carbamate Compound A-1, the reaction of I-h with N,N-dimethyl-1,3-propanediamine provides NH-urea Compound A-2, and the reaction of I-h with N,N,N′-trimethyl-1,3-propanediamine leads to Compound A-3 (see FIG. 1E).

Compound A-4 can be obtained from the reaction of salt I-f with 5-dimethylamino-pentanoic acid (EDC-HCl, DMAP, Et₃N) (see FIG. 1F).

The headgroup in Compound A-1, that is, X in Formula 1 is derived from 3-dimethylamino-1-propanol. To obtain analogues of Compound A-1 with the disclosed alternative headgroups X and various values of n, the compounds of Table 1 may be used to substitute for 3-dimethylamino-1-propanol in the conversion of I-h.

TABLE 1
Alternative Headgroups of Compound A-1

| | |
|---|---|
| XR1 | 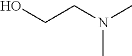 |
| XR2 | 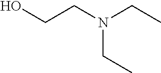 |
| XR3 | 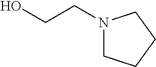 |
| XR4 | 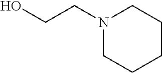 |
| XR5 | 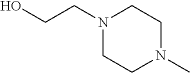 |
| XR6 | 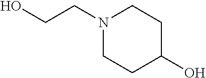 |
| XR7 | 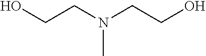 |
| XR8 | 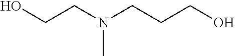 |
| XR9 | 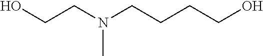 |
| XR10 | 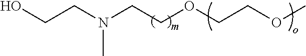 |
| XR11 | 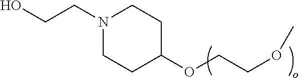 |
| XR12 | 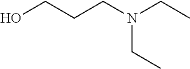 |
| XR13 | 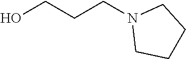 |
| XR14 | 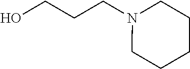 |
| XR15 | 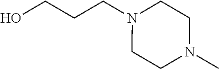 |
| XR16 | 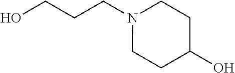 [SMILES: OCCCN1CCC(O)CC1] |
| XR17 | 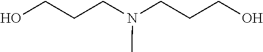 |
| XR18 | 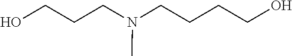 |
| XR19 | 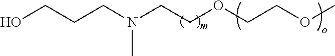 |
| XR20 | 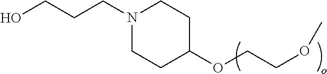 |
| XR21 | 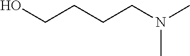 |
| XR22 | 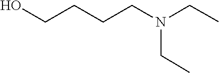 |
| XR23 | 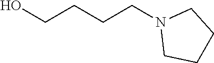 |
| XR24 | 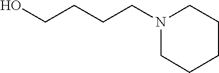 [SMILES: OCCCCN1CCCCC1] |
| XR25 | 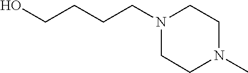 |
| XR26 | 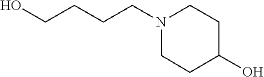 [SMILES: OCCCCN1CCC(O)CC1] |
| XR27 | 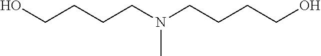 |
| XR28 | 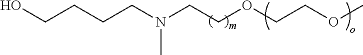 |
| XR29 | 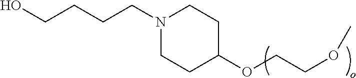 |
| XR30 | 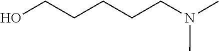 |
| XR31 | 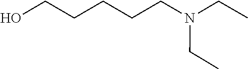 |
| XR32 | 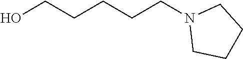 [SMILES: OCCCCCN1CCCC1] |
| XR33 | 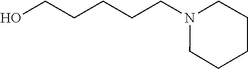 [SMILES: OCCCCCN1CCCCC1] |
| XR34 | 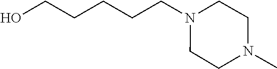 |
| XR35 | 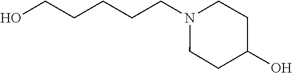 [SMILES: OCCCCCN1CCC(O)CC1] |
| XR36 | 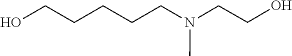 |
| XR37 | 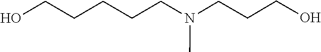 |
| XR38 | 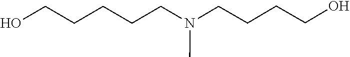 |
| XR39 | 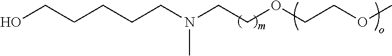 |
| XR40 | 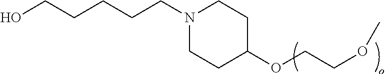 |
| XR41 | 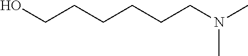 |
| XR42 | 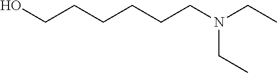 |
| XR43 | 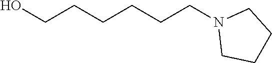 [SMILES: OCCCCCCN1CCCC1] |
| XR44 | 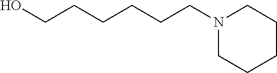 [SMILES: OCCCCCCN1CCCCC1] |
| XR45 | 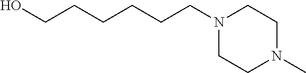 |
| XR46 | 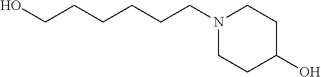 [SMILES: OCCCCCCN1CCC(O)CC1] |
| XR47 | 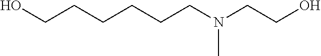 |
| XR48 | 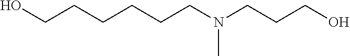 |
| XR49 | 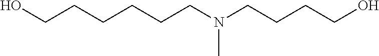 |
| XR50 | 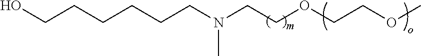 |
| XR51 | 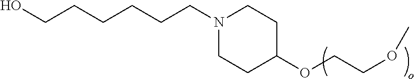 |

Reagents XR1-XR9, XR12-XR18, XR21-XR27, XR30-XR38, and XR41-49 are known in the art, as reported by the Chemical Abstract Society's SciFinder® with XR1-XR5, XR7, XR12-XR15, XR21-XR25, XR30-XR31, XR33, and XR41 being commercially available. The polyethylene glycol-containing reagents can be synthesized as described in Example 4, as shown below.

The headgroup in Compound A-2, that is, X in Formula 1 is derived from N,N-dimethyl-1,3-propanediamine. To obtain analogues of Compound A-2 with the disclosed alternative headgroups X and various values of n, the compounds of Table 2 may be used to substitute N,N-dimethyl-1,3-propanediamine in the conversion of I-h.

TABLE 2
Alternative Headgroups of Compound A-2

| | |
|---|---|
| XR52 | 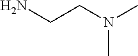 |
| XR53 | 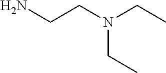 |
| XR54 | 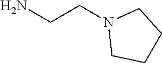 |
| XR55 | 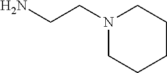 |
| XR56 | 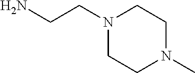 |
| XR57 | 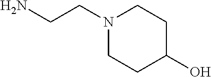 |
| XR58 | 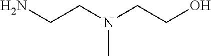 |
| XR59 | 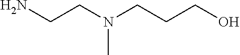 |
| XR60 | 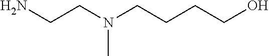 |
| XR61 | 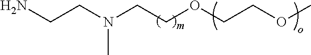 |
| XR62 | 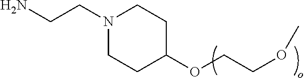 |
| XR63 | 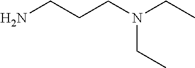 |
| XR64 | 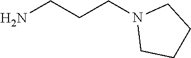 |
| XR65 | 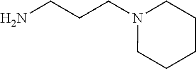 |
| XR66 | 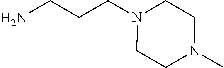 |
| XR67 | 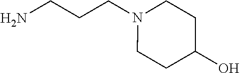 [SMILES: NCCCN1CCC(O)CC1] |
| XR68 | 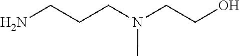 |
| XR69 | 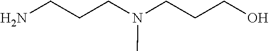 |
| XR70 | 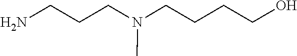 |
| XR71 | 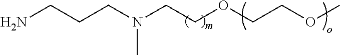 |
| XR72 | 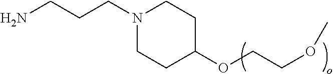 |
| XR73 | 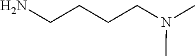 |
| XR74 | 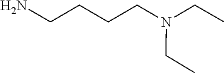 |
| XR75 | 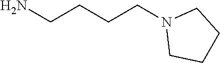 |
| XR76 | 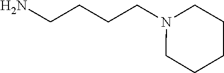 [SMILES: NCCCCN1CCCCC1] |
| XR77 | 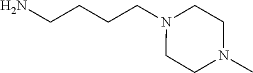 |
| XR78 | 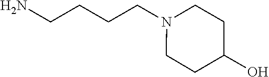 [SMILES: NCCCCN1CCC(O)CC1] |
| XR79 | 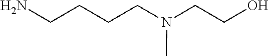 |
| XR80 | 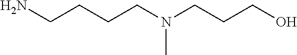 |
| XR81 | 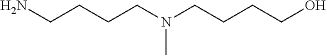 |
| XR82 | 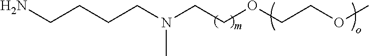 |
| XR83 | 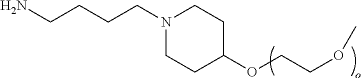 |
| XR84 | 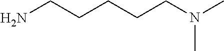 |
| XR85 | 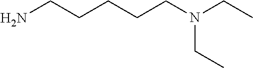 |
| XR86 | 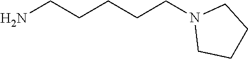 [SMILES: NCCCCCN1CCCC1] |
| XR87 | 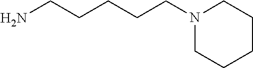 [SMILES: NCCCCCN1CCCCC1] |
| XR88 | 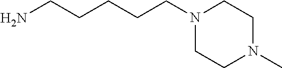 |
| XR89 | 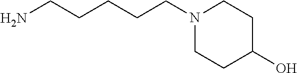 [SMILES: NCCCCCN1CCC(O)CC1] |
| RX90 | 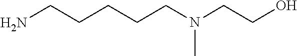 |
| XR91 | 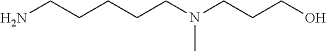 |
| XR92 | 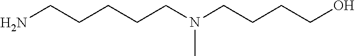 |
| XR93 | 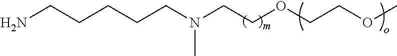 |
| XR94 | 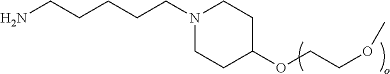 |
| XR95 | 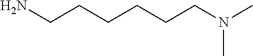 |
| XR96 | 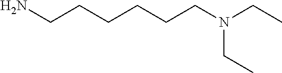 |
| XR97 | 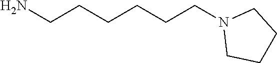 [SMILES: NCCCCCCN1CCCC1] |
| XR98 | 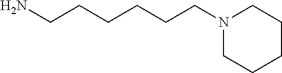 [SMILES: NCCCCCCN1CCCCC1] |
| XR99 | 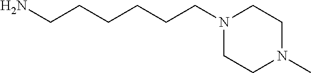 |
| XR100 | 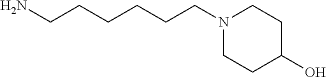 [SMILES: NCCCCCCN1CCC(O)CC1] |
| XR101 | 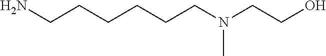 |
| XR102 | 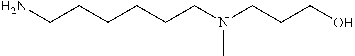 |
| XR103 | 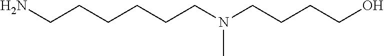 |
| XR104 | 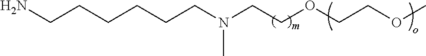 |
| XR105 | 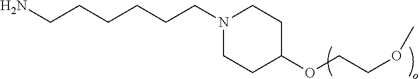 |

Reagents XR52-XR60, XR63-XR70, XR73-XR81, XR84-XR92, and XR95-XR103 are known in the art, as reported by the Chemical Abstract Society's SciFinder® with XR52-XR57, XR63-XR66, XR73-XR77, XR84, XR86-XR87, and XR95 being commercially available. The polyethylene glycol-containing reagents can be synthesized as described in Example 4, as shown below.

The headgroup in Compound A-3, that is, X in Formula 1 is derived from N,N,N′-trimethyl-1,3-propanediamine. To obtain analogues of Compound A-3 with the disclosed alternative headgroups X and various values of n, the compounds of Table 3 may be used to substitute N,N,N′-trimethyl-1,3-propanediamine in the conversion of I-h.

TABLE 3
Alternative Headgroups of Compound A-3

| | |
|---|---|
| XR106 | 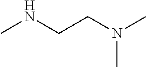 |
| XR107 | 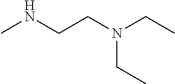 |
| XR108 | 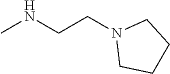 |
| XR109 | 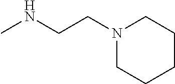 |
| XR110 | 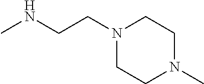 |
| XR111 | 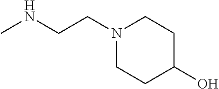 |
| XR112 | 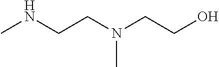 |
| XR113 | 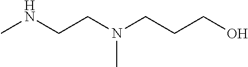 |
| XR114 | 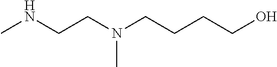 |
| XR115 | 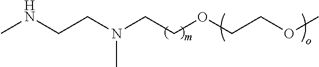 |
| XR116 | 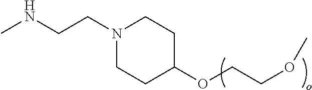 |
| XR117 | 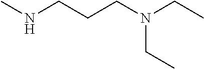 |
| XR118 | 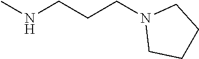 |
| XR119 | 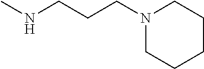 |
| XR120 | 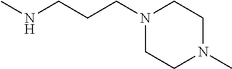 |
| XR121 | 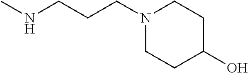 |
| XR122 | 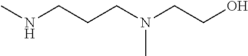 |
| XR123 | 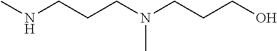 |
| XR124 | 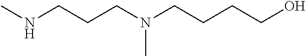 |
| XR125 | 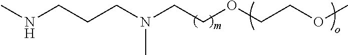 |
| XR126 | 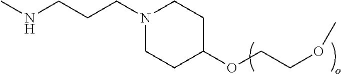 |
| XR127 | 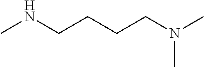 |
| XR128 | 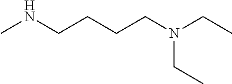 |
| XR129 | 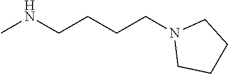 |
| XR130 | 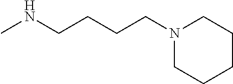 |
| XR131 | 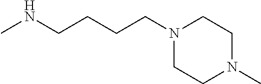 |
| XR132 | 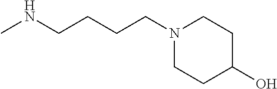 |
| XR133 | 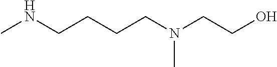 |
| XT134 | 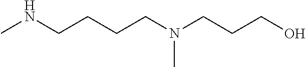 |
| XR135 | 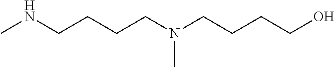 |
| XR136 | 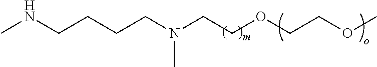 |
| XR137 | 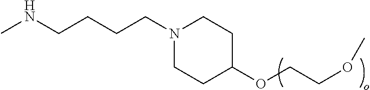 |
| XR138 | 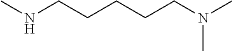 |
| XR139 | 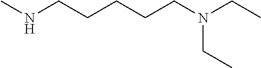 |
| XR140 | 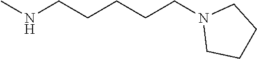 |
| XR141 | 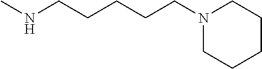 |
| XR142 | 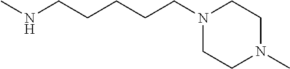 |
| XR143 | 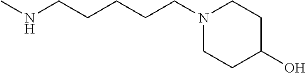 |
| XR144 | 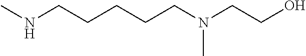 |
| XR145 | 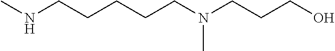 |
| XR146 | 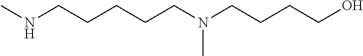 |
| XR147 | 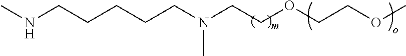 |
| XR148 | 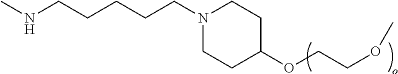 |
| XR149 | 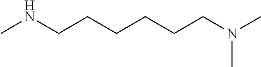 |
| XR150 | 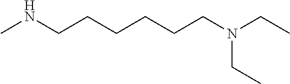 |
| XR151 | 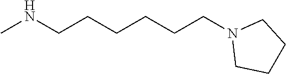 |
| XR152 | 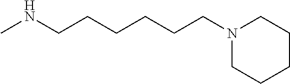 |
| XR153 | 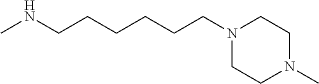 |
| XR154 | 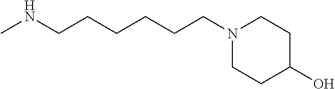 |
| XR155 | 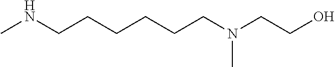 |
| XR156 | 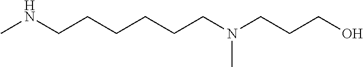 |
| XR157 | 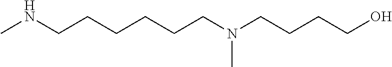 |
| XR158 | 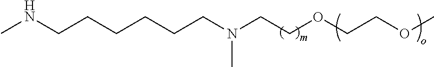 |
| XR159 | 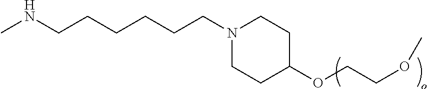 |

Reagents XR106-XR114, XR117-XR124, XR127-XR131, XR134, XR138-XR142, XR149-XR153, and XR156 are known in the art, as reported by the Chemical Abstract Society's SciFinder® with XR106-XR110, XR117-XR120, and XR127 being commercially available. XR132-XR133, XR135, XR143-XR146, XR154-XR154, and XR156 are prepared analogously to their shorter congeners. The polyethylene glycol-containing reagents are synthesized as disclosed in Example 4, as shown below.

The headgroup in Compound A-4, that is, X in Formula 1 is derived from 4-dimethylamino-butanoic acid. To obtain analogues of Compound A-4 with the disclosed alternative headgroups X and various values of n, the compounds of Table 4 may be used to substitute 4-dimethylamino-butanoic acid in the conversion of I-f.

TABLE 4
Alternative Headgroups of Compound A-4

| | |
|---|---|
| XR160 | 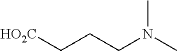 |
| XR161 | 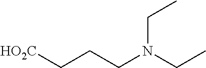 |
| XR162 | 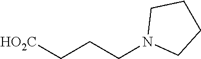 [SMILES: O=C(O)CCCN1CCCC1] |
| XR163 | 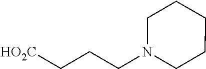 [SMILES: O=C(O)CCCN1CCCCC1] |
| XR164 | 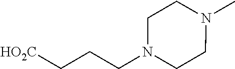 |
| XR165 | 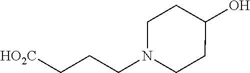 [SMILES: O=C(O)CCCN1CCC(O)CC1] |
| XR166 | 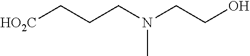 |
| XR167 | 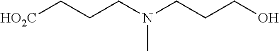 |
| XR168 | 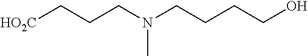 |
| XR169 | 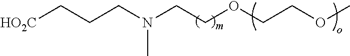 |
| XR170 | 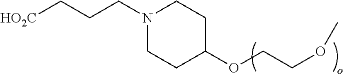 |
| XR171 | 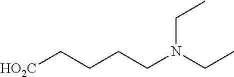 |
| XR172 | 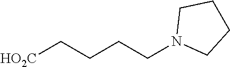 [SMILES: O=C(O)CCCCN1CCCC1] |
| XR173 | 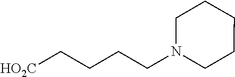 [SMILES: O=C(O)CCCCN1CCCCC1] |
| XR174 | 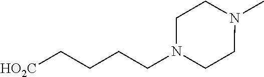 |
| XR175 | 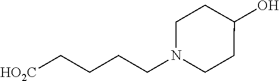 [SMILES: O=C(O)CCCCN1CCC(O)CC1] |
| XR176 | 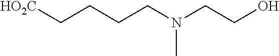 |
| XR177 | 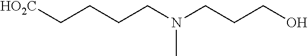 |
| XR178 | 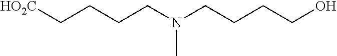 |
| XR179 | 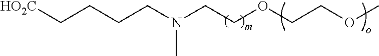 |
| XR180 | 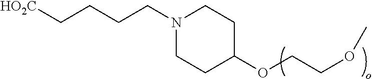 |
| XR181 | 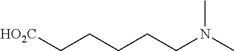 |
| XR182 | 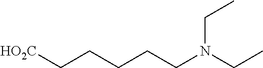 |
| XR183 | 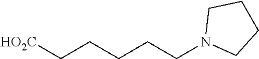 [SMILES: O=C(O)CCCCCN1CCCC1] |
| XR184 | 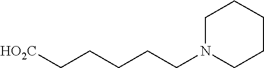 [SMILES: O=C(O)CCCCCN1CCCCC1] |
| XR185 | 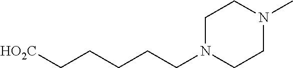 |
| XR186 | 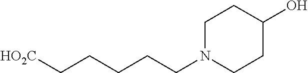 [SMILES: O=C(O)CCCCCN1CCC(O)CC1] |
| XR187 | 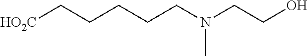 |
| XR188 | 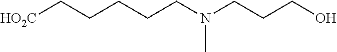 |
| XR189 | 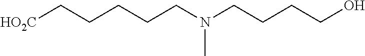 |
| XR190 | 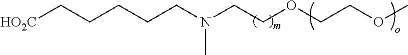 |
| XR191 | 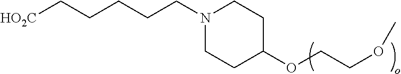 |
| XR192 | 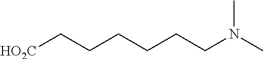 |
| XR193 | 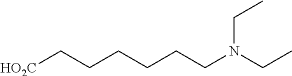 |
| XR194 | 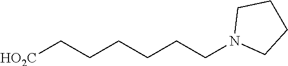 [SMILES: O=C(O)CCCCCCN1CCCC1] |
| XR195 | 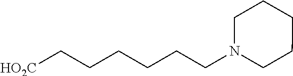 [SMILES: O=C(O)CCCCCCN1CCCCC1] |
| XR196 | 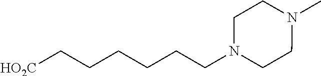 [SMILES: CN1CCN(CCCCCCC(=O)O)CC1] |
| XR197 | 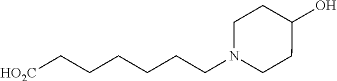 [SMILES: O=C(O)CCCCCCN1CCC(O)CC1] |
| XR198 | 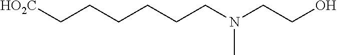 |
| XR199 | 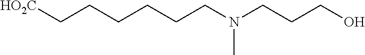 |
| XR200 | 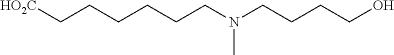 [SMILES: CN(CCCCO)CCCCCCC(=O)O] |
| XR201 | 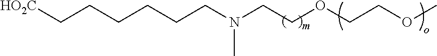 |
| XR202 | 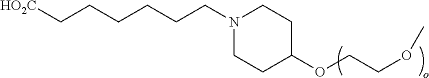 |
| XR203 | 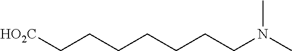 |
| XR204 | 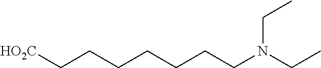 |
| XR205 | 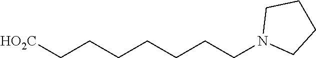 [SMILES: O=C(O)CCCCCCCN1CCCC1] |
| XR206 | 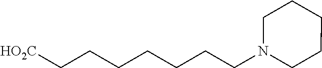 [SMILES: O=C(O)CCCCCCCN1CCCCC1] |
| XR207 | 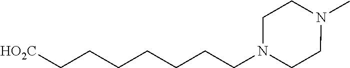 [SMILES: CN1CCN(CCCCCCCC(=O)O)CC1] |
| XR208 | 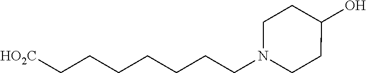 [SMILES: O=C(O)CCCCCCCN1CCC(O)CC1] |
| XR209 | 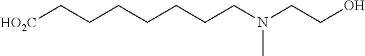 |
| XR210 | 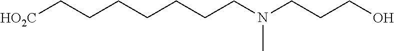 [SMILES: CN(CCCO)CCCCCCCC(=O)O] |
| XR211 | 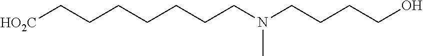 [SMILES: CN(CCCCO)CCCCCCCC(=O)O] |
| XR212 | 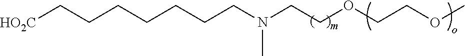 |
| XR213 | 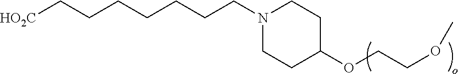 |

Reagents XR160-XR168, XR171-XR178, XR181-XR189, XR192-XR196, And XR203-XR206 are known in the art, as reported by the Chemical Abstract Society's SciFinder® with XR160, XR162-XR164, and XR181 being commercially available. XR196-XR199 and XR207-XR211 can be prepared analogously to their shorter congeners. The polyethylene glycol-containing reagents can be synthesized as described in Example 4, as shown below.

### Example 2: Synthesis of Compounds Having a Structure of Formula 2

The reaction of the sodium salt of BOC-blocked di-ethanolamine with 5-(2-bromoethyl)-2,2-dimethyl-1,3-dioxane II-a or 5-(2-bromoethyl)-2-phenyl-1,3-dioxane II-b leads to II-c and II-d, respectively. Different deprotection options are available, e.g., as exemplified by II-c and II-d, respectively. II-c can be deprotected with mild acid (PPTs, MeOH) to give diol II-e. Benzylidene acetal II-d can be deprotected with hydrogen and Pd/C to provide II-e. The coupling of II-e with decanoic acid (EDC-HCl, DMAP) provides II-f which upon deprotection of the BOC-protected amine upon exposure to CF₃CO₂H affords amine salt II-g. The reaction of amine salt II-g with carbonyl diimidazole leads to II-h, followed by the reaction of the acylimidazole II-h with methyl triflate to provide the intermediate that can be used for the synthesis of Compounds A-5 to A-7, acyl-imidazolium II-i (FIG. 2D). The reaction of II-i with 3-dimethylamino-1-propanol, in the presence of base, leads to carbamate Compound A-5, the reaction of II-i with N,N-dimethyl-1,3-propanediamine provides NH-urea Compound A-6, and the reaction of II-i with N,N,N′-trimethyl-1,3-propanediamine leads to Compound A-7 (FIG. 2E).

Amide Compound A-8 is obtained from the reaction of salt II-g with 4-dimethylamino-butanoic acid (EDC-HCl, DMAP, Et₃N) (FIG. 2F).

The headgroup in Compound A-5, that is, X in Formula 2 is derived from 3-dimethylamino-1-propanol. To obtain analogues of Compound A-5 with the disclosed alternative headgroups X and various values of n, the compounds of Table 1 (above) can be used to substitute 3-dimethylamino-1-propanol in the conversion of II-i.

The headgroup in Compound A-6, that is, X in Formula 2 is derived from N,N-dimethyl-1,3-propanediamine. To obtain analogues of Compound A-6 with the disclosed alternative headgroups X and various values of n, the compounds of Table 2 (above) can be used to substitute N,N-dimethyl-1,3-propanediamine in the conversion of II-i.

The headgroup in Compound A-7, that is, X in Formula 2 is derived from N,N,N′-trimethyl-1,3-propanediamine. To obtain analogues of Compound A-7 with the disclosed alternative headgroups X and various values of n, the compounds of Table 3 (above) can be used to substitute N,N,N′-trimethyl-1,3-propanediamine in the conversion of II-i.

The headgroup in Compound A-8, that is, X in Formula 2 is derived from 4-dimethylamino-butanoic acid. To obtain analogues of Compound A-8 with the disclosed alternative headgroups X and various values of n, the compounds of Table 4 (above) can be used to substitute 4-dimethylamino-butanoic acid in the conversion of II-g.

The polyethylene glycol-containing reagents are synthesized as disclosed in Example 4, below.

### Example 3: Synthesis of Compounds Having a Structure of Formula 3

The reaction of glutaric anhydride with dimethylamine gives 5-(dimethylamino)-5-oxopentanoic acid III-a. The coupling of III-a with diethanolamine in the presence of (4-(4,6-dimethoxy-1,3,5-triazin-2-yl)-4-methylmorpholinium chloride) gives N1,N1-bis(2-hydroxyethyl)-N5,N5-dimethyl-pentanediamide III-b, and reduction (LiAlH₄) provides diol III-c. Diol III-c coupling with acid I-d then affords Compound A-9 (FIG. 3C).

The reaction of the sodium salt (NaH, DMF) of III-c with either bromide II-a or II-b gives diamines III-d and III-e. Deprotection of III-d with PPTs in methanol or deprotection of III-e with hydrogen and palladium on carbon provides tetraol III-f. The coupling of tetraol III-f with decanoic acid (EDC-HCl, DMAP) leads to Compound A-10.

The headgroup in Compounds A-9 and A-10, that is, X in Formula 3 is derived from 5-(dimethylamino)-5-oxopentanoic acid (III-a), reacted with diethanolamine. To obtain analogues of Compounds A-9 and A-10 with the disclosed alternative headgroups X and various values of n, the carboxylic acids of Table 4 can be used to substitute 5-(dimethylamino)-5-oxopentanoic acid (III-a) according to the scheme:

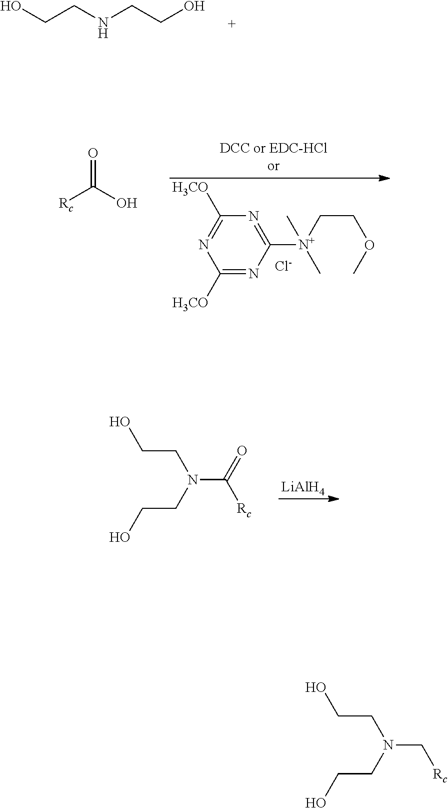

and the reactions are completed according to FIG. 3C-D, as appropriate.

The polyethylene glycol-containing reagents are synthesized as disclosed in Example 4, below.

### Example 4. Synthesis of Polyethylene Glycol-Containing Head Groups

The reaction of the sodium salt (NaH, DMF) of commercially available 1,1-dimethylethyl N-(3-hydroxypropyl)-N-methylcarbamate with the commercially available mesylate, 2-(2-(2-methoxyethoxy)ethoxy)ethyl methanesulfonate, provides V-5, which then affords amine V-6 after BOC removal (TFA) and neutralization. Reductive amination utilizing commercially available 1,1-dimethylethyl N-methyl-N-(3-oxopropyl)carbamate then yields BOC-protected amine V-7. The target PEG-containing head group piece V-8 (XR125 in Table 3) is then obtained after BOC removal (TFA) and amine neutralization. See FIG. 4A.

Similarly, the reaction of the sodium salt (NaH, DMF) of commercially available BOC-blocked 4-hydroxypiperidine with commercially available 2-(2-(2-methoxyethoxy)ethoxy)ethyl methanesulfonate provides V-1, which then affords amine V-2 after BOC removal (TFA) and neutralization. Reductive amination utilizing commercially available 1,1-dimethylethyl N-methyl-N-(3-oxopropyl)carbamate then yields BOC-protected amine V-3. The target PEG-containing head group piece V-4 (XR126 in Table 3) is then obtained after BOC removal (TFA) and amine neutralization. See FIG. 4B.

Shorter chain PEG-containing head group entities can be obtained by substituting the known/commercially available shorter chain mesylates V-9 (known) and V-10 (commercially available) for the 2-(2-(2-methoxyethoxy)ethoxy)ethyl methanesulfonate utilized in the schemes above.

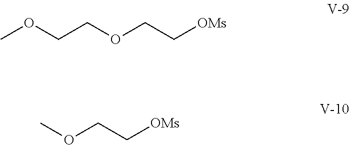

To synthesize variants of V-4 with different values of n or alternate Y definitions, appropriate analogues of the 1,1-dimethylethyl N-methyl-N-(3-oxopropyl)carbamate are used to create the desired PEG-containing head group piece. For variants of V-8 one uses appropriate analogues of the 1,1-dimethylethyl N-(3-hydroxypropyl)-N-methylcarbamate to bring in different values of m and analogues of the 1,1-dimethylethyl N-methyl-N-(3-oxopropyl)carbamate to bring in different values of n or definitions of Y to create the desired PEG-containing head group piece.

### Example 5. Synthesis of 1,1-Dimethylethyl 4-hydroxy-3-(hydroxymethyl)-2-butenoate (I-a)

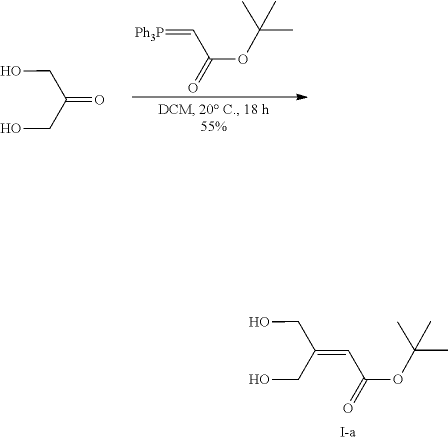

To a solution of dihydroxyacetone (150.0 g, 1.67 mol) in anhydrous dichloromethane (3.0 L), under nitrogen) was added tert-butoxycarbonylmethylene-triphenylphosphorane (627 g, 1.67 mol) in portions over 1 hour. The mixture was stirred for 18 hours at room temperature, then silica gel (750 g, type: ZCX-2, 100-200 mesh) was added to the solution and the solvent was removed in vacuo to afford crude 1 impregnated on silica gel. The dry silica gel was placed onto a gravity column of silica gel (3700 g, type: ZCX-2, 100-200 mesh, packed with petroleum ether), and the resulting column was eluted with a gradient of petroleum ether:ethyl acetate (100:0 to 50:50). Compound I-a eluted with petroleum ether:ethyl acetate 50:50 and the fractions of I-a were concentrated in vacuo to provide 1 (235.0 g) containing Ph₃PO (purity 73.8% by HNMR, 55% yield of I-a).

¹H NMR (400 MHz, DMSO-d6, ppm): δ 5.77 (m, 1H), 5.00 (t, J=5.6 Hz, 1H), 4.80 (t, J=5.7 Hz, 1H), 4.49 (dd, J=5.8, 1.5 Hz, 2H), 4.17 (dd, J=5.6, 2.0 Hz, 2H), 1.42 (s, 9H); LCMS (+ mode): Calcd. for C₉H₁₆O₄+H⁺: 189.11, Found: 189.10.

### Example 6. Synthesis of tert-Butyl 4-hydroxy-3-(hydroxymethyl)butanoate (I-b)

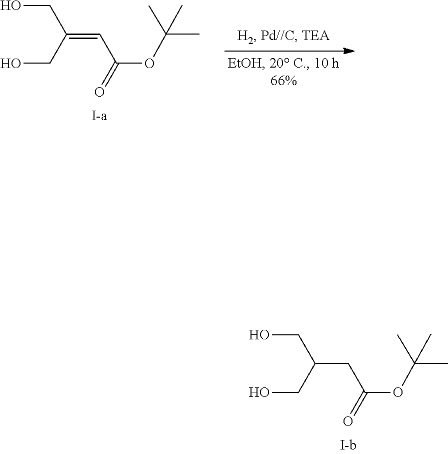

To a solution of I-a (100.00 g, 73.8% pure, 0.53 mol) in anhydrous EtOH (1.0 L), in a 2.0 L round bottom flask under nitrogen, was added Et₃N (8.10 g, 0.080 mol) followed by 10% Pd/C (20.0 g). The mixture was placed under a hydrogen balloon for 16 hours at room temperature. HPLC analysis indicated that the hydrogenation was not complete and the hydrogen balloon was refilled and the reaction was continued for an additional 16 hours. The mixture was filtered through a pad of Celite®, the filter cake was rinsed with absolute EtOH (200 mL) and the combined filtrates were concentrated in vacuo to afford I-b (198.0 g) containing P₃PO (purity 67.5% by HNMR, 66% yield of I-b) as a yellow oil.

¹H NMR (400 MHz, DMSO-D6, ppm): d 4.47 (t, J=5.10 Hz, 2H), 3.44-3.29 (5H), 2.17 (d, J=7.00 Hz, 2H), 1.93 (m, 1H), 1.39 (s, 9H).

### Example 7. Synthesis of 2-(2-(tert-Butoxy)-2-oxoethyl)propane-1,3-diyl dinonanoate (I-c2)

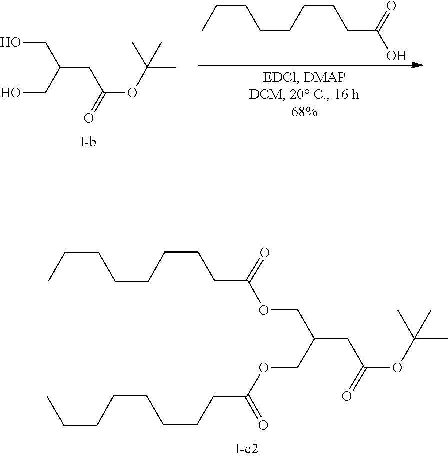

To a solution of I-b (120.00 g, 67.5% pure, 0.63 mol) in CH₂Cl₂ (1.2 L), in a 4 L flask under nitrogen, was added in order nonanoic acid (199.60 g, 1.26 mol, 2.0 eq.) and DMAP (77.10 g, 0.63 mol). The mixture was stirred for 10 minutes at room temperature, then EDCI (266.8 g, 1.39 mol, 2.20 eq) was added in one portion. The mixture was allowed to stir for 16 h at room temperature then the solvent was removed in vacuo to provide crude I-c2 as a sticky, yellow oil. Crude I-c2 was dissolved in heptane/methyl tert-butyl ether (MTBE) (9:1, 2.0 L) and the solution was washed with 10% aq. citric acid (2×1.2 L). The organic phase was washed with brine (2.0 L), MeOH:H₂O (5:1, 3×1.20 L), and then dried over anhydrous Na₂SO₄. The solids were removed by filtration and silica gel (400 g, type: ZCX-2, 100-200 mesh) was added to the solution. The solvent was removed in vacuo to give crude I-c2 impregnated on silica gel. The dry silica gel was placed onto a gravity column of silica gel (4000 g, type: ZCX-2, 100-200 mesh, packed with petroleum ether), and the resulting column was eluted with a gradient of petroleum ether:THF (100:0 to 95:5). Compound I-c2 eluted with petroleum ether:THF 98:2 and the fractions of I-c2 were concentrated in vacuo to provide I-c2 as a colorless oil (208.0 g, purity 97.2% by HPLC, 68% yield).

¹H NMR (400 MHz, CDCl₃, ppm): d 4.11 (m, 4H), 2.52 (m, 1H), 2.31 (t, J=7.40 Hz, 4H), 1.62 (m, 4H), 1.46 (s, 9H), 1.35-1.27 (20H), 0.87 (m, 6H).

When used in the synthesis of a lipid accord to Formula 1 or 3 according to the synthetic schemes disclosed herein, I-c2 leads to a lipid in which R is straight-chain C₈, whereas I-c leads to a lipid in which R is straight-chain C₉.

### Example 8. Synthesis of 4-(Nonanolyoxy)-3-((nonanoyloxy)methyl)butanoic acid (I-d2)

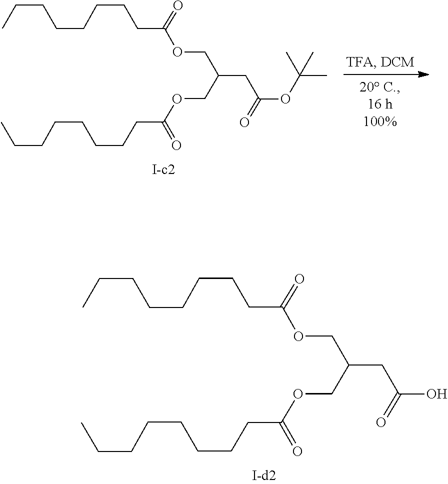

To a solution of I-c2 (110.0 g, 0.234 mol) in CH₂Cl₂ (1.0 L), at room temperature under nitrogen, was added trifluoroacetic acid (TFA) (330 mL, 4.31 mol) over 30 minutes. The resulting solution was stirred for 16 h at room temperature, then was concentrated in vacuo to provide crude I-d2 as a yellow oil. Crude I-d2 was dissolved in CH₂Cl₂ (1.10 L) and the solution was washed with water (2×0.50 L). The combined aq. phases were extracted with CH₂Cl₂ (0.5 L), and the combined organic layers were dried over anhydrous MgSO₄. Filtration and concentration in vacuo gave I-d2 as a pale brown oil (96.0 g, 0.232 mol, 99%, 98.5% purity by HPLC).

¹H NMR (400 MHz, CDCl₃, ppm): d 4.13 (m, 4H), 2.58 (m, 1H), 2.49 (d, II=6.90 Hz, 2H), 2.33 (t, J=7.60 Hz, 4H), 1.63 (m, 4H), 1.36-1.22 (20H), 0.89 (t, J=6.60 Hz, 6H); LCMS (+ mode): Calcd. for C₂₃H₄₂O₆+H⁺: 415.31. Found: 415.30.

When used in the synthesis of a lipid accord to Formula 1 or 3 according to the synthetic schemes disclosed herein, I-d2 leads to a lipid in which R is straight-chain C₈, whereas 1-d leads to a lipid in which R is straight-chain C₉.

### Example 9. Synthesis of (((((tert-butoxycarbonyl)azanediyl)bis(ethane-2,1-diyl))bis(oxy))bis(2-oxoethane-2,1-diyl))bis(propane-2,1,3-triyl) tetranonanoate (I-e2)

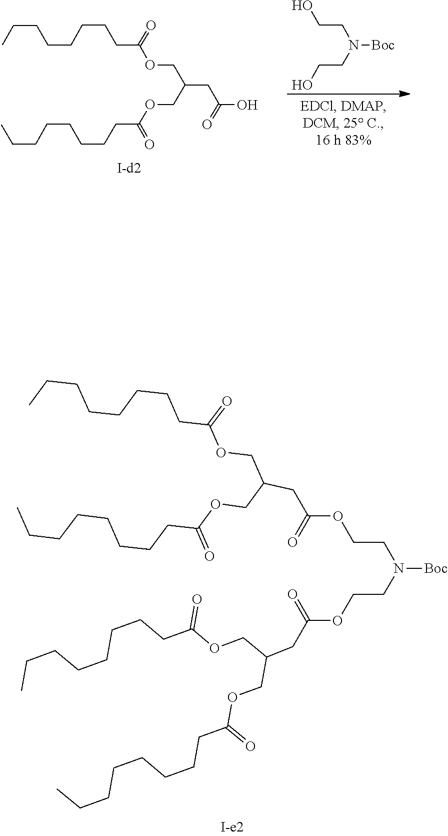 [SMILES: CCCCCCCCC(=O)OCC(COC(=O)CCCCCCCC)CC(=O)O ; CC(C)(C)OC(=O)N(CCO)CCO ; CCCCCCCCC(=O)OCC(COC(=O)CCCCCCCC)CC(=O)OCCN(CCOC(=O)CC(COC(=O)CCCCCCCC)COC(=O)CCCCCCCC)C(=O)OC(C)(C)C]

To a solution of I-d2 (142.8 g, 0.344 mol) in CH₂Cl₂ (1.0 L), at room temperature under nitrogen was added in order tert-butyl bis(2-hydroxyethyl)carbamate (33.6 g, 0.164 mol) and DMAP (20.0 g, 0.164 mol). The mixture was stirred for 10 minutes, then EDCI (78.7 g, 0.410 mol) was added over a period of 10 minutes, and the resulting solution was stirred for 16 hours at room temperature. The reaction mixture was washed with water (2×1.2 L), the aq. phase was extracted with CH₂Cl₂ (2×1.2 L) and the combined organic phases were dried over anhydrous MgSO₄. After filtration to remove the solids, the solution was concentrated in vacuo to ca. 1.5 L volume and silica gel (350 g, type: ZCX-2, 100-200 mesh) was added and the mixture was concentrated in vacuo to dryness. The dry silica gel was placed onto a gravity column of silica gel (2100 g, type: ZCX-2, 100-200 mesh, packed with heptane), and the resulting column was eluted with a gradient of heptane:THF (100:0 to 90:10). Compound I-e2 eluted with heptane:THF 95:5 and the fractions of I-e2 were concentrated in vacuo to provide I-e2 as a yellow oil (143.5 g, purity 96.3% by HPLC, 83% yield).

¹H NMR (400 MHz, CDCl₃, ppm): d 4.21 (m, 4H), 4.11 (m, 8H), 3.49 (m, 4H), 2.58 (m, 2H), 2.43 (d, J=6.90 Hz, 4H), 2.31 (t, J=7.60 Hz, 8H), 1.66-1.59 (8H), 1.48 (s, 9H), 1.20-1.39 (40H), 0.89 (t, J=6.60 Hz, 12H).

When used in the synthesis of a lipid accord to Formula 1 or 3 according to the synthetic schemes disclosed herein, I-e2 leads to a lipid in which R is straight-chain C₈, whereas I-e leads to a lipid in which R is straight-chain C₉.

### Example 10. Synthesis of bis(2-((4-(Nonanoyloxy)-3-((nonanoyloxy)methyl)butanoyl)oxy)ethyl)amine trifluoroacetic acid salt (I-f2)

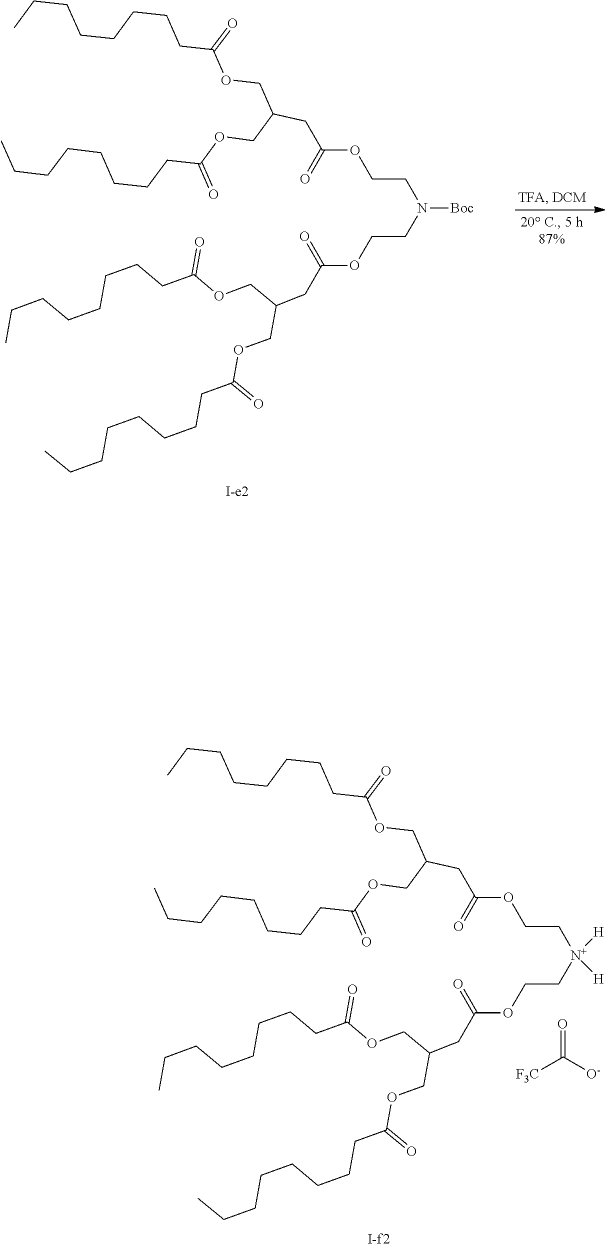 [SMILES: CCCCCCCCC(=O)OCC(COC(=O)CCCCCCCC)CC(=O)OCCN(CCOC(=O)CC(COC(=O)CCCCCCCC)COC(=O)CCCCCCCC)C(=O)OC(C)(C)C ; CCCCCCCCC(=O)OCC(COC(=O)CCCCCCCC)CC(=O)OCC[NH2+]CCOC(=O)CC(COC(=O)CCCCCCCC)COC(=O)CCCCCCCC ; O=C([O-])C(F)(F)F]

To a solution of I-e2 (143.0 g, 0.137 mol) in CH₂Cl₂ (850 mL), at room temperature under nitrogen, was added TFA (286 mL, 3.74 mol) over a period of 30 minutes. After the addition was complete the mixture was allowed to stir for 5 hours, then the solution was cast into 10% aq. K₂HPO₄ (1.40 L) and water (0.70 L). The organic phase was dried (MgSO₄), filtered and concentrated in vacuo to give the ammonium salt 6 as a yellow oil (140.5 g, 89% purity by HPLC, 87% yield).

¹H NMR (400 MHz, CDCl₃, ppm): d 4.79 (m, 4H), 4.20 (m, 4H), 4.10 (m, 4H), 3.46 (m, 4H), 2.55 (m, 2H), 2.42 (d, J=7.10 Hz, 4H), 2.34 (t, J=7.60 Hz, 8H), 1.62 (m, 8H), 1.21-1.38 (40H), 0.90 (m, 12H); LCMS (+ mode): Calcd. for C₅₀H₉₂NO₁₂: 898.66. Found: 899.0.

When used in the synthesis of a lipid accord to Formula 1 or 3 according to the synthetic schemes disclosed herein, I-e2 leads to a lipid in which R is straight-chain C₈, whereas I-e leads to a lipid in which R is straight-chain C₉.

### Example 11. Synthesis of (((((1H-imidazole-1-carbonyl)azanediyl)bis(ethane-2,1-diyl))bis(oxy))bis(2-oxoethane-2,1-diyl))bis(propane-2,1,3-triyl) tetranonanoate (I-g2)

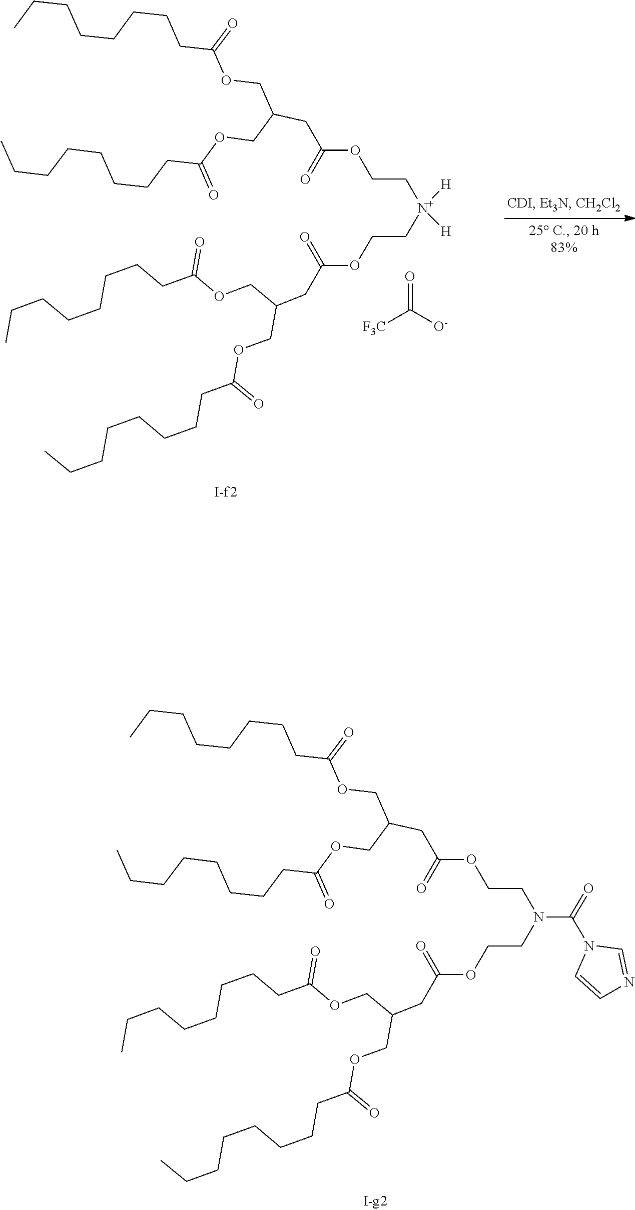 [SMILES: CCCCCCCCC(=O)OCC(COC(=O)CCCCCCCC)CC(=O)OCC[NH2+]CCOC(=O)CC(COC(=O)CCCCCCCC)COC(=O)CCCCCCCC ; O=C([O-])C(F)(F)F ; CCCCCCCCC(=O)OCC(COC(=O)CCCCCCCC)CC(=O)OCCN(CCOC(=O)CC(COC(=O)CCCCCCCC)COC(=O)CCCCCCCC)C(=O)n1ccnc1]

To a solution of I-f2 (130.0 g, 0.123 mol), in CH₂Cl₂ (1.30 L) at room temperature under nitrogen, was added in order: carbonyl diimidazole (CDI) (79.8 g, 0.493 mol) and Et₃N (25.2 g, 0.246 mol). The resulting solution was stirred for 3 h at room temperature. HPLC analysis indicated that the reaction was not complete, and additional CDI (39.9 g, 0.246 mol) and Et₃N (14.6 g, 0.123 mol) were added. The solution was allowed to stir for 14 hours at room temperature, then the mixture was cast into aq. HCl (0.8M, 1.30 L). The organic phase was separated, and the aq. phase was extracted with CH₂Cl₂ (1.30 L). The combined organic phases were concentrated in vacuo to furnish crude I-g2 as a yellow oil which was dissolved in heptane (1.30 L). The heptane solution was washed with MeOH—H₂O (5:1, 2×0.65 L) and brine (0.65 L). The heptane solution was dried (MgSO₄), the solids were removed by filtration and the filtrate was concentrated in vacuo to give 7 (122.0 g, purity by HPLC 83.3%, 83% yield) as a viscous, yellow oil.

¹H NMR (400 MHz, CDCl₃, ppm): d 7.99 (s, 1H), 7.31 (s, 1H), 7.14 (s, 1H), 4.32 (m, 4H), 4.12 (m, 8H), 3.77 (m, 4H), 2.55 (m, 2H), 2.42 (d, J=6.90 Hz, 4H), 2.31 (m, 8H), 1.62 (m, 8H), 1.21-1.33 (40H), 0.90 (m, 12H); LCMS (+ mode): Calcd. for C₅₄H₉₃N₃O₁₃: 991.67. Found: 993.0.

### Example 12. Synthesis of 1-(bis(2-((4-(nonanoyloxy)-3-((nonanoyloxy)methyl)butanoyl)oxy)ethyl)carbamoyl)-3-methyl-1H-imidazol-3-ium trifluoromethanesulfonate (I-h2)

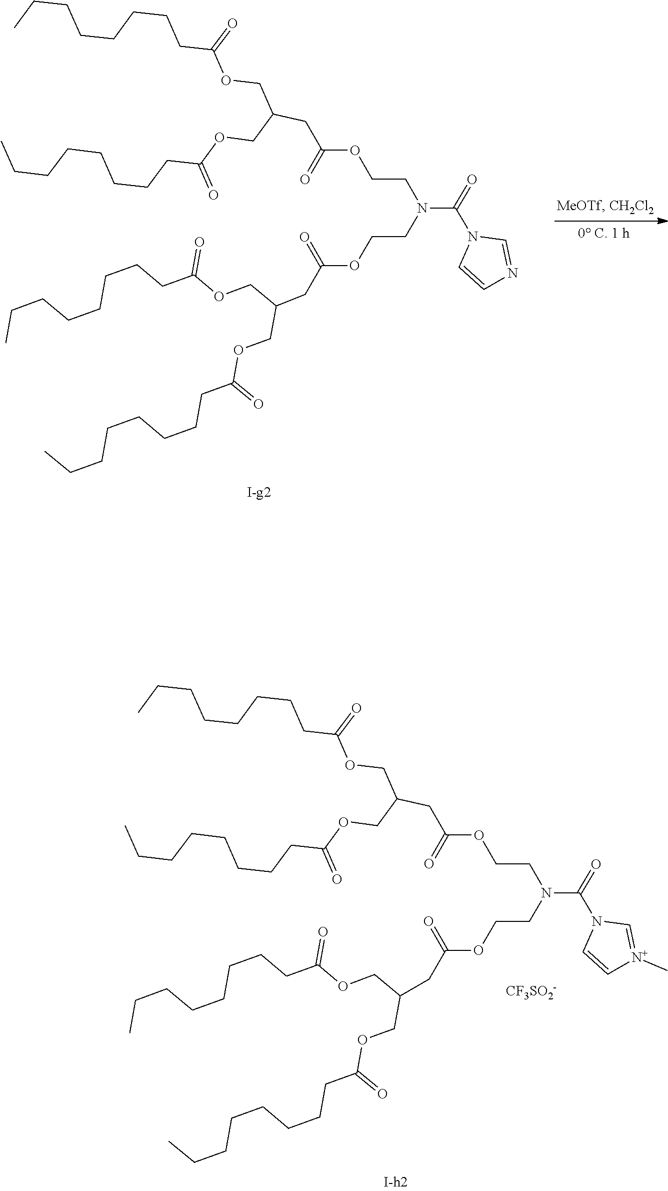

Acyl-imidazole I-g2 (18.0 g, 18.2 mmol) was dissolved in CH₂Cl₂ (270 mL), under nitrogen, and was cooled in an ice water bath. To this cooled solution of I-g2 was added methyl trifluoromethanesulfonate (MeOTf) (3.30 g, 20.0 mmol) over a period of 15 minutes. The resulting solution was allowed to stir for 1 hour at 0° C., then was carried on to the target lipids (vide infra). HPLC and LCMS indicated complete consumption of I-g2.

### Example 13. Synthesis of Compound A-11

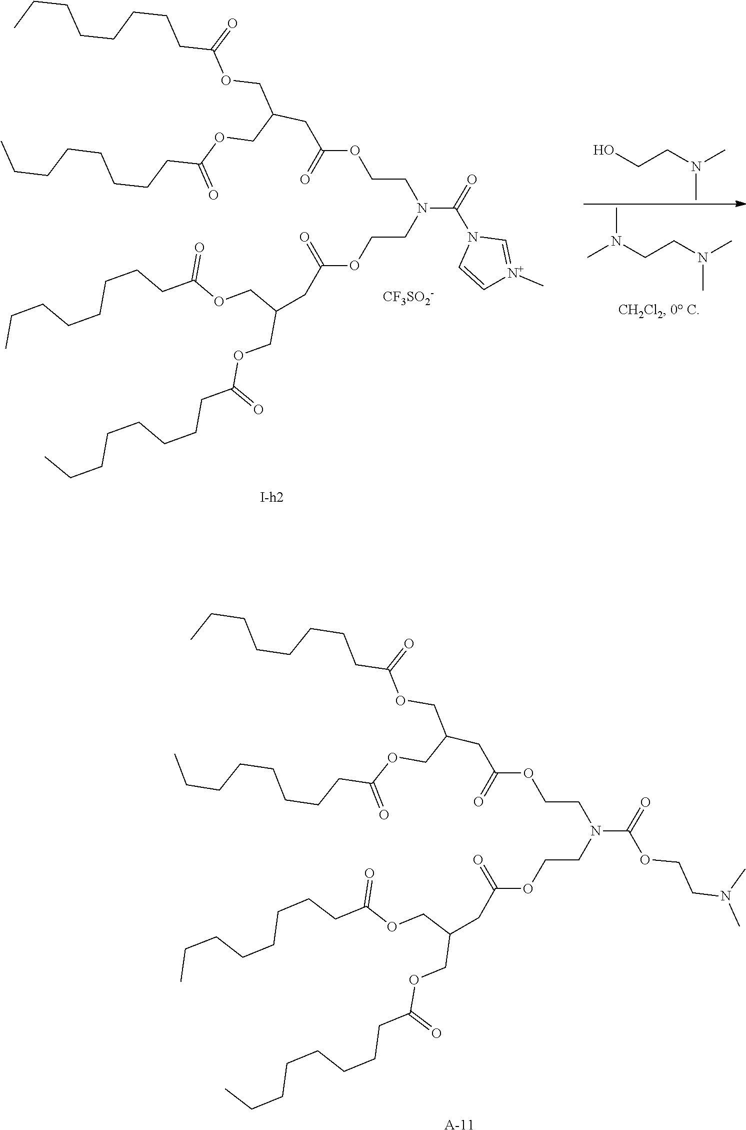

To a solution of I-h2, prepared as described above from I-g2 (20.2 mmol), cooled in an ice-water bath under nitrogen, was added in order tetramethylethylene diamine (11.70 g, 100.8 mmol) and 2-dimethylamino-ethanol (3.60 g, 40.3 mmol). The mixture was stirred for 1 hour at 0° C., then was warmed to room temperature and stirred for 18 hours. The solution was concentrated in vacuo and the residue was dissolved in ethyl acetate (300 mL). The solution was washed with 10% aq. citric acid (2×300 mL), the organic phase was separated, and the aq. phase was extracted with ethyl acetate (300 mL). The combined organic phases were washed with 5% aq. NaHCO₃ (300 mL), brine (300 mL) and dried (MgSO₄). The solids were removed by filtration and silica gel (40 g, type: ZCX-2, 100-200 mesh) was added to the solution, and the mixture was concentrated in vacuo to dryness. The dry silica gel was placed onto a gravity column of silica gel (200 g, type: ZCX-2, 100-200 mesh, packed with CH₂Cl₂), and the resulting column was eluted with a gradient of CH₂Cl₂:MeOH (100:0 to 90:10). Compound A-11 eluted with CH₂Cl₂:MeOH 95:5 and the fractions of Compound A-11 were concentrated in vacuo to provide Compound A-11 as a yellow oil (12.0 g, HPLC purity 88%). Compound A-11 was further purified by reverse phase flash chromatography (WelFlash XSelect CSH Prep C18, 5 mm OBD, Regular 30×150 mm column; Solvents: A: 0.1% formic acid in water, B: acetonitrile, gradient 50-80%, 20 minutes, flow 55 mL/min). Fractions containing Compound A-11 were pooled, and concentrated in vacuo and the residue was dissolved in heptane (150 mL). The heptane solution was washed with MeOH/water (75:25, 100 mL) and brine (100 mL). The organic phase was dried (Na₂SO₄), the solids were removed by filtration, and the filtrate was concentrated in vacuo to afford Compound A-11 (10.15 g, 97.8% purity by HPLC, 49% yield) as a yellow oil.

¹H NMR (400 MHz, CDCl₃, ppm): d 4.20 (m, 6H), 4.09 (m, 8H), 3.54 (m, 4H), 2.55 (m, 4H), 2.41 (d, J=6.90 Hz, 4H), 2.25-2.32 (14H), 1.61 (m, 8H), 1.20-1.34 (40H), 0.88 (t, J=6.70 Hz, 12H); LCMS (+ mode): Calcd. for C₅₅H₁₀₀N₂O₁₄+H⁺: 1013.72. Found: 1013.80 [M+H⁺].

### Example 14. Synthesis of Compound A-12

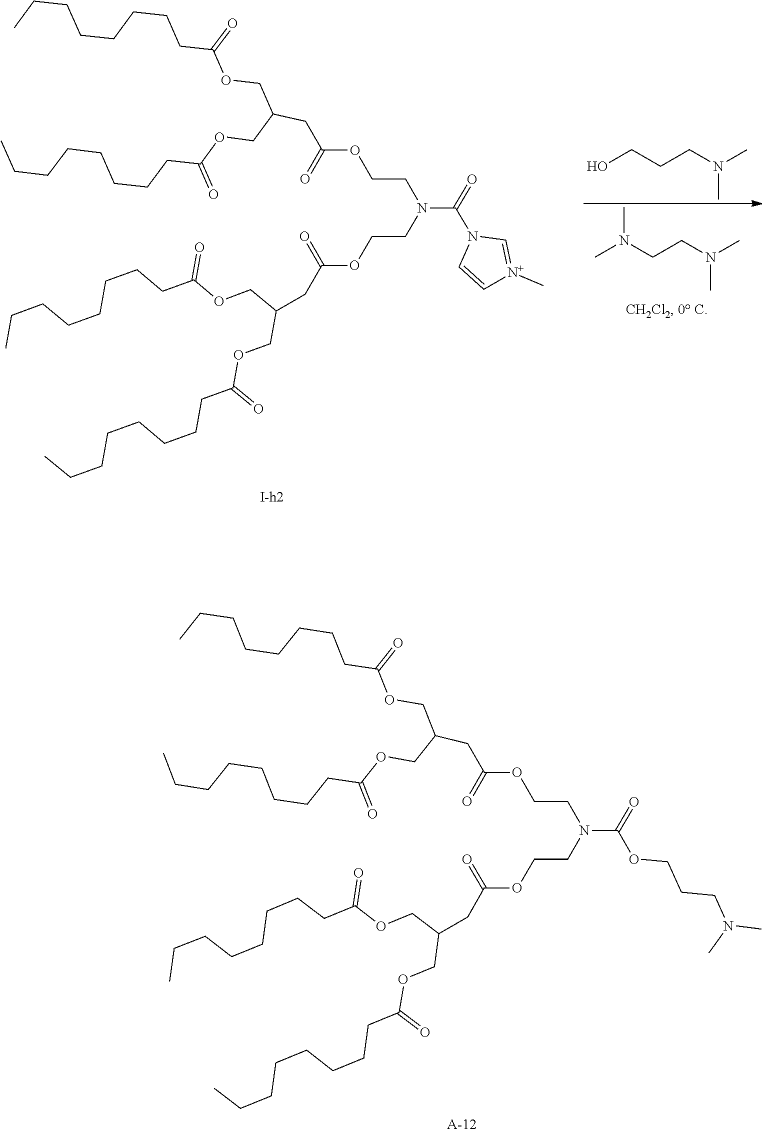

To a solution of I-h2, prepared as described above from I-g2 (18.2 mmol), cooled in an ice-water bath under nitrogen, was added in order tetramethylethylene diamine (6.30 g, 54.4 mmol) and 3-dimethylamino-propanol (2.20 g, 21.8 mmol). The mixture was stirred for 1 hour at 0° C., then warmed to room temperature and stirred for 18 hours. The solution was concentrated in vacuo and the residue was dissolved in ethyl acetate (300 mL). The solution was washed with 10% aq. citric acid (2×300 mL), the organic phase was separated, and the aq. phase was extracted with ethyl acetate (300 mL). The combined organic phases were washed with 5% aq. NaHCO₃ (300 mL), brine (300 mL) and dried (MgSO₄). The solids were removed by filtration and silica gel (36 g, type: ZCX-2, 100-200 mesh) was added to the solution, and the mixture was concentrated in vacuo to dryness. The dry silica gel was placed onto a gravity column of silica gel (180 g, type: ZCX-2, 100-200 mesh, packed with CH₂Cl₂), and the resulting column was eluted with a gradient of CH₂Cl₂:MeOH (100:0 to 90:10). Compound A-12 eluted with CH₂Cl₂:MeOH 95:5 and the fractions of Compound A-12 were concentrated in vacuo to provide Compound A-12 as a yellow oil (11.2 g, HPLC purity 85%). Compound A-12 was further purified by reverse phase flash chromatography (WelFlash XSelect CSH Prep C18, 5 mm OBD, Regular 30×150 mm column; Solvents: A: 0.1% formic acid in water, B: acetonitrile, gradient 50-80%, 20 minutes, flow 55 mL/min). Fractions containing Compound A-12 were pooled and concentrated in vacuo and the residue was dissolved in heptane (150 mL). The heptane solution was washed with 5% aq. Na₂CO₃ (100 mL), MeOH/water (75:25, 100 mL) and brine (100 mL). The organic phase was dried (Na₂SO₄), the solids were removed by filtration, and the filtrate was concentrated in vacuo to afford Compound A-12 (10.22 g, 96.8% purity by HPLC, 48% yield) as a yellow oil.

¹H NMR (400 MHz, CDCl₃, ppm): d 4.06-4.23 (14H), 3.53 (m, 4H), 2.55 (m, 2H), 2.41 (d, J=6.90 Hz, 6H), 2.27-2.32 (14H), 1.85 (m, 2H), 1.57-1.62 (8H), 1.20-1.35 (40H), 0.88 (t, J=6.80 Hz, 12H); LCMS (+ mode): Calcd. for C₅₆H₁₀₂N₂O₁₄+H⁺: 1027.74. Found: 1027.90 [M+H⁺].

### Example 15. Synthesis of Compound A-13

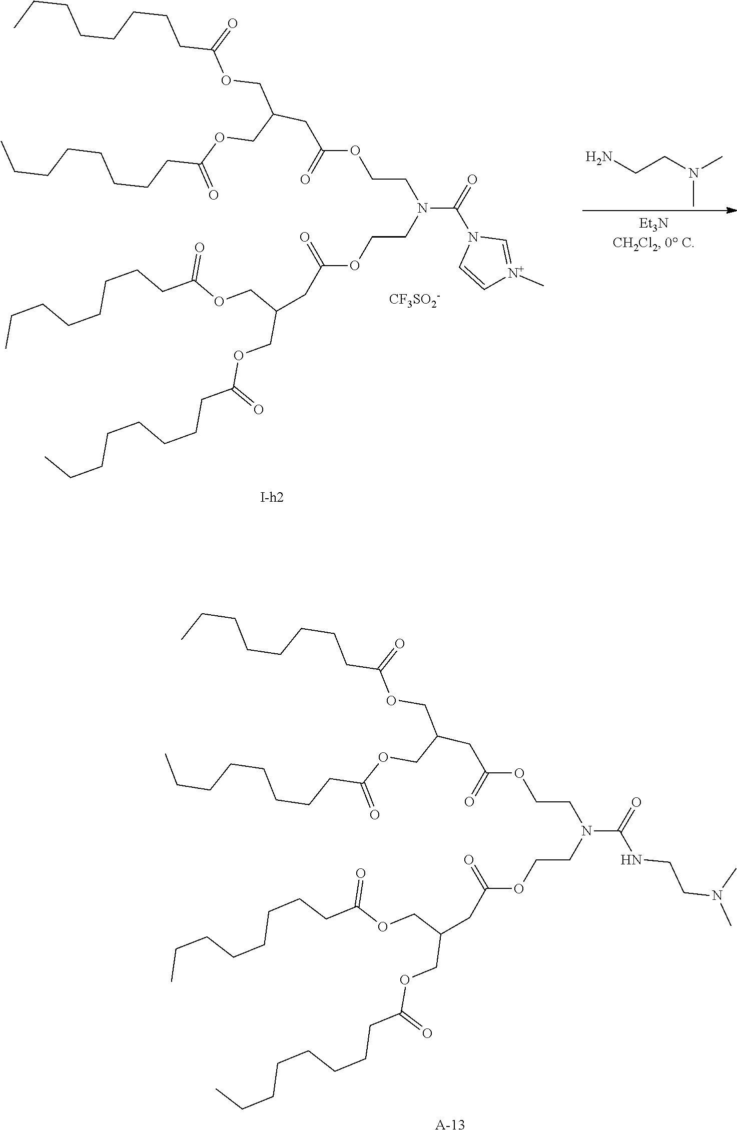

To a solution of I-h2, prepared as described above from I-g2 (22.0 mmol), cooled in an ice-water bath under nitrogen, was added in order triethylamine (6.72 g, 66.0 mmol) and 2-dimethylamino-ethylamine (2.34 g, 26.0 mmol). The mixture was stirred for 1 hour at 0° C., then was warmed to room temperature and stirred for 18 hours. The solution was concentrated in vacuo and the residue was dissolved in ethyl acetate (600 mL). The solution was washed with 5% aq. Na₂CO₃ (2×300 mL), brine (300 mL) and dried (MgSO₄). The solids were removed by filtration and silica gel 40 g, type: ZCX-2, 100-200 mesh) was added to the solution, and the mixture was concentrated in vacuo to dryness. The dry silica gel was placed onto a gravity column of silica gel (250 g, type: ZCX-2, 100-200 mesh, packed with CH₂Cl₂), and the resulting column was eluted with a gradient of CH₂Cl₂:MeOH (100:0 to 90:10). Compound A-13 eluted with CH₂Cl₂:MeOH 97:3 and the fractions of Compound A-13 were concentrated in vacuo to provide Compound A-13 as a yellow oil (18.0 g, HPLC purity 83%). Compound A-13 was further purified by reverse phase flash chromatography (WelFlash XSelect CSH Prep C18, 5 mm OBD, Regular 30×150 mm column; Solvents: A: 0.1% formic acid in water, B: acetonitrile, gradient 50-80%, 20 minutes, flow 55 mL/min). Fractions containing Compound A-13 were pooled and concentrated in vacuo and the residue was dissolved in heptane (150 mL). The heptane solution was washed with satd. aq. NaHO₃ (200 mL), MeOH/water (80:20, 2×200 mL) and brine (200 mL). The organic phase was dried (Na₂SO₄), the solids were removed by filtration, and the filtrate was concentrated in vacuo to afford Compound A-13 (10.58 g, 95.5% purity by HPLC, 47% yield) as a yellow oil.

¹H NMR (300 MHz, CDCl₃, ppm): d 5.50 (brs, 1H), 4.22 (t, J=6.00 Hz, 4H), 4.03-4.20 (8H), 3.52 (m, 4H), 3.30 (m, 2H), 2.56 (m, 2H), 2.32-2.46 (6H), 2.20-2.32 (14H), 2.50-2.65 (8H), 1.15-1.32 (40H), 0.88 (m, 12H); LCMS (+ mode): Calcd. for C₅₄H₁₀₁N₃O₁₃+H⁺: 1012.74. Found: 1012.80 [M+H⁺].

### Example 16. Synthesis of Compound A-14

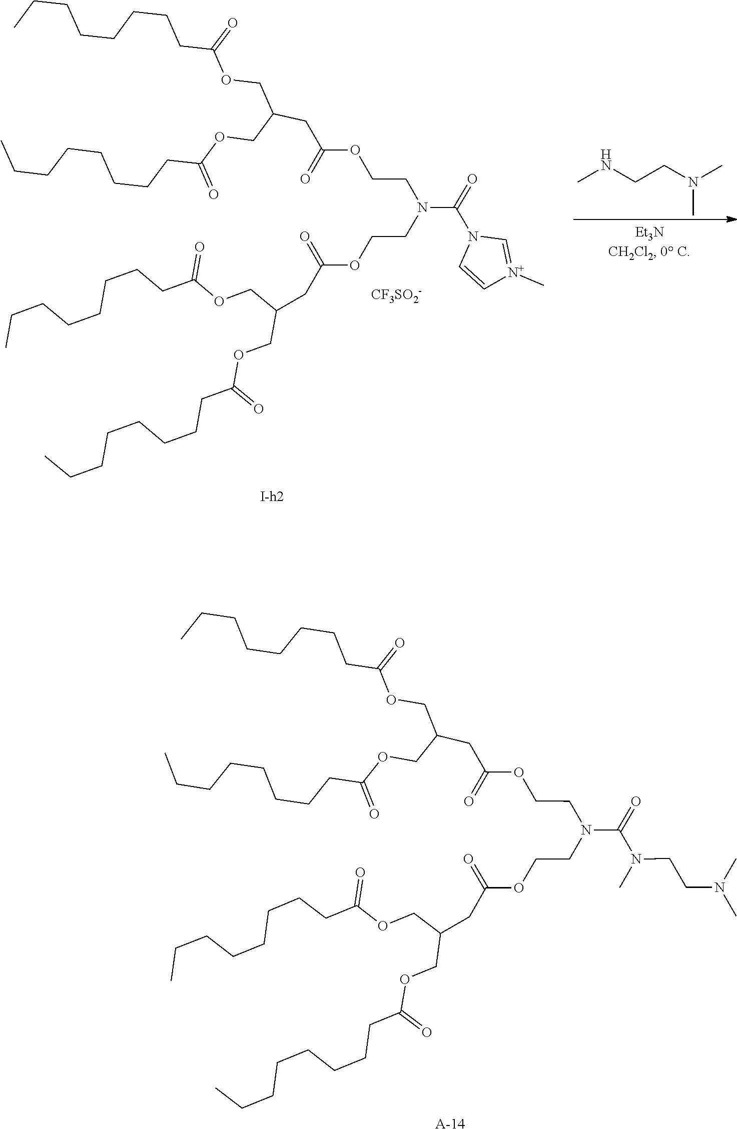

To a solution of I-h2, prepared as described above from I-g2 (22.0 mmol), cooled in an ice-water bath under nitrogen, was added in order triethylamine (6.72 g, 66.0 mmol) and N,N,N′-trimethylethylenediamine (2.71 g, 26.0 mmol). The mixture was stirred for 1 hour at 0° C., then was warmed to room temperature and stirred for 18 hours. The solution was concentrated in vacuo and the residue was dissolved in ethyl acetate (600 mL). The solution was washed with 10% aq. citric acid (2×300 mL), 5% aq. Na₂CO₃ (2×300 mL), brine (300 mL), and dried (MgSO₄). The solids were removed by filtration and silica gel (40 g, type: ZCX-2, 100-200 mesh) was added to the solution, and the mixture was concentrated in vacuo to dryness. The dry silica gel was placed onto a gravity column of silica gel (250 g, type: ZCX-2, 100-200 mesh, packed with CH₂Cl₂), and the resulting column was eluted with a gradient of CH₂Cl₂:MeOH (100:0 to 90:10). Compound A-14 eluted with CH₂Cl₂:MeOH 97:3 and the fractions of Compound A-14 were concentrated in vacuo to provide Compound A-14 as a yellow oil (18.0 g, HPLC purity 88%). Compound A-14 was further purified by reverse phase flash chromatography (WelFlash XSelect CSH Prep C18, 5 mm OBD, Regular 30×150 mm column; Solvents: A: 0.1% formic acid in water, B: acetonitrile, gradient 50-80%, 20 minutes, flow 55 mL/min). Fractions containing Compound A-14 were pooled and concentrated in vacuo and the residue was dissolved in heptane (500 mL). The heptane solution was washed with satd. aq. NaHO₃ (500 mL), MeOH/water (80:20, 2×200 mL) and brine (200 mL). The organic phase was dried (Na₂SO₄), the solids were removed by filtration, and the filtrate was concentrated in vacuo to afford Compound A-14 (10.37 g, 96.6% purity by HPLC, 46% yield) as a yellow oil.

¹H NMR (300 MHz, CDCl₃, ppm): d 4.21 (t, J=6.00 Hz, 4H), 4.04-4.17 (8H), 3.42 (t, J=6.00 Hz, 4H), 3.30 (t, J=6.90 Hz, 2H), 2.87 (s, 3H), 2.30-2.60 (8H), 2.10-2.27 (14H), 1.59 (m, 8H), 1.14-1.30 (40H), 0.88 (t, J=6.90 Hz, 12H); LCMS (+ mode): Calcd. for C₅₆H₁₀₃N₃O₁₃+H⁺: 1026.76. Found: 1027.00 [M+H⁺].

### Example 17. Synthesis of 2-(2-(tert-Butoxy)-2-oxoethyl)propane-1,3-diyl bis(decanoate) (I-c)

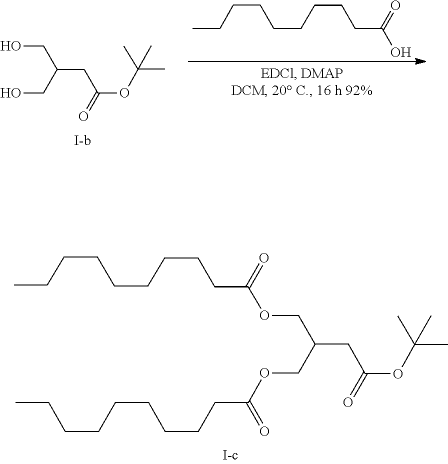

To a solution of I-b (30.0 g, 0.16 mol) in CH₂Cl₂ (300 mL), under nitrogen, was added in order decanoic acid (54.3 g, 0.32 mol) and DMAP (77.10 g, 0.63 mol). The mixture was stirred for 10 minutes at room temperature, then EDCI (66.7 g, 0.36 mol) was added in one portion. The mixture was allowed to stir for 16 h at room temperature then the solvent was removed in vacuo to provide crude I-c as a sticky, yellow oil. Crude I-c was dissolved in MTBE (450 mL) and the organic phase was extracted with 1-% aq. citric acid (2×300 mL). The combined aq. phases were extracted with MTBE (2×300 mL) and the combined organic phases were washed with brine (450 mL) and dried (Na₂SO₄). The solids were removed by filtration and the filtrate was concentrated in vacuo to give crude I-c as a yellow oil. Crude I-c was dissolved in ethyl acetate (250 mL), silica gel (90 g, type: ZCX-2, 100-200 mesh) was added to the solution. The solvent was removed in vacuo to give crude I-c impregnated on silica gel. The dry silica gel was placed onto a gravity column of silica gel (900 g, type: ZCX-2, 100-200 mesh, packed with petroleum ether), and the resulting column was eluted with a gradient of petroleum ether:ethyl acetate (100:0 to 95:5). Compound I-c eluted with petroleum ether:ethyl acetate 98:2 and the fractions of I-c were concentrated in vacuo to provide I-c as a pale pink oil (49.8 g, purity 98.6% by HPLC, 92% yield).

¹H NMR (400 MHz, CDCl₃, ppm): d 4.11 (m, 4H), 2.53 (m, 1H), 2.32 (m, 6H), 1.63 (m, 4H), 1.49 (s, 9H), 1.20-1.30 (24H), 0.90 (m, 6H).

### Example 18. Synthesis of 4-(Decanoyloxy)-3-((decanoyloxy)methyl)butanoic acid 1-d

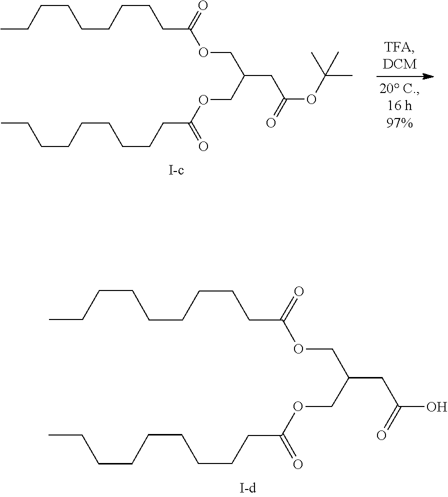

To a solution of I-c (49.0 g, 98.2 mmol) in CH₂Cl₂ (400 mL), at room temperature under nitrogen, was added trifluoroacetic acid (TFA) (147 mL, 1.92 mol) over 30 minutes. The resulting solution was stirred for 16 h at room temperature, then was concentrated in vacuo to provide crude I-d as a yellow oil. Crude I-d was dissolved in CH₂Cl₂ (500 mL) and the solution was washed with water (2×0250 L). The combined aq. phases were extracted with CH₂Cl₂ (0.25 L), and the combined organic layers were washed with 2% aq. NaHCO₃ (250 mL), and dried over anhydrous MgSO₄. Filtration and concentration in vacuo gave I-d as a an ivory solid (42.0 g, 95.6 mol, 99% yield, 98.5% purity by HPLC).

¹H NMR (400 MHz, CDCl₃, ppm): d 4.13 (m, 4H), 2.58 (m, 1H), 2.49 (d, J=6.90 Hz, 2H), 2.32 (t, J=7.60 Hz, 4H), 1.63 (m, 4H), 1.36-1.22 (24H), 0.89 (t, J=6.70 Hz, 6H); LCMS (+ mode): Calcd. for C₂₅H₄₆O₆+H⁺: 443.34. Found: 443.30.

### Example 19. Synthesis of (((((tert-butoxycarbonyl)azanediyl)bis(ethane-2,1-diyl))bis(oxy))bis(2-oxoethane-2,1-diyl))bis(propane-2,1,3-triyl) tetrakis(decanoate) I-e

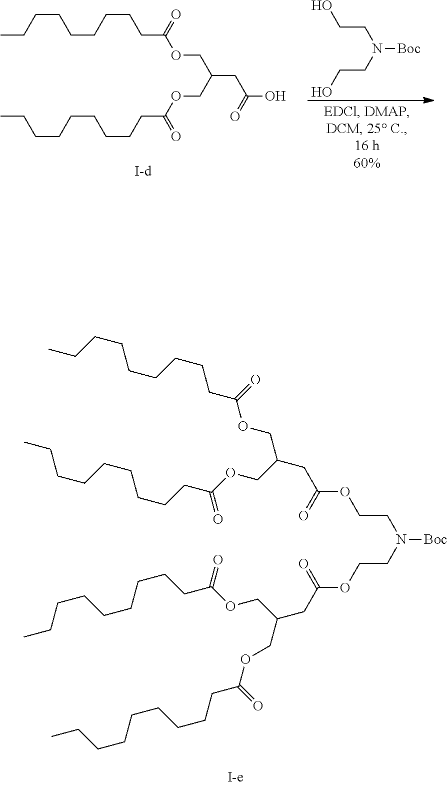 [SMILES: CCCCCCCCCC(=O)OCC(COC(=O)CCCCCCCCC)CC(=O)O ; CC(C)(C)OC(=O)N(CCO)CCO ; CCCCCCCCCC(=O)OCC(COC(=O)CCCCCCCCC)CC(=O)OCCN(CCOC(=O)CC(COC(=O)CCCCCCCCC)COC(=O)CCCCCCCCC)C(=O)OC(C)(C)C]

To a solution of tert-butyl bis(2-hydroxyethyl)carbamate (7.50 g, 36.6 mmol) in CH₂Cl₂ (225 mL), at room temperature under nitrogen was added in order I-d (34.00 g, 76.8 mmol) and DMAP (4.50 g, 36.6 mmol). The mixture was stirred for 10 minutes, then EDCI (17.60 gg, 91.5 mmol) was added over a period of 10 minutes, and the resulting solution was stirred for 16 hours at room temperature. The reaction mixture was washed with water (2×300 mL), the aq. phase was extracted with CH₂Cl₂ (2×300 mL) and the combined organic phases were dried over anhydrous MgSO₄. After filtration to remove the solids, silica gel (187.5 g, type: ZCX-2, 100-200 mesh) was added to the filtrate and the mixture was concentrated in vacuo to dryness. The dry silica gel was placed onto a gravity column of silica gel (1125 g, type: ZCX-2, 100-200 mesh, packed with heptane), and the resulting column was eluted with a gradient of heptane:THF (100:0 to 90:10). Compound I-e eluted with heptane:THF 95:5 and the fractions of I-e were concentrated in vacuo to provide I-e as a yellow oil (25.50 g, purity 91% by HPLC, 60% yield).

¹H NMR (400 MHz, CDCl₃, ppm): d 4.20 (m, 4H), 4.12 (m, 8H), 3.49 (m, 4H), 2.58 (m, 2H), 2.43 (d, J=7.00 Hz, 4H), 2.31 (t, J=7.60 Hz, 8H), 1.66-1.59 (8H), 1.48 (s, 9H), 1.21-1.39 (48H), 0.89 (m, 12H).

### Example 20. Synthesis of bis(2-((4-(decanoyloxy)-3-((decanoyloxy)methyl)butanoyl)oxy)ethyl)ammonium trifluoroacetate (I-f)

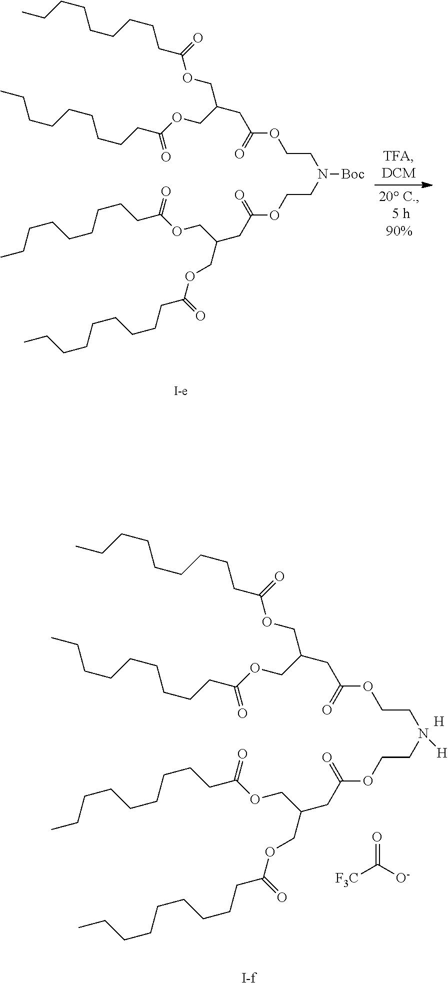

To a solution of I-e (27.00 g, 23.3 mmol) in CH₂Cl₂ (150 mL), at room temperature under nitrogen, was added TFA (54.0 mL, 0.71 mol) over a period of 30 minutes. After the addition was complete the mixture was allowed to stir for 5 hours, then the solution was washed with 10% aq. K₂HPO₄ (270 mL) and water (2×135 mL). The organic phase was dried (MgSO₄), filtered and concentrated in vacuo to give the ammonium salt I-f as a yellow oil (25.50 g, 88% purity by HPLC, 90% yield).

¹H NMR (400 MHz, CDCl₃, ppm): d 4.43 (m, 4H), 4.12 (m, 8H), 3.40 (m, 4H), 2.54 (m, 2H), 2.42 (m, 4H), 2.32 (m, 8H), 1.61 (m, 8H), 1.20-1.37 (48H), 0.89 (t, J=6.70 Hz, 12H); LCMS (+ mode): Calcd. for C₅₄H₁₀₀NO₁₂: 954.72. Found: 955.00.

### Example 21. Synthesis of (((((1H-imidazole-1-carbonyl)azanediyl)bis(ethane-2,1-diyl))bis(oxy))bis(2-oxoethane-2,1-diyl))bis(propane-2,1,3-triyl) tetrakis(decanoate) I-g

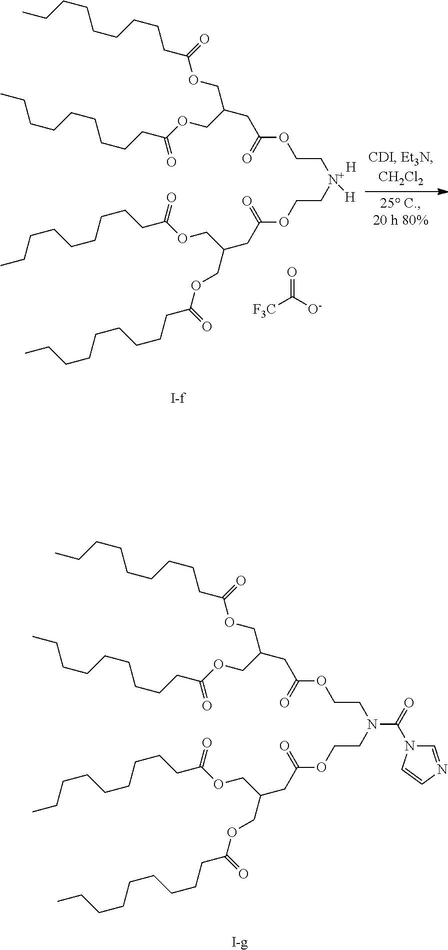

To a solution of I-f (25.00 g, 22.1 mmol), in CH₂Cl₂ (250 mL) at room temperature under nitrogen, was added in order: carbonyl diimidazole (CDI) (14.40 g, 88.6 mol) and Et₃N (4.50 g, 44.3 mol). The resulting solution was stirred for 3 h at room temperature. HPLC analysis indicated that the reaction was not complete, and additional CDI (14.4 g, 88.6 mmol) and Et₃N (4.50 g, 44.3 mol) were added. The solution was allowed to stir for 14 hours at room temperature, then the mixture was cast into aq. HCl (0.8M, 250 mL). The organic phase was separated, and the aq. phase was extracted with CH₂Cl₂ (250 mL). The combined organic phases were concentrated in vacuo to furnish crude I-g as a yellow oil which was dissolved in heptane (250 mL). The heptane solution was washed with MeOH—H₂O (5:1, 2×125 mL) and brine (125 mL). The heptane solution was dried (MgSO₄), the solids were removed by filtration and the filtrate was concentrated in vacuo to give I-g (23.40 g, purity by HPLC 79.8%, 80% yield) as a viscous, light yellow oil.

¹H NMR (400 MHz, CDCl₃, ppm): d 7.98 (s, 1H), 7.30 (s, 1H), 7.13 (s, 1H), 4.32 (m, 4H), 4.11 (m, 8H), 3.76 (m, 4H), 2.54 (m, 2H), 2.41 (d, J=6.90 Hz, 4H), 2.30 (m, 8H), 1.60 (m, 8H), 1.18-1.32 (48H), 0.89 (m, 12H); LCMS (+ mode): Calcd. for C₅₈H₁₀₁N₃O₁₃+H⁺: 1048.74. Found: 1049.10.

### Example 22. Synthesis of 1-(bis(2-((4-(decanoyloxy)-3-((decanoyloxy)methyl)butanoyl)oxy)ethyl)carbamoyl)-3-methyl-1H-imidazol-3-ium trifluoromethanesulfonate (I-h)

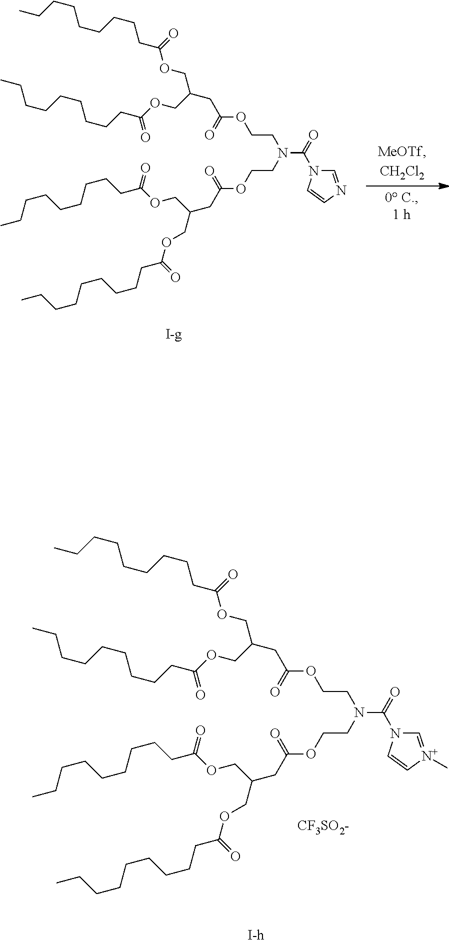

Acyl-imidazole 1-g (20.0 g, 18.2 mmol) was dissolved in CH₂Cl₂ (300 mL), under nitrogen, and was cooled in an ice water bath. To this cooled solution of 1-g was added MeOTf (4.50 g, 27.3 mmol) over a period of 15 minutes. The resulting solution was allowed to stir for 1 hour at 0° C., then was carried on to the target lipids (vide infra). HPLC and LCMS indicated complete consumption of 1-g.

LCMS (+ Mode): Calcd. for C₅₉H₁₀₄N₃O₁₃: 1062.76. Found: 1063.00.

### Example 23. Synthesis of Compound A-2

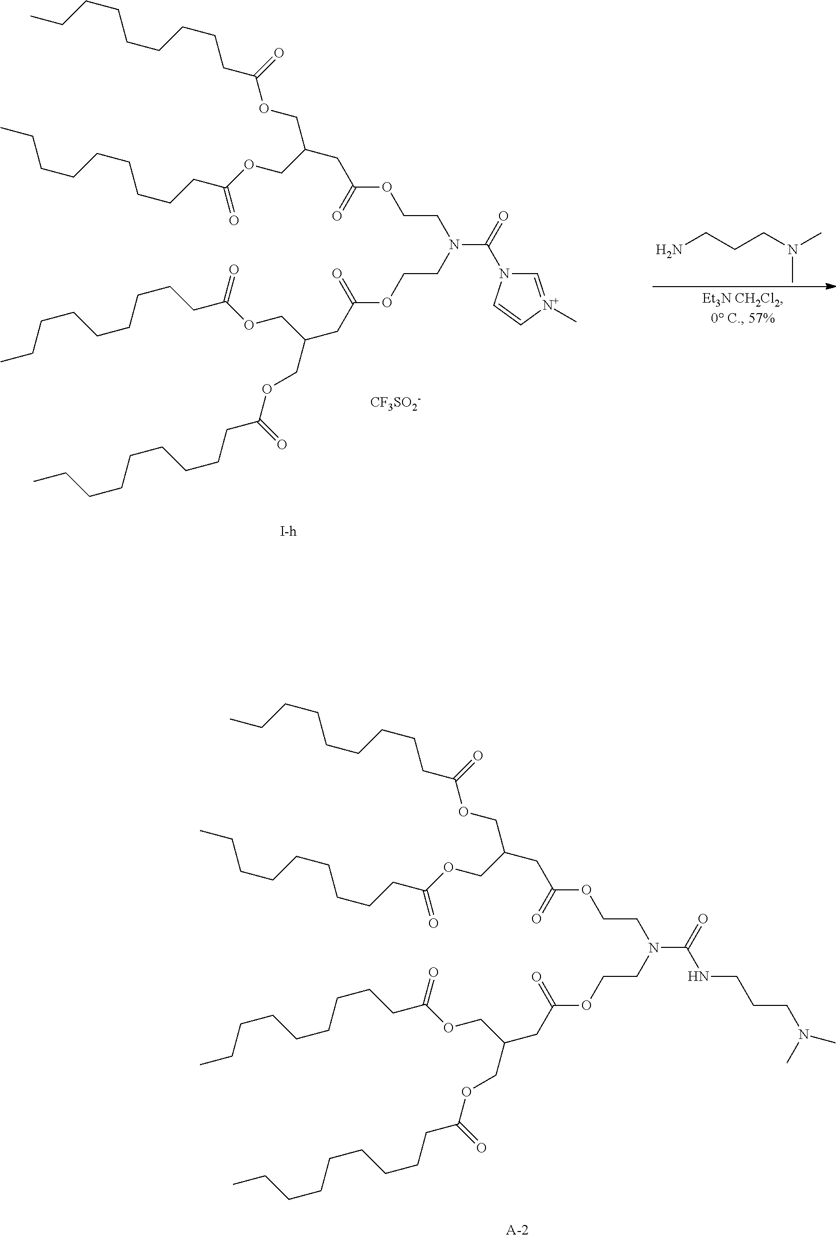

To a solution of 1-h, prepared as described above from 1-g (18.2 mmol), cooled in an ice-water bath under nitrogen, was added in order triethylamine (5.50 g, 54.7 mmol) and 3-dimethylaminopropylamine (2.80 g, 27.3 mmol). The mixture was stirred for 1 hour at 0° C., then was warmed to room temperature and stirred for 18 hours. The solution was concentrated in vacuo and the residue was dissolved in heptane (600 mL). The solution was washed with MeOH/H₂O (80:20, 2×150 mL), brine (150 mL) and dried (MgSO₄). The solids were removed by filtration, the filtrate was concentrated in vacuo to provide crude Compound A-2 as a viscous yellow oil. The crude product was dissolved in ethyl acetate (300 mL) and was washed with 5% aq. Na₂CO₃ (2×300 mL), brine (300 mL) and dried (MgSO₄). The solids were removed by filtration and silica gel (40 g, type: ZCX-2, 100-200 mesh) was added to the solution, and the mixture was concentrated in vacuo to dryness. The dry silica gel was placed onto a gravity column of silica gel (200 g, type: ZCX-2, 100-200 mesh, packed with CH₂Cl₂), and the resulting column was eluted with a gradient of CH₂Cl₂:MeOH (100:0 to 90:10). Compound A-2 eluted with CH₂Cl₂:MeOH 95:5 and the fractions of Compound A-2 were concentrated in vacuo to provide Compound A-2 as a yellow oil (12.5 g). Compound A-2 was dissolved in heptane (150 mL), washed with MeOH/H₂O (80:20, 2×150 mL), brine (150 mL) and dried (MgSO₄). The solids were removed by filtration, the filtrate was concentrated in vacuo to provide Compound A-2 as a pale, yellow oil (11.96 g, purity 94% by HPLC, 57% yield.

¹H NMR (400 MHz, CDCl₃, ppm): d 6.61 (m, 1H), 4.20 (t, J=6.40 Hz, 4H), 4.10 (m, 8H), 3.49 (t, J=6.00 Hz, 4H), 3.32 (m, 2H), 2.46-2.56 (4H), 2.41 (m, 4H), 2.23-2.32 (14H), 1.73 (m, 2H), 1.59 (m, 8H), 1.19-1.34 (48H), 0.89 (t, J=6.70 Hz, 12H60 LCMS (+ mode): Calcd. for C₅₆H₁₁₁N₃O₁₃+H⁺: 1082.82. Found: 1083.00 [M+H⁺].

### Example 24. Synthesis of Compound A-15

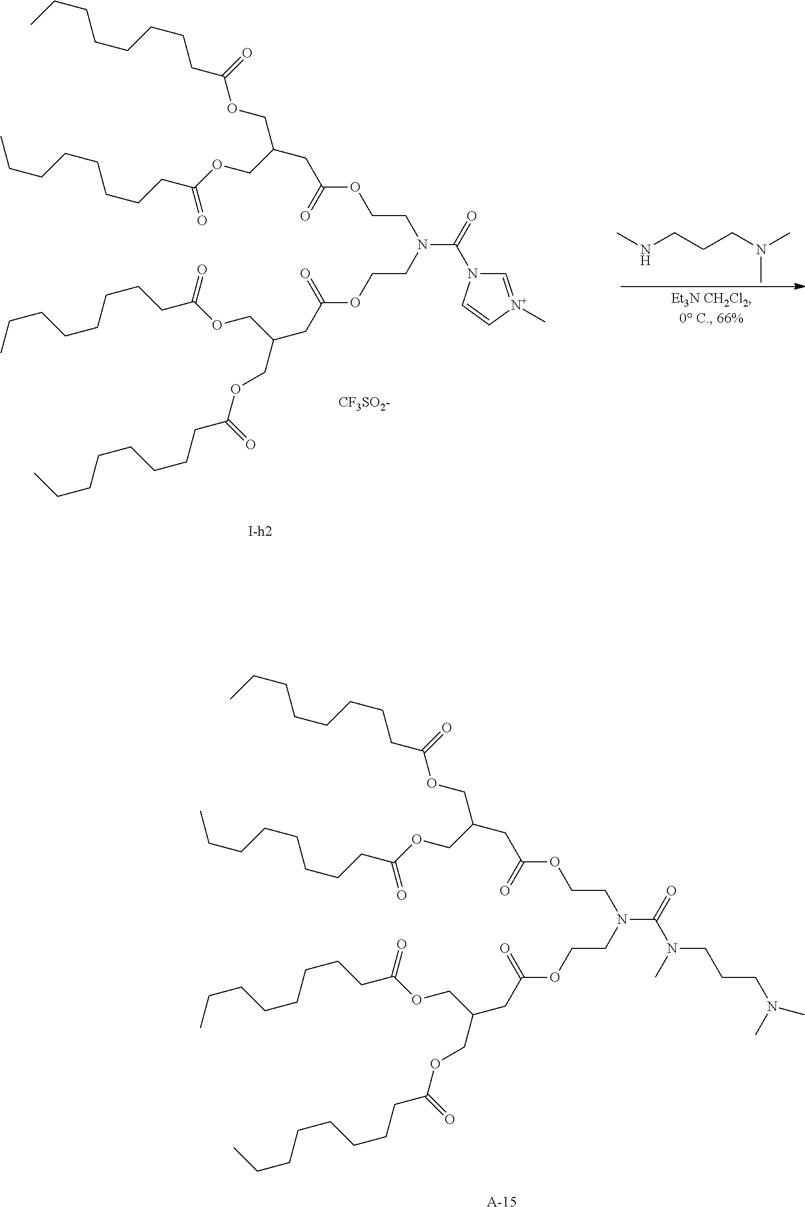

To a solution of I-h2, prepared as described above from I-g2 (15.12 mmol), cooled in an ice-water bath under nitrogen, was added in order triethylamine (4.60 g, 45.36 mmol) and N,N,N′-trimethypropylenediamine (2.63 g, 22.68 mmol). The mixture was stirred for 1 hour at 0° C., then was warmed to room temperature and stirred for 18 hours. The mixture was cast into water (750 mL) and the organic phase was separated. The aqueous phase was extracted with CH₂Cl₂ (2×300 mL), and the combined organic phases were washed with brine (300 mL) and dried (Na₂SO₄). The solids were removed by filtration and silica gel (30 g, type: ZCX-2, 100-200 mesh) was added to the filtrate, and the mixture was concentrated in vacuo to dryness. The dry silica gel was placed onto a gravity column of silica gel (150 g, type: ZCX-2, 100-200 mesh, packed with heptane), and the resulting column was eluted with a gradient of heptane:ethyl acetate (100:0 to 0:100). Compound A-15 eluted with heptane:ethyl acetate 70:30 and the fractions of Compound A-15 were concentrated in vacuo to provide Compound A-15 as a yellow oil (12.0 g, HPLC purity 88%). Compound A-15 was further purified by reverse phase flash chromatography (WelFlash XSelect CSH Prep C18, 5 mm OBD, Regular 30×150 mm column; Solvents: A: 0.05% formic acid in water, B: acetonitrile, gradient 50-80%, 20 minutes, flow 55 mL/min). Fractions containing Compound A-15 were pooled and concentrated in vacuo and the residue was dissolved in heptane (150 mL). The heptane solution was washed with MeOH/water (80:20, 2×100 mL) and brine (100 mL). The organic phase was dried (MgSO₄), the solids were removed by filtration, and the filtrate was concentrated in vacuo to afford Compound A-15 (10.33 g, 93% purity by HPLC, 66% yield) as a pale, yellow oil.

¹H NMR (300 MHz, CDCl₃, ppm): d 4.20 (t, J=6.00 Hz, 4H), 4.04-4.14 (8H), 3.41 (t, J=6.00 Hz, 4H), 3.20 (t, J=7.20 Hz, 2H), 2.83 (s, 3H), 2.52 (m, 2H), 2.20-2.40 (20H), 1.74 (m, 2H), 1.60 (m, 8H), 1.14-1.32 (40H), 0.90 (m, 12H); LCMS (+ mode): Calcd. for C₅₇H₁₀₅N₃O₁₃+H⁺: 1040.77. Found: 1040.90 [M+H⁺].

### Example 25. Synthesis of tert-Butyl (3-(2-methoxyethoxy)propyl)(methyl)carbamate (V-5a)

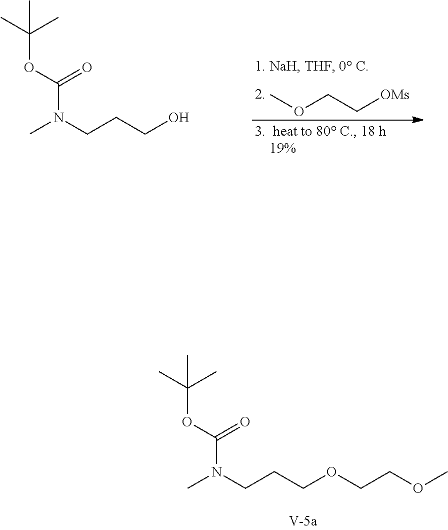

To a suspension of NaH (62% in oil, 10.1 g, 6.22 g, 0.259 mol) in THF (160 mL), cooled in an ice-water bath under nitrogen, was added a solution of tert-butyl (3-hydroxypropyl)(methyl)carbamate (40.0 g, 0.216 mol) in THF (160 mL) over 1 hour. The mixture was stirred for 1 hour after the addition was complete, then a solution of 2-methoxyethyl methanesulfonate (39.10 g, 0.254 mol) in THF (280 mL) was added over a period of 1 hour. After the addition was complete the mixture was warmed to 80° C. and was allowed to stir for 18 hours. The mixture was then cooled to room temperature and the reaction was quenched by the careful addition of sat'd. aq. NH₄Cl (500 mL) over 1 hour. The mixture was cast into ethyl acetate (500 mL), the organic phase was separated. The aqueous phase was extracted with ethyl acetate (2×500 mL) and the combined organic phases were washed with brine (1.5 L) and dried (Na₂SO₄). Filtration and concentration of the filtrate in vacuo gave crude V-5a (27.0 g) as a yellow liquid. The crude material was dissolved in CH₂Cl₂ (200 mL) and silica gel (50 g, type: ZCX-2, 100-200 mesh) was added to the filtrate, and the mixture was concentrated in vacuo to dryness. The dry silica gel was placed onto a gravity column of silica gel (500 g, type: ZCX-2, 100-200 mesh, packed and eluted with CH₂Cl₂). Fractions containing V-5a were concentrated in vacuo to provide V-5a as a yellow oil (10.0 g, 41.0 mmol, 19%).

¹H NMR (400 MHz, CD₃OD, ppm): d 3.33-3.73 (6H), 3.39 (s, 3H), 3.29 (t, J=7.00 Hz, 2H), 2.85 (s, 3H), 1.82 (m, 2H), 1.46 (s, 9H); LCMS (+ mode): Calcd. for C12H₂₅NO₄+H⁺: 248.19. Found: 248.20 [M+H⁺].

### Example 26. Synthesis of 3-(2-Methoxyethoxy)-N-methylpropan-1-aminium chloride (V-6a)

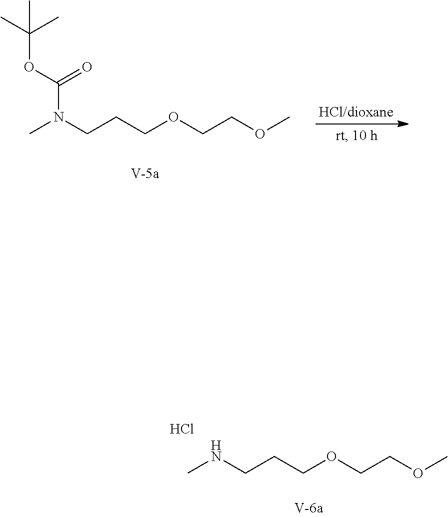

To as solution of V-5a (17.0 g, 68.7 mmol) in dioxane (350 mL), under nitrogen at room temperature, was added a solution of HCl in dioxane (2M, 350 mL) over a period of 30 minutes. The solution was stirred at room temperature for 10 hours, then the solvent was removed in vacuo to provide crude V-6a (13.0 g) as a yellow oil. Crude V-6a was utilized without purification.

LCMS (+ mode): Calcd. for C₇18₅NO₂: 148.13. Found: 148.30.

### Example 27. Synthesis of tert-Butyl (3-((3-(2-methoxyethoxy)propyl)(methyl)amino)propyl)(methyl)carbamate (V-7a)

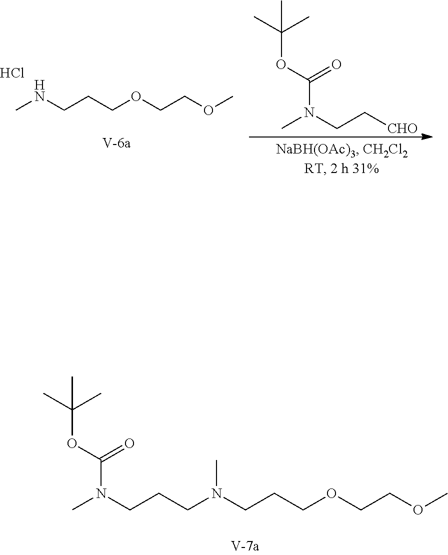

To a solution of V-6a (15.00 g, 81.7 mmol), in dichloromethane (180 mL) under nitrogen, was added tert-butyl methyl(3-oxopropyl)carbamate (20.99 g, 112 mmol) in one portion. The mixture was stirred for 30 minutes at room temperature, then NaBH(OAc)₃ (43.26 g, 204 mmol) was added in portions over 20 minutes. The solution was stirred at room temperature for 2 hours, then water (200 mL) was added, and the pH of the solution was adjusted to pH=8 by the addition of sat'd aq. Na₂CO₃. The mixture was extracted with CH₂Cl₂ (3×200 mL) and the combined organic phases were dried (Na₂SO₄). The solids were removed by filtration and silica gel (40 g, type: ZCX-2, 100-200 mesh) was added to the filtrate, and the mixture was concentrated in vacuo to dryness. The dry silica gel was placed onto a gravity column of silica gel (500 g, type: ZCX-2, 100-200 mesh, packed with CH₂Cl₂, eluted with a gradient of CH₂Cl₂:MeOH 100:0 to 90:10). Fractions containing V-7a (CH₂Cl₂:MeOH 95:5) were concentrated in vacuo to provide V-7a as a yellow oil (8.0 g, 25.1 mmol, 31%).

¹H NMR (400 MHz, CDCl₃, ppm): d 3.20-3.65 (14H), 2.86 (m, 4H), 1.62-1.85 (6H), 1.48 (s, 9H); LCMS (+ mode): Calcd. for C₁₆H₃₄N₂O₄+H⁺: 319.26. Found: 319.40 [M+H⁺].

### Example 28. Synthesis of 3-((3-(2-Methoxyethoxy)propyl)(methyl)amino)-N-methylpropan-1-aminium chloride (V-8a)

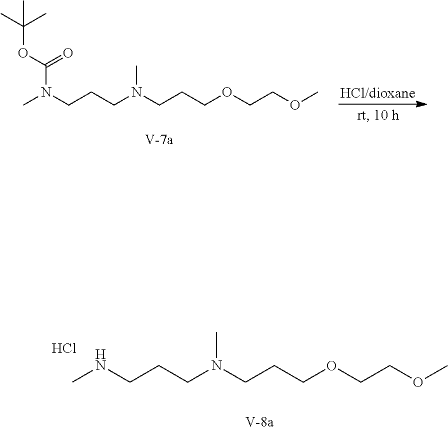

To a solution of V-7a (1.00 g, 3.14 mmol) in dioxane (20 mL), at room temperature under nitrogen, was added HCl in dioxane (2M, 20 mL) over 5 minutes. The resulting solution was stirred at room temperature for 10 hours, then the solvent was removed in vacuo to afford crude V-8a (800 mg) as a white solid. Crude 18 was utilized without further purification.

¹H NMR (400 MHz, CDCl₃, ppm): d 10.92 (brs, 1H), 9.75 (brs, 2H), 3.00-3.62 (15H), 2.94 (brs, 3H), 2.76 (brs, 3H), 2.53 (brm, 2H), 2.17 (brm, 2H); LCMS (+ mode): Calcd. for C₁₁H₂₇N₂O₂: 219.21. Found: 219.20.

### Example 29. Synthesis of bis(2-((tert-Butyldimethylsilyl)oxy)ethyl)amine (V-12)

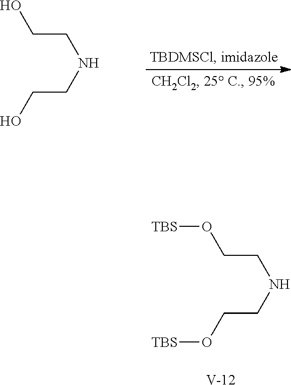

To a solution of diethanolamine (100.00 g, 0.952 mol) in dichloromethane (1.0 L), at room temperature under nitrogen, was added imidazole (194.0 g, 2.86 mol), and the resulting solution was stirred for 5 minutes. To this mixture was added a solution of t-butyldimethlylsilylchloride (316.3 g, 2.10 mol) in dichloromethane (1.0 L) over a period of 30 minutes. The resulting solution was stirred for 2 hours at room temperature, then the reaction was quenched by the addition 10% aq. NH₄OH (400 mL). The organic phase was separated, the aq. phase was extracted with dichloromethane (2×600 mL), and the combined organic phases were washed with sat'd. aq. NH₄Cl (5×800 mL), brine (800 mL), and dried (Na₂SO₄). Filtration and concentration in vacuo afforded V-12 (300.0 g, 0.899 mol, 94%) as a clear, colorless oil.

¹H NMR (400 MHz, CDCl₃, ppm): d 3.75 (t, J=5.30 Hz, 4H), 2.74 (t, J=5.30 Hz, 4H), 2.03 (brs, 1H), 0.91 (s, 18H), 0.07 (s, 12H); LCMS (+ mode): Calcd. for C₁₆H₁₉NO₂Si₂+H⁺: 334.26. Found: 334.40.

### Example 30. Synthesis of N,N-bis(2-((tert-Butyldimethylsilyl)oxy)ethyl)-1H-imidazole-1-carboxamide (V-13)

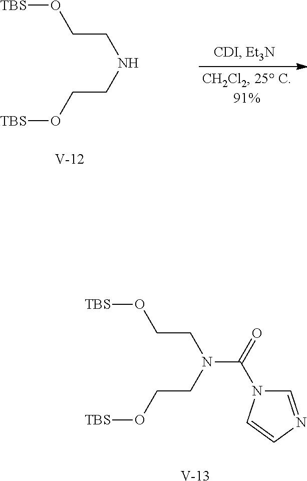

To a solution of V-12 (230.0 g, 0.690 mol) in dichloromethane (2.30 L), at room temperature under nitrogen, was added in order CDI (446.20 g, 2.75 mol) and Et₃N (139.10 g, 1.38 mol). The resulting solution was allowed to stir for 16 hours at room temperature, then the mixture was cast into water (2.30 L). The organic phase was separated, the aq. layer was extracted with dichloromethane (1.15 L) and the combined organic phases were washed with sat'd. aq. NH₄Cl (2×4.6 L), 5% aq. NaHCO₃ (4.6 L), and dried (Na₂SO₄). Filtration and concentration in vacuo afforded crude V-13 as a yellow oil which was dissolved in heptane (250 mL). The solution was washed with MeOH/H₂O (80:20, 1.15 L) and dried (MgSO₄). Filtration and concentration in vacuo gave V-13 (270.0 g, 0.631 mol, 91%) as a pale, yellow oil.

¹H NMR (400 MHz, CDCl₃, ppm): d 8.06 (t, J=1.10 Hz, 1H), 7.42 (t, J=1.40 Hz, 1H), 7.07 (dd, J=1.40, 1.10 Hz, 1H), 3.85 (t, J=5.30 Hz, 4H), 3.64 (t, J=5.30 Hz, 4H), 0.89 (s, 18H), 0.08 (s, 12H); LCMS (+ mode): Calcd. for C₂₀H₄₁N₃O₃Si₂+H⁺: 428.28. Found: 428.30.

### Example 31. Synthesis of 1,1-bis(2-((tert-butyldimethylsilyl)oxy)ethyl)-3-(3-((3-(2-methoxyethoxy)propyl) (methyl)amino)propyl)-3-methylurea (V-14)

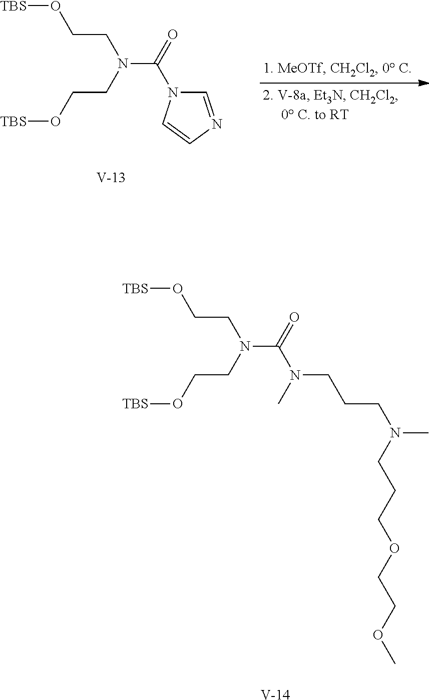

To a solution of V-13 (20.0 g, 48 mmol) in dichloromethane (200 mL), cooled in an ice-water bath under nitrogen, was added MeOTf (8.40 g, 51.0 mmol) over a period of 5 minutes. The resulting mixture was allowed to stir at 0° C. for 1 hour, then a solution of Et₃N (14.0 g, 140 mmol) and V-8a (17.80 g, 70 mmol) in dichloromethane (200 mL) was added to the solution over 30 minutes. After the addition was complete, the mixture was warmed to room temperature and was stirred for 16 hours. The reaction mixture was cast into water (200 mL) and the organic phase was removed. The aqueous layer was extracted with dichloromethane (2×200 mL) and the combined organic phases were concentrated in vacuo. The resulting crude V-14 was dissolved in heptane (300 mL) and the solution was extracted with MeOH/H₂O (75:25, 2×100 mL). The combined aqueous phases were extracted with heptane (6×200 mL), and the combined organic phases were washed with brine (400 mL). The organic phase was dried (MgSO₄). After filtration, silica gel (60 g, type: ZCX-2, 100-200 mesh) was added to the filtrate, and the mixture was concentrated in vacuo to dryness. The dry silica gel was placed onto a gravity column of silica gel (330 g, type: ZCX-2, 100-200 mesh, packed with CH₂Cl₂, eluted with a gradient of CH₂Cl₂/MeOH 100:0 to 90:10). Fractions containing V-14 (CH₂Cl₂/MeOH 93:7) were concentrated in vacuo to provide V-14 as a yellow oil (11.15 g, 22.0 mmol, 46%).

¹H NMR (400 MHz, CDCl₃, ppm): d 3.72 (t, J=6.20 Hz, 4H), 3.48-3.60 (6H), 3.40 (s, 3H), 3.33 (t, J=6.20 Hz, 4H), 3.17 (t, J=7.50 Hz, 2H), 2.83 (s, 3H), 2.31-2.50 (4H), 2.26 (brs, 3H), 1.70-1.83 (4H), 0.90 (s, 18H), 0.06 (s, 12H); LCMS (+ mode): Calcd. for C₂₈H₆₃N₃O₅Si₂+H⁺: 578.44. Found: 578.30.

### Example 32. Synthesis of 1,1-bis(2-hydroxyethyl)-3-(3-((3-(2-methoxyethoxy)propyl)(methyl)amino)propyl)-3-methylurea (V-15)

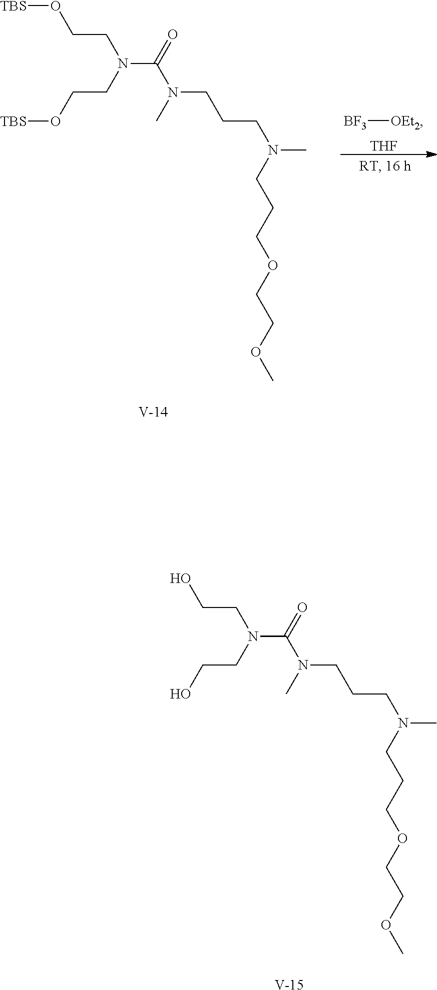

To a solution of V-14 (11.15 g, 22.0 mmol) in THF (125 mL), under nitrogen at room temperature, was added BF₃-OEt₂ (8.20 mL, 9.40 g, 66.0 mmol) over a period of 10 minutes. The mixture was stirred for 16 hours at room temperature, then was poured onto water (100 mL). The pH of the solution was adjusted to pH=8.0 by the addition of sat'd. aq. NaHCO₃ and the solvent was removed in vacuo to ca. 25 mL volume. The remaining solution was purified by reverse phase flash chromatography (WelFlash AQ-C18 gel, regular 120 g, A: water, B: acetonitrile, gradient 0-30% over 15 minutes, Flow: 80 mL/min). Target V-15 eluted at 30% acetonitrile and fractions containing V-15 were pooled and concentrated in vacuo to provide V-15 as an off-white oil (7.38 g, 21.12 mmol, 96% yield).

¹H NMR (400 MHz, CDCl₃, ppm): d 4.50 (brs, 2H), 3.72 (m, 4H), 3.41-3.57 (6H), 3.28-3.38 (9H), 2.82 (s, 3H), 2.48 (m, 2H), 2.39 (t, J=6.60 Hz, 2H), 2.25 (s, 3H), 1.70-1.85 (4H); LCMS (+ mode): Calcd. for C₁₆H₃₅N₃O₅+H⁺: 350.27. Found: 350.40.

### Example 33. Synthesis of Compound A-16

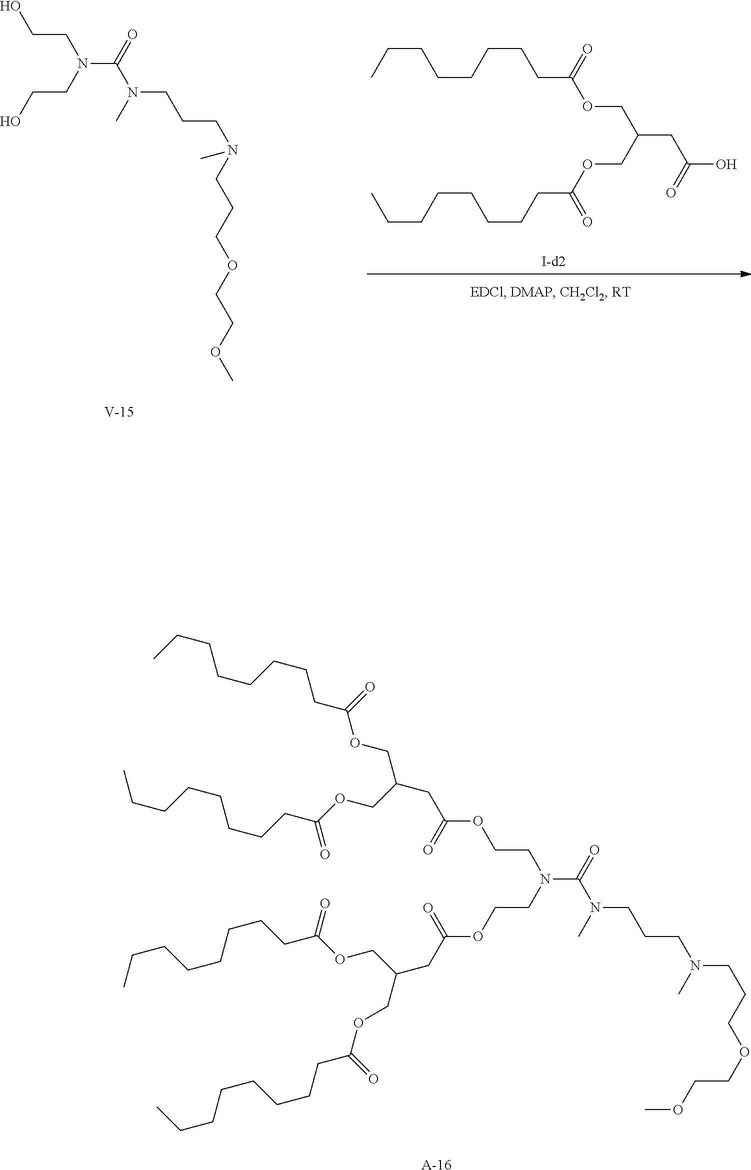

To a solution of V-15 (6.50 g, 18.6 mmol) in dichloromethane (130 mL), under nitrogen at room temperature, was added in order I-d2 (17.00 g, 40.9 mmol), DMAP (2.30 g, 18.6 mmol), and EDCI (8.20 g, 42.7 mmol). The resulting solution was stirred for 16 h at room temperature, then was cast into water (100 mL). The organic phase was separated, the aq. phase was extracted with dichloromethane (2×100 mL) and the combined organic phases were concentrated in vacuo. The resulting crude Compound A-16 was dissolved in heptane (150 mL) and the resulting solution was washed with MeOH/water (80:20, 100 mL), brine (100 mL), and dried (Na₂SO₄). The solids were removed by filtration and silica gel (25 g, type: ZCX-2, 100-200 mesh) was added to the filtrate, and the mixture was concentrated in vacuo to dryness. The dry silica gel was placed onto a gravity column of silica gel (175 g, type: ZCX-2, 100-200 mesh, packed with CH₂Cl₂, eluted with a gradient of CH₂Cl₂/MeOH 100:0 to 90:10). Fractions containing Compound A-16 (CH₂Cl₂/MeOH 95:5) were concentrated in vacuo to provide Compound A-16 (94% purity by HPLC) as a yellow oil. Compound A-16 was further purified by reverse phase flash chromatography (WelFlash XSelect CSH Prep C18, 5 mm OBD, Regular 30×150 mm column; Solvents: A: 0.1% formic acid in water, B: acetonitrile, gradient 50-80%, 20 minutes, flow 55 mL/min). Fractions containing Compound A-16 were pooled, and concentrated in vacuo and the residue was dissolved in heptane (150 mL). The heptane solution was washed with 5% aq. Na₂CO₃ (2×100 mL), MeOH/water (75:25, 2×100 mL) and brine (100 mL). The organic phase was dried (Na₂SO₄), the solids were removed by filtration, and the filtrate was concentrated in vacuo to afford Compound A-16 (12.08 g, 96.4% purity by HPLC, 51% yield) as a yellow oil.

¹H NMR (400 MHz, CDCl₃, ppm): d 4.19 (t, J=6.00 Hz, 4H), 4.08 (m, 8H), 3-46-3.60 (6H), 3.41 (t, J=6.00 Hz, 4H), 3.37 (s, 3H), 3.17 (m, 2H), 2.82 (s, 3H), 2.53 (m, 2H), 2.35-2.42 (6H), 2.24-2.33 (10H), 2.19 (s, 3H), 1.66-1.80 (4H), 1.54-1.65 (8H), 1.20-1.36 (40H), 0.89 (m, 12H); LCMS (+ mode): Calcd. for C₆₂H₁₁₅N₃O₁₅+H⁺: 1142.84. Found: 1142.90.

### Example 34. Biophysical and Biochemical Characterization

Biophysical and biochemical characteristics of c log D, c-pKa, pKa, and ex vivo stability in mouse plasma were determined for Compounds A-2 and A-11 thru A-15 as well as for three benchmark lipids known to successfully deliver nucleic acids into cells, 10a, 10f, and 10p (see Journal of Medicinal Chemistry 63:12992-13012, 2020).

TABLE 5

| | | | | | ex vivo |
|---|---|---|---|---|---|
| | | | | | Mouse Plasm |
| | | | | | Stability T½ |
| | | | | | or % Remaining |
| Lipid | cLogD* | c-pKa* | pKa | ΔpKa** | at 2 Hours |
| 10a | 11.32 | 8.68 | 6.09 | 2.59 | 6.9 min^{#} |
| 10f | 10.91 | 8.68 | 6.21 | 2.47 | 73%^{#} |
| 10p | 13.70 | 9.34 | 6.50 | 2.84 | 100%^{#} |
| A-2 | 13.92 | 9.54 | 7.92 | 1.62 | 72% |
| A-11 | 11.95 | 8.47 | 6.66 | 1.81 | 86% |
| A-12 | 13.46 | 9.26 | 7.35 | 1.91 | 85% |
| A-13 | 12.68 | 8.36 | 7.51 | 0.80 | 64% |
| A-14 | 13.94 | 8.37 | 7.46 | 0.91 | 61% |
| A-15 | 13.16 | 9.55 | 8.37 | 1.18 | 69% |

*Calculated using ACD Labs Structure Designer v12.0. The cLogP component of cLogD was calculated using ACD Labs version B; cLogD was calculated at pH = 7.4.
**difference of calculated and measured pKa
^{#}Journal of Medicinal Chemistry 63: 12992-13012, 2020 which is incorporated by reference for all that it teaches about ionizable cationic lipids and LNP comprising them that does not contradict or is not inconsistent with this disclosure.

c Log D and c-pKa were calculated as noted above. The measured pKa of a lipid was determined as formulated in a lipid nanoparticle using the TNS assay as described in the following Example.

Past experience leads to the expectation that the difference between c-pKa and the measured pKa in an LNP (ΔpKa) will be between 2 and 3 units; however, all of Compounds A-2 and A-11 thru A-15 surprisingly had a ΔpKa of less than 2. The activity of ionizable amino lipids for promoting endosomal escape of the nucleic acid cargo is typically greatest for lipids with a pKa of between 6 and 7. Of the disclosed lipids tested in this Example, only Compound A-11 had an observed pKa in this range. One way to reduce the measured basicity of these lipids toward and into the preferred range for good endosomal escape activity is to increase the chain length of the fatty acid tails (R of Formula I) each by 1 to 4 carbons. Table 6 shows the structure of analogs of Compounds A-2 and A-12 thru A-15 along with their calculated c Log D and c-pKa with lengthened R groups (C₁₀-C₁₃ for Compound A-2 and C₉-C₁₂ for Compounds A-12 thru A-15. In each case, c Log D increased reflecting the increase in lipophilicity as the length of R is increased, but c-pKa remained the same. However, the increased lipophilicity will lead to a decrease in the measured pKa of the lipid when incorporated into an LNP and an increase in ΔpKa.

TABLE 6

| Lipid | Analog | c-LogD | c-pKa |
|---|---|---|---|
| 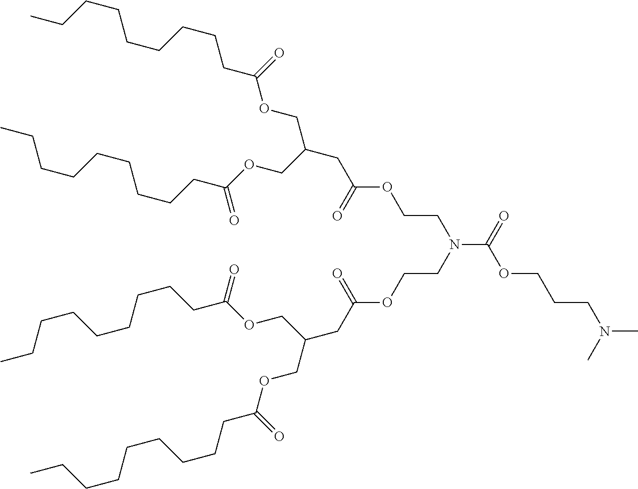 [SMILES: CCCCCCCCCC(=O)OCC(COC(=O)CCCCCCCCC)CC(=O)OCCN(CCOC(=O)CC(COC(=O)CCCCCCCCC)COC(=O)CCCCCCCCC)C(=O)OCCCN(C)C] | A-12R9 | 15.50 | 9.26 |
| 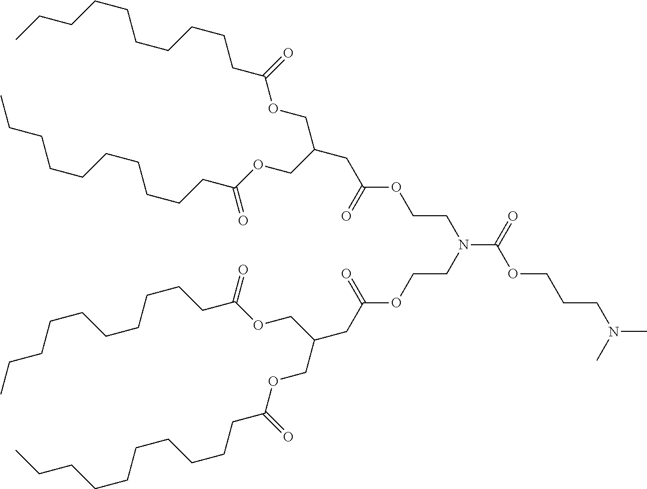 [SMILES: CCCCCCCCCCC(=O)OCC(COC(=O)CCCCCCCCCC)CC(=O)OCCN(CCOC(=O)CC(COC(=O)CCCCCCCCCC)COC(=O)CCCCCCCCCC)C(=O)OCCCN(C)C] | A-12R10 | 17.54 | 9.26 |
| 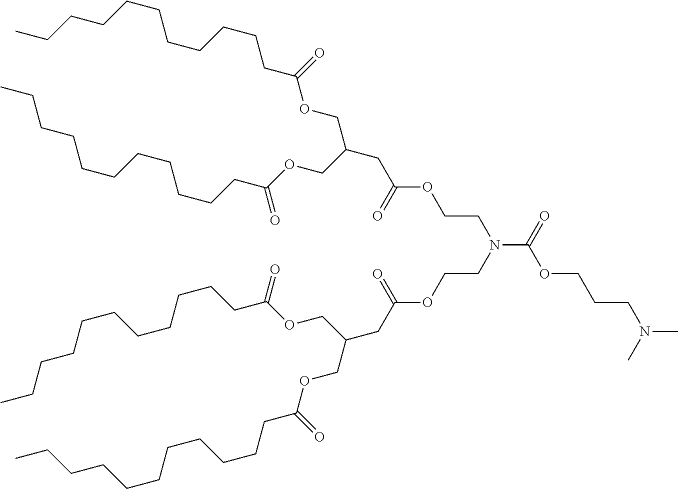 [SMILES: CCCCCCCCCCCC(=O)OCC(COC(=O)CCCCCCCCCCC)CC(=O)OCCN(CCOC(=O)CC(COC(=O)CCCCCCCCCCC)COC(=O)CCCCCCCCCCC)C(=O)OCCCN(C)C] | A-12R11 | 19.58 | 9.26 |
| 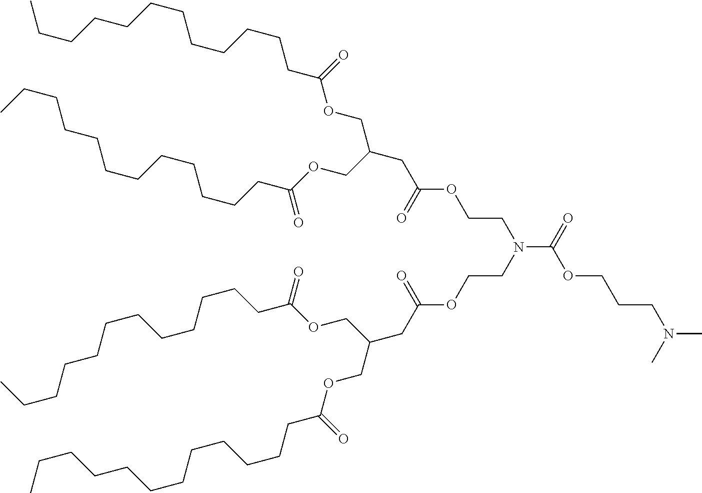 [SMILES: CCCCCCCCCCCCC(=O)OCC(COC(=O)CCCCCCCCCCCC)CC(=O)OCCN(CCOC(=O)CC(COC(=O)CCCCCCCCCCCC)COC(=O)CCCCCCCCCCCC)C(=O)OCCCN(C)C] | A-12R12 | 21.61 | 9.26 |
| 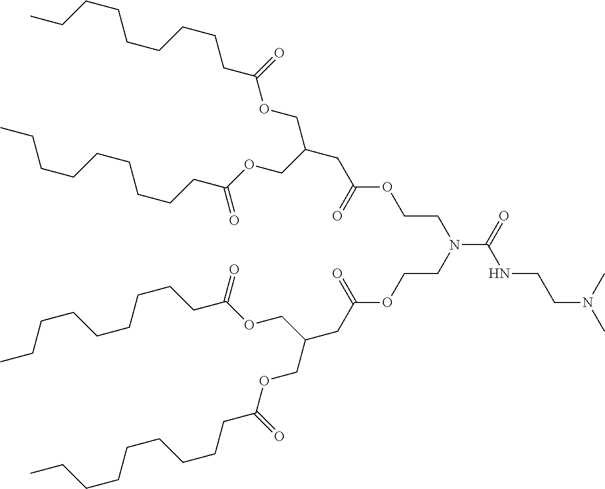 [SMILES: CCCCCCCCCC(=O)OCC(COC(=O)CCCCCCCCC)CC(=O)OCCN(CCOC(=O)CC(COC(=O)CCCCCCCCC)COC(=O)CCCCCCCCC)C(=O)NCCN(C)C] | A-13R9 | 14.71 | 8.36 |
| 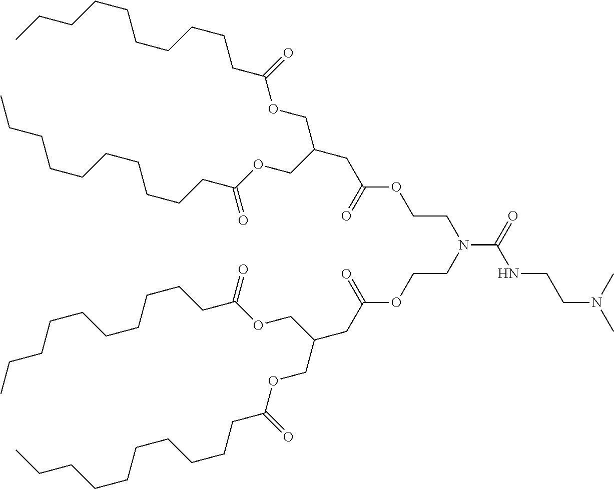 [SMILES: CCCCCCCCCCC(=O)OCC(COC(=O)CCCCCCCCCC)CC(=O)OCCN(CCOC(=O)CC(COC(=O)CCCCCCCCCC)COC(=O)CCCCCCCCCC)C(=O)NCCN(C)C] | A-13R10 | 16.75 | 8.36 |
| 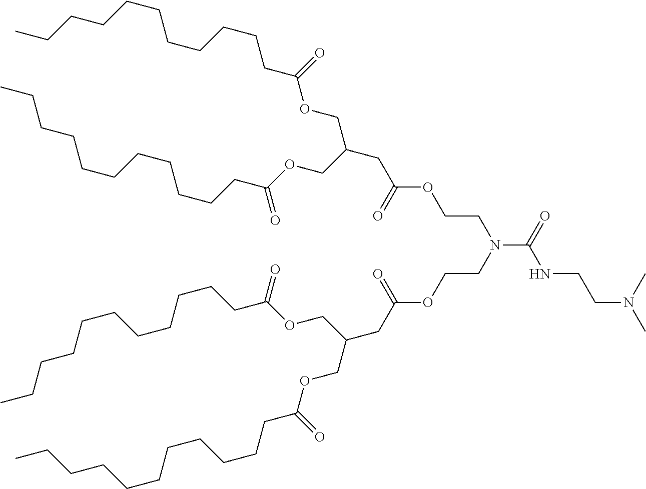 [SMILES: CCCCCCCCCCCC(=O)OCC(COC(=O)CCCCCCCCCCC)CC(=O)OCCN(CCOC(=O)CC(COC(=O)CCCCCCCCCCC)COC(=O)CCCCCCCCCCC)C(=O)NCCN(C)C] | A-13R11 | 18.79 | 8.36 |
| 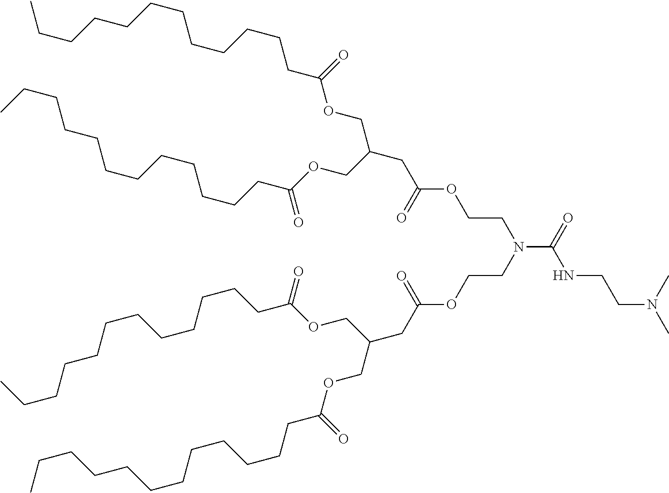 [SMILES: CCCCCCCCCCCCC(=O)OCC(COC(=O)CCCCCCCCCCCC)CC(=O)OCCN(CCOC(=O)CC(COC(=O)CCCCCCCCCCCC)COC(=O)CCCCCCCCCCCC)C(=O)NCCN(C)C] | A-13R12 | 20.83 | 8.36 |
| 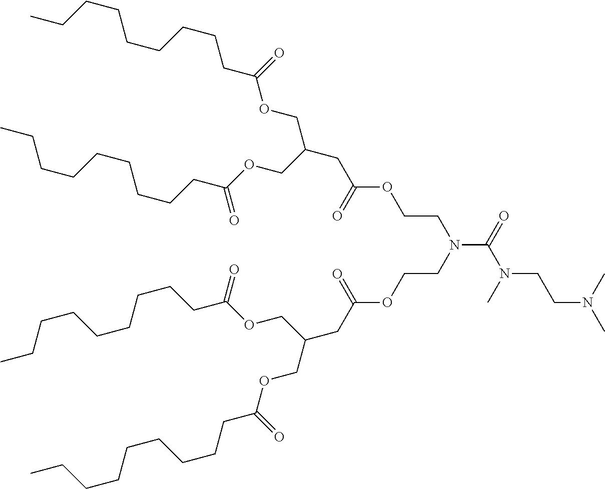 [SMILES: CCCCCCCCCC(=O)OCC(COC(=O)CCCCCCCCC)CC(=O)OCCN(CCOC(=O)CC(COC(=O)CCCCCCCCC)COC(=O)CCCCCCCCC)C(=O)N(C)CCN(C)C] | A-14R9 | 16.49 | 8.37 |
| 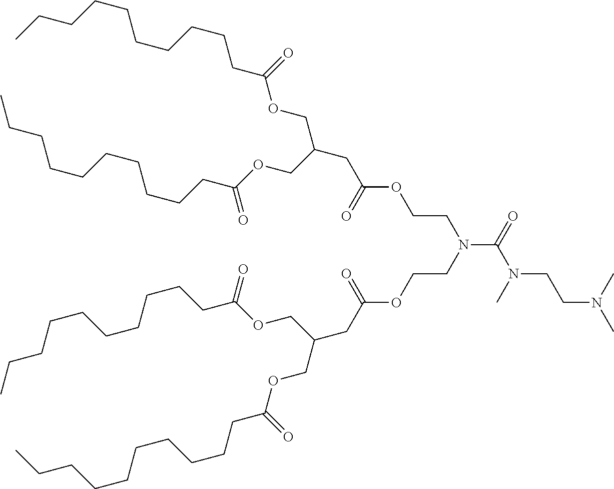 [SMILES: CCCCCCCCCCC(=O)OCC(COC(=O)CCCCCCCCCC)CC(=O)OCCN(CCOC(=O)CC(COC(=O)CCCCCCCCCC)COC(=O)CCCCCCCCCC)C(=O)N(C)CCN(C)C] | A-14R10 | 18.01 | 8.37 |
| 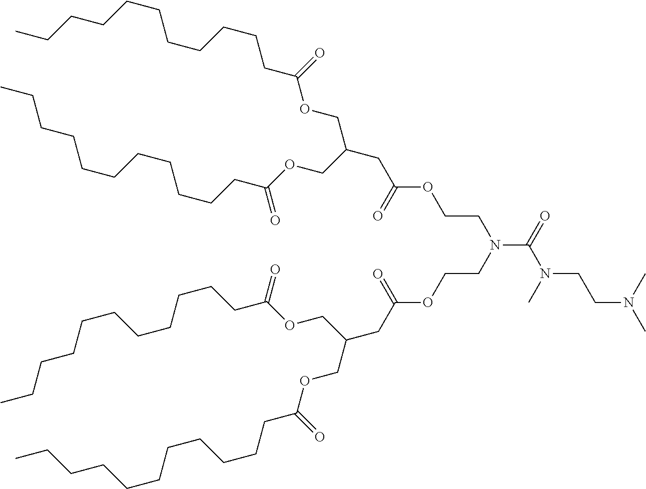 [SMILES: CCCCCCCCCCCC(=O)OCC(COC(=O)CCCCCCCCCCC)CC(=O)OCCN(CCOC(=O)CC(COC(=O)CCCCCCCCCCC)COC(=O)CCCCCCCCCCC)C(=O)N(C)CCN(C)C] | A-14R11 | 20.05 | 8.37 |
| 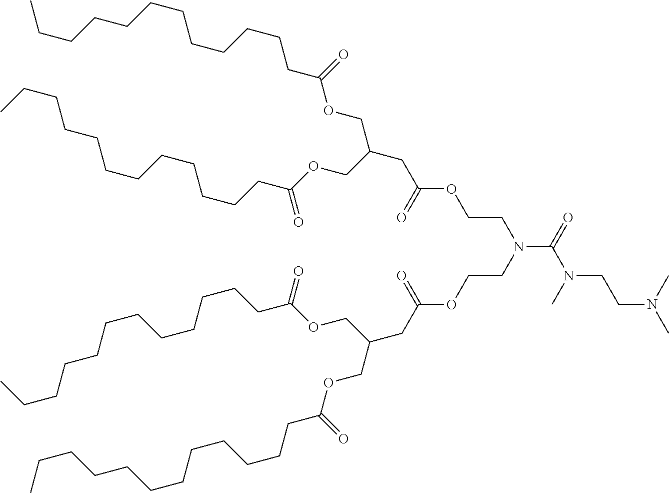 [SMILES: CCCCCCCCCCCCC(=O)OCC(COC(=O)CCCCCCCCCCCC)CC(=O)OCCN(CCOC(=O)CC(COC(=O)CCCCCCCCCCCC)COC(=O)CCCCCCCCCCCC)C(=O)N(C)CCN(C)C] | A-14R12 | 22.09 | 8.37 |
| 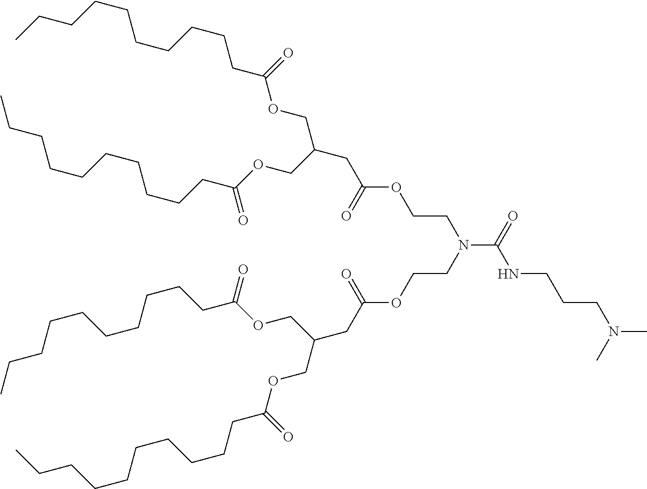 [SMILES: CCCCCCCCCCC(=O)OCC(COC(=O)CCCCCCCCCC)CC(=O)OCCN(CCOC(=O)CC(COC(=O)CCCCCCCCCC)COC(=O)CCCCCCCCCC)C(=O)NCCCN(C)C] | A-2R10 | 15.96 | 9.54 |
| 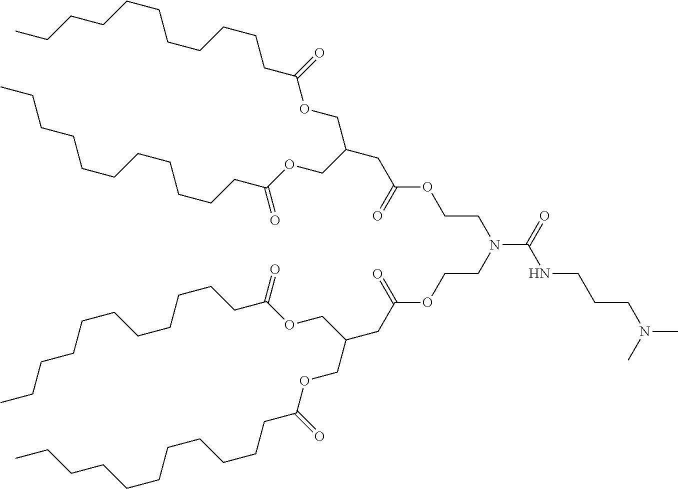 [SMILES: CCCCCCCCCCCC(=O)OCC(COC(=O)CCCCCCCCCCC)CC(=O)OCCN(CCOC(=O)CC(COC(=O)CCCCCCCCCCC)COC(=O)CCCCCCCCCCC)C(=O)NCCCN(C)C] | A-2R11 | 17.99 | 9.54 |
| 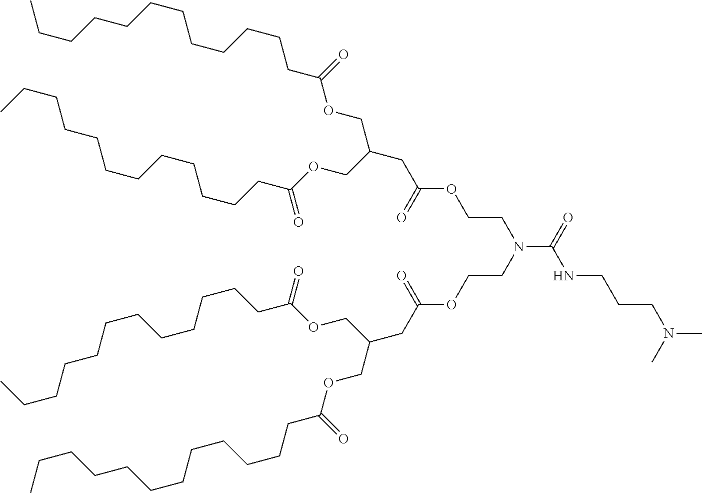 [SMILES: CCCCCCCCCCCCC(=O)OCC(COC(=O)CCCCCCCCCCCC)CC(=O)OCCN(CCOC(=O)CC(COC(=O)CCCCCCCCCCCC)COC(=O)CCCCCCCCCCCC)C(=O)NCCCN(C)C] | A-2R12 | 20.03 | 9.54 |
| 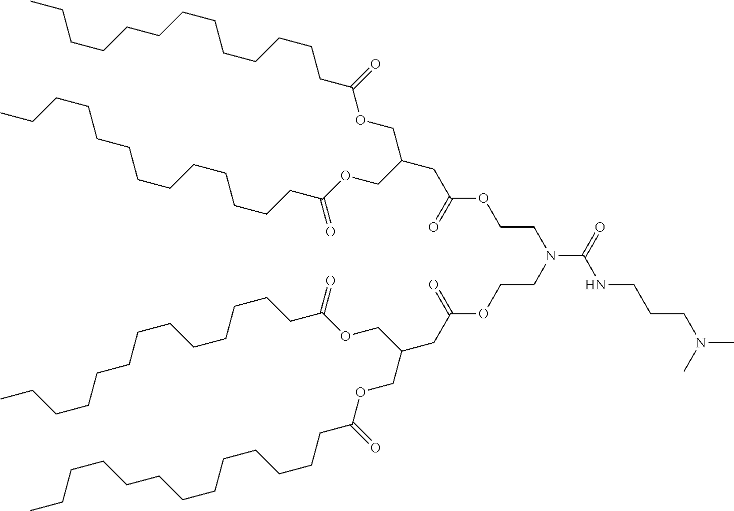 [SMILES: CCCCCCCCCCCCCC(=O)OCC(COC(=O)CCCCCCCCCCCCC)CC(=O)OCCN(CCOC(=O)CC(COC(=O)CCCCCCCCCCCCC)COC(=O)CCCCCCCCCCCCC)C(=O)NCCCN(C)C] | A-2R13 | 22.07 | 9.54 |
| 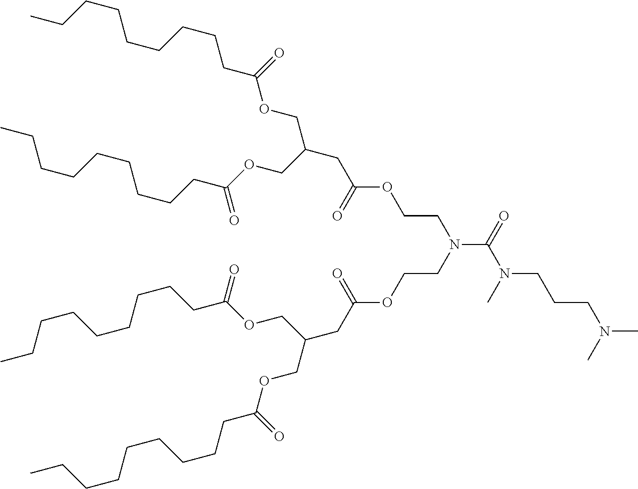 [SMILES: CCCCCCCCCC(=O)OCC(COC(=O)CCCCCCCCC)CC(=O)OCCN(CCOC(=O)CC(COC(=O)CCCCCCCCC)COC(=O)CCCCCCCCC)C(=O)N(C)CCCN(C)C] | A-15R9 | 15.20 | 9.55 |
| 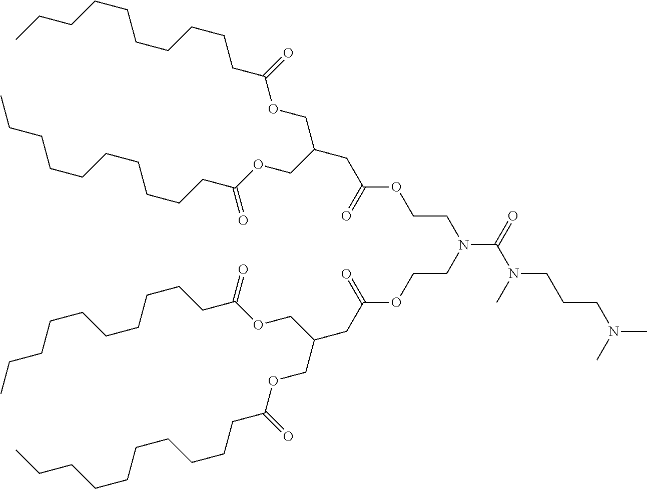 [SMILES: CCCCCCCCCCC(=O)OCC(COC(=O)CCCCCCCCCC)CC(=O)OCCN(CCOC(=O)CC(COC(=O)CCCCCCCCCC)COC(=O)CCCCCCCCCC)C(=O)N(C)CCCN(C)C] | A-15R10 | 17.23 | 9.55 |
| 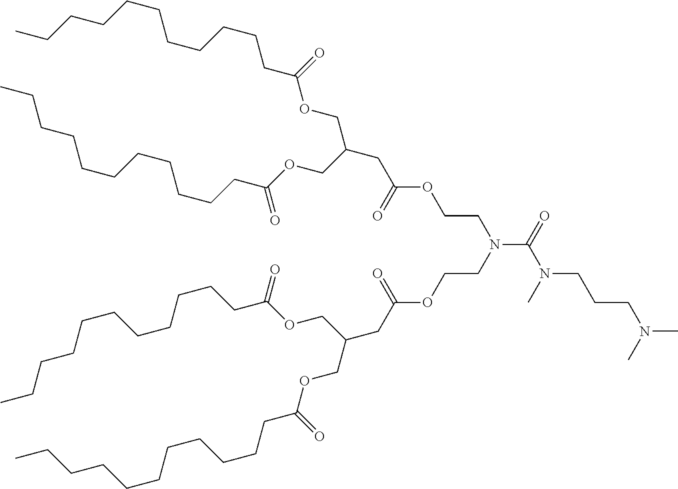 [SMILES: CCCCCCCCCCCC(=O)OCC(COC(=O)CCCCCCCCCCC)CC(=O)OCCN(CCOC(=O)CC(COC(=O)CCCCCCCCCCC)COC(=O)CCCCCCCCCCC)C(=O)N(C)CCCN(C)C] | A-15R11 | 19.27 | 9.55 |
| 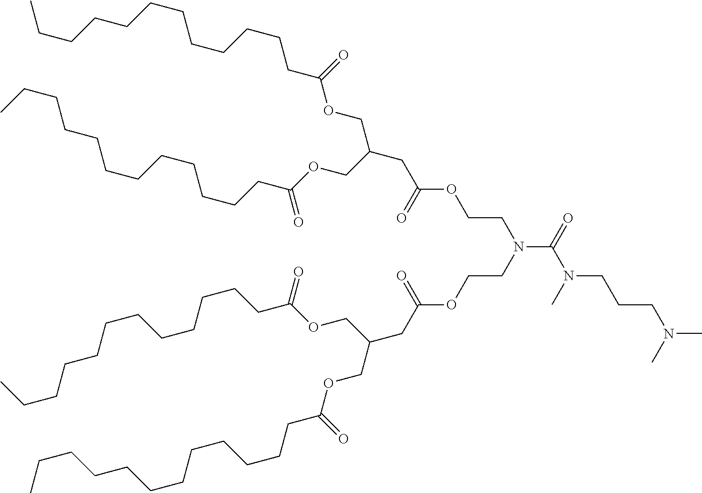 [SMILES: CCCCCCCCCCCCC(=O)OCC(COC(=O)CCCCCCCCCCCC)CC(=O)OCCN(CCOC(=O)CC(COC(=O)CCCCCCCCCCCC)COC(=O)CCCCCCCCCCCC)C(=O)N(C)CCCN(C)C] | A-15R12 | 21.31 | 9.55 |

To determine the stability of the disclosed lipids in mouse plasma lipid stock solution was prepared by dissolution of the lipid in isopropanol at the concentration of 5 mg/mL. A requisite volume of the lipid-isopropanol solution was then diluted to 100 μM concentration at a total volume of 10.0 mL with 50:50 (v/v) ethanol/water. Ten microliters of this 100 μM solution was spiked into 10.0 mL of mouse plasma (BioIVT, Lot MSE394920, CD-1 mouse, anticoagulant: sodium heparin, not filtered) that was prewarmed to 37° C. and stirred at 50 rpm with a magnetic stir bar. The starting concentration of lipids in plasma was thus 1 μM. Aliquots (50 μL) were taken after 0, 15, 30, 45, 60, and 120 minutes, transferred to microcentrifuge tubes and quenched with three volumes (150 μL) of ice cold acetonitrile/methanol (4:1). Positive control incubations utilized the same plasma, with Benfluorex (1 μM) as the substrate with Labetalol (1.0 μgl) as the in situ disappearance. The quenched solutions were vortexed, centrifuged for 5 minutes at 13,000 rpm, and supernatant (100 μL) transferred to a 96-well plate and diluted with water (200 μL, 0.1% FA). After filtration through a 0.45 μm 96-well filtering plate, the filtrates were analyzed by LC-MS (Waters AQUITY I-class UPLC system, ThermoScientific Vanquish UPLC system consisting of a binary pump, autosampler, and column compartment, Waters Xevo G2-XS QT of mass spectrometer; Phenomenex F5, 1.7 μm, 2.1×50 mm column). Mobile phase A was 0.1% formic acid in water, and mobile phase B was 0.1% formic acid in acetonitrile. The flow rate was 0.6 mL/min. Elution gradient was as follows: time, 0.5 min: 20% B; 0.5-2 min: 20-100% B; 2-4.8 min: 100% B; 4.8-5.45 min: 100-20% B. Mass spectrometry was in positive scanning mode from 600-1100 m/z. The peak of the molecular ion of the lipids was integrated in extracted ion chromatography (XIC) using Xcalibur software (Thermo Fisher). The relative peak area compared to T=0, after normalization by the peak area of the internal standard, was used as the percentage of the lipid remaining at each time point. T_{1/2} values were calculated using the first-order decay model.

All of Compounds A-2 and A-11 thru A-15 were clearly degradable in mouse plasma, comparable to benchmark 10f, but distinct from the rapidly degraded 10a and the extremely stable 10p. Even Compound A-11, the slowest degrading of the tested Compounds had a half-life of <10 hours suggesting that it could be administered several times a week without problematic accumulation. While these data only documented disappearance of the initial Compound, these lipids were designed with multiple ester linkages to promote further degradation to species that can be readily eliminated from the body without need for oxidative metabolism in the liver.

### Example 35. LNP Encapsulation of mRNA

The ability to incorporate various of the disclosed ionizable cationic lipids into LNP encapsulating mRNA was assessed using mRNA encoding the fluorescent marker mCherry.

mCherry mRNA was synthesized by T7 RNA polymerase mediated in vitro transcription (IVT) of a linearized DNA template, using full substitution of uridine with N1-Methylpseudouridine. A Cap1 structure was added to the 5′ end of the mRNA co-transcriptionally and a 3′ polyadenosine tail was encoded by the DNA template. Post IVT, mRNA was purified using a two-step chromatography process using OligoDT affinity chemistry for bulk capture and ion-pair reverse phase chemistry to remove residual impurities.

The mRNA was encapsulated in LNP using a self-assembly process in which an aqueous solution of mRNA at pH=3.5 is rapidly mixed with a solution of lipids dissolved in ethanol, then followed by stepwise phosphate and Tris buffer dilution and tangential flow filtration (TFF) purification. LNP composition in this study was: ionizable cationic lipid/distearoylphosphatidylcholine/cholesterol/DMG-PEG2000 (50:10:38.5:1.5 mol/mol) and were encapsulated at an N/P ratio (the ratio of positively-chargeable lipid amine (N=nitrogen) groups to negatively-charged nucleic acid phosphate (P) groups) at 6. LNPs were frozen at −80° C. LNP were made in which the ionizable cationic lipid was one of Compounds A-2, A-11, A-12, A-13, A-14, or A-15. The diameter of the nanoparticles was measured by dynamic light scattering using a Zetasizer Nano ZS (Malvern Instruments Ltd., Malvern, UK) instrument. Size measurement was carried out in pH 7.4 Tris buffer at 25° C. in relevant disposable capillary cells. A non-invasive back scatter system (NIBS) with a scattering angle of 173° was used for size measurements.

TABLE 7
Physical-chemical properties of the LNP

| Ionizable | Hydrodynamic | | Encapapsulation |
|---|---|---|---|
| cationic Lipid | diameter (nm) | PDI* | Efficiency (%) |
| A-12 | 104 | 0.04 | 91 |
| A-11 | 82 | 0.03 | 96 |
| A-2 | 73 | 0.13 | 99 |
| A-13 | 101 | 0.02 | 99 |
| A-15 | 97 | 0.07 | 99 |
| A-14 | 127 | 0.03 | 91 |

*polydispersity index

All of these ionizable cationic lipids formed LNP with generally acceptable size and good encapsulation efficiency. The Compound A-13 LNP showed the greatest size uniformity.

### Example 36. Transfection of HEK293F Cells

The ability of the LNP formed in the preceding Example to transfect HEK293F cells, a human embryonic kidney cell-derived cell line, with the mCherry mRNA was assessed. Viral Production Cells (Gibco Catalog number: A35347), a derivative of the HEK 293F cell line adapted to a chemically-defined, serum-free and protein-free medium (LV-MAX™ Production Medium; Gibco Catalog number: A3583401) were grown in suspension, sedimented, resuspended at about 1×10⁶ cells/mL, and 200 μL distributed to the wells of a 96-well U-bottom plate. Frozen LNP were thawed and diluted to 100 μg mRNA/mL with sterile water for injection. An appropriate volume of LNP was added to provide 0, 0.3, 0.6, or 2 μg RNA per well in duplicate and mixed by re-pipetting. The cells were then incubated for 1 hour at 37° C. in a CO₂ incubator, washed three times with phosphate buffered saline, resuspended in 400 μL of medium in a deep-well 96-well plate, and incubated at 37° C. in a CO₂ incubator on an orbital shaker at 900 RPM.

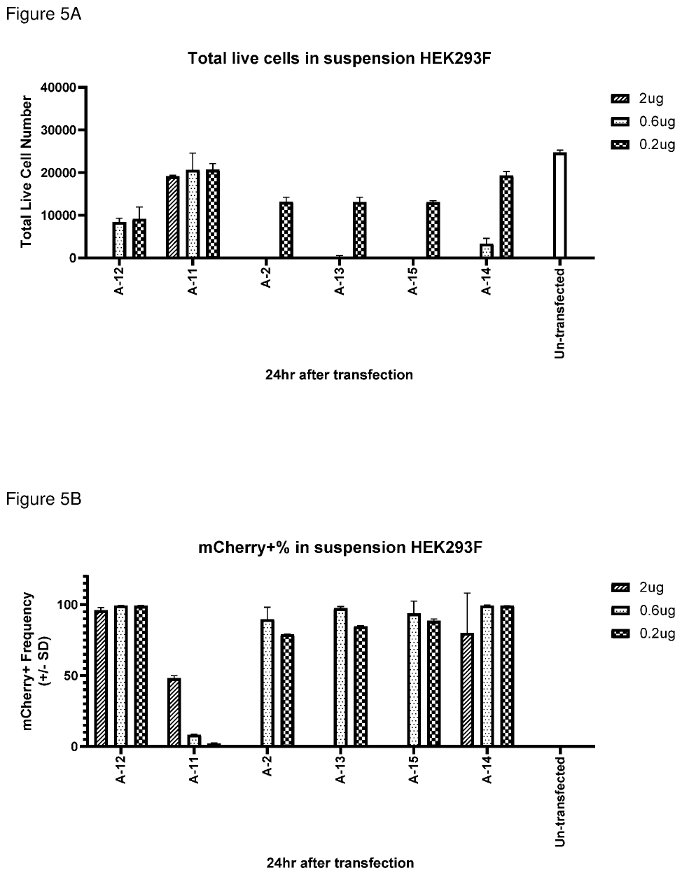

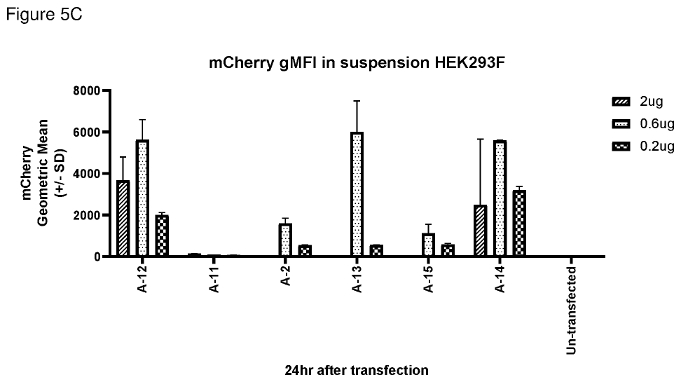

Twenty-four hours after addition of the LNP to the cells they were stained with Aqua Live/Dead (Thermo: catalog L34965) to assess cell viability. Transfection rate and expression level in the transfected cells was assessed by flow cytometry based on mCherry fluorescence. As seen in FIG. 5A, with the exception of Compound A-11, all of the LNP caused a reduction in cell number as compared to untransfected cells even at the lowest dose tested and several caused nearly complete cell killing at the higher doses. Compound A-11 caused only a small reduction in cell number even at the highest dose tested.

As seen in FIG. 5B, the all of the LNP achieved robust transfection frequency of ≥80% in the live cells, with the exception of Compound A-11 which approached only a 50% transfection rate only at the highest dose tested. Expression level was variable and without definite pattern (FIG. 12C). LNP comprising Compounds A-2, A-12, A-13, A-14, and A-15 are all more basic (more positively charged) than A-11 which correlates with their differential ability to transfect the HEK293F cells.

### Example 37. Incorporation of Ionizable Lipid into an tLNP

Each of Compounds A-2 and A-11 thru A-15 and the benchmark lipids 10a, 10f, and 10p were incorporated into a tLNP packaging an mRNA encoding the fluorescent protein mCherry and physicochemical properties measured. Results are presented in Table 6, below.

CleanCap® mCherry 5-methoxyuridine (5moU) mRNA (L-7203) was purchased from TriLink. mRNA was encapsulated in LNP using a self-assembly process in which an aqueous solution of mRNA at pH=3.5 is rapidly mixed with a solution of lipids dissolved in ethanol, then followed by stepwise phosphate and Tris buffer dilution and TFF purification. LNP composition in this study was: ionizable cationic lipid/distearoylphosphatidylcholine/cholesterol/DMG-PEG 2000/distearoylphosphatidyl ethanolamine(DSPE)-PEG2000-Maleimide (50:10:38.5:1.4:0.1 mol/mol) and were encapsulated at an N/P ratio (the ratio of positively-chargeable polymer amine (N=nitrogen) groups to negatively-charged nucleic acid phosphate (P) groups) at 6. The hydrodynamic diameter of the nanoparticles was measured by dynamic light scattering using a Zetasizer Nano ZS (Malvern Instruments Ltd., Malvern, UK) instrument.

Next, an anti-CD5 mAb was conjugated to the above LNP to generate tLNP. Purified rat anti-mouse CD5 antibody, clone 53-7.3 (BioLegend), was coupled to LNP via N-succinimidyl S-acetylthioacetate (SATA)-maleimide conjugation chemistry. Briefly, LNPs with DSPE-PEG(2000)-maleimide incorporated were formulated and stored at 4° C. on the day of conjugation. The antibody was modified with SATA (Sigma-Aldrich) to introduce sulfhydryl groups at accessible lysine residues allowing conjugation to maleimide. SATA was deprotected using 0.5 M hydroxylamine followed by removal of the unreacted components by G-25 Sephadex Quick Spin Protein columns (Roche Applied Science, Indianapolis, IN). The reactive sulfhydryl group on the antibody was then conjugated to maleimide moieties on the LNPs using thioether conjugation chemistry. Purification was performed using Sepharose CL-4B gel filtration columns (Sigma-Aldrich). tLNPs (LNPs conjugated with a targeting antibody) were frozen at −80° C. Others have conjugated antibody to free functionalized PEG-lipid and then incorporated the conjugated lipid into pre-formed LNP. However, we have found that the present procedure is more controllable and produces more consistent results.

mRNA content was determined using a Quant-iT™ RiboGreen RNA assay kit (Invitrogen™). Encapsulation efficiency was calculated by determining the unencapsulated mRNA content by measuring the fluorescence intensity (Fi) upon the addition of RiboGreen reagent to the LNP and comparing this value to the total fluorescence intensity (Ft) of the RNA content that is obtained upon lysis of the LNPs by 1% Triton X-100, where % encapsulation=(Ft−Fi)/Ft x 100).

The particle size (hydrodynamic diameter) and polydispersity index of the targeted lipid nanoparticles were determined using dynamic light scattering (DLS) on a Malvern Zetasizer Nano ZS (Malvern Instruments, Worcestershire, UK). Size measurement was carried out in pH 7.4 Tris buffer at 25° C. in relevant disposable capillary cells. A non-invasive back scatter system (NIBS) with a scattering angle of 173° was used for size measurements.

The apparent pKa of ionizable lipid in the lipid nanoparticle was determined using 6-(p-toluidino)-2-naphthalenesulfonic acid sodium salt (TNS salt, Toronto Research Chemicals, Toronto, ON, Canada). Lipid nanoparticles were diluted in 1×Dulbecco's PBS to a concentration of 1 mM total lipids. TNS salt was prepared as a 1 mg/mL stock solution in DMSO and then further diluted using distilled water to a working solution of 60 μg/mL (179 mM). Diluted lipid nanoparticle samples were further diluted to 90 μM total lipids in 165 μL of buffered solution containing 10 mM HEPES, 10 mM MES, 10 mM ammonium acetate, 130 mM NaCl, and final TNS concentration of 1.33 μg/mL (4 μM) with the pH ranging from 3.5 to 12.2. Following pipette mixing and incubation at room temperature in the dark for 15 min, fluorescence intensity was measured at room temperature in a BioTek Synergy H1 plate reader using excitation and emission wavelengths of 321 and 445 nm, respectively. The fluorescence signal was blank subtracted and plotted as a function of the pH, then analyzed using a nonlinear (Boltzmann) regression analysis with the apparent pKa determined as the pH giving rise to half maximal fluorescence intensity as calculated by the Henderson-Hasselbalch equation.

The tLNPs made in this Example are based on a reasonably conventional lipid composition, plus a functionalized PEG-lipid for conjugation of the targeting moiety and the herein disclosed ionizable cationic lipids. The conventional composition provides a good platform for assessing the contribution of the ionizable lipid to the tLNP's properties and a baseline from which to assess further optimization of the overall compositions. As seen in Table 7, all of the tLNP incorporating Compounds A-2 or A-11 thru A-15 had hydrodynamic diameters and polydispersity indices within the acceptable ranges of 50-150 nm and ≤0.2 for PDI. Encapsulation efficiency is acceptable at ≥80% although ≥85% and ≥90% are preferred. All of the tested Compounds exceeded the ≥90% threshold (although one of the benchmark lipids, 10a, did not).

TABLE 8
Physical-chemical properties of the tLNP

| Ionizable | Hydrodynamic | | Encapsulation | |
|---|---|---|---|---|
| lipid | Diameter | | Efficiency | Measured |
| in tLNP | (nm) | PDI** | (%) | pKa |
| 10a* | 86 | 0.1 | 86 | 6.09 |
| 10f* | 91 | 0.2 | 93 | 6.21 |
| 10p* | 77 | 0.2 | 96 | 6.5 |
| A-2 | 105 | 0.20 | 99 | 7.92 |
| A-11 | 93 | 0.11 | 96 | 6.65 |
| A-12 | 98 | 0.07 | 92 | 7.35 |
| A-13 | 100 | 0.10 | 99 | 7.51 |
| A-14 | 100 | 0.05 | 95 | 7.46 |
| A-15 | 136 | 0.15 | 98 | 8.37 |

*Journal of Medicinal Chemistry 63: 12992-13012, 2020
**polydispersity index

### Example 38. Targeted Transfection of T Cells In Vitro

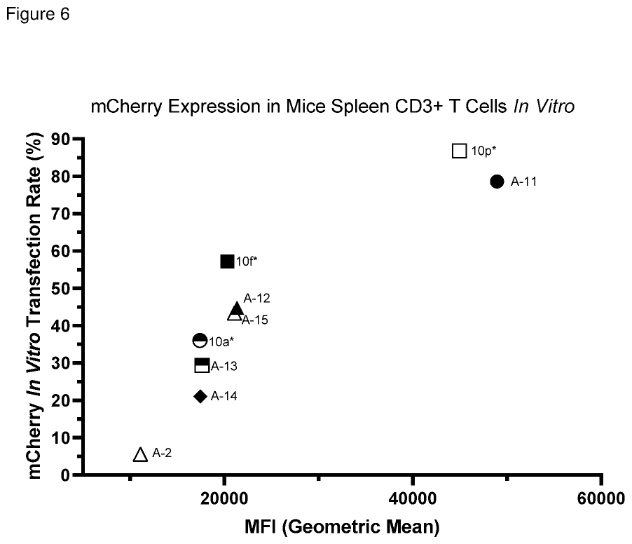

To assess the performance of tLNP described in Example 35 they were used to transfect mouse T cells in tissue culture. Mouse splenic T cells were isolated from mechanically dissociated mouse spleens using a standard T cell isolation kit (Stem Cell Technologies #19851). Isolated T cells were cultured in complete RPMI medium supplemented with murine interleukin-2 in the presence of CD3/CD28 T cell activation beads (Gibco #11453D) for 3 days. Following activation, T cells were magnetically separated from the activation beads and transferred to a 96-well plate at a concentration of 2×10⁵ cells per well in 100 μL of complete RPMI medium. tLNP formulations as described in Example 35 (above) were diluted to 100 μg/mL and 6 μL (0.6 μg) of tLNP was added to each well of cells to be tested. Cells were incubated with tLNPs at 37° C. for 1 hour before tLNPs were washed away by centrifuging the plate, removing the supernatant, and replacing with fresh medium. Transfected cells were then returned to the incubator overnight. The next day, cells were washed and resuspended in stain buffer containing fluorescently tagged antibodies against T cells markers for 30 minutes before a final wash. After washing, cells were resuspended in stain buffer and run on the Novocyte Quanteon flow cytometer to detect mCherry expression as well as murine T cell markers. Results for CD3⁺ T cells are depicted in FIG. 6.

As seen in FIG. 6, tLNP incorporating Compound A-11 and benchmark lipid 10p gave robust and comparable results with transfections rates about or over 80% and a high level of expression. Transfection with tLNP incorporating Compounds A-12 thru A-15 or the benchmark lipids 10a and 10f all resulted in similar levels of expression, less than A-11 and 10p but still substantial. Transfections rates varied from about 20% to about 60%. By comparison, the results for tLNP incorporating Compound A-2 were poor, but still positive. The superior performance of Compound A-11 among the disclosed compounds tested here correlates with it being the only one of those Compounds with a measured pKa between 6 and 7. However, performance of the other Compounds did not correlate with the size of their deviation from the preferred range for measured pKa showing that outside this range other factors dominate.

### Example 39. Targeted Transfection of T Cells In Vivo

To assess the performance of tLNP described in Example 35 they were used to transfect mouse T cells by injecting the tLNP into live mice and evaluated for their ability to generate murine T cells that express the mCherry reporter gene in vivo. All tLNP test articles were thawed at room temperature for 30 minutes and then diluted 1:2 with sterile water for injection to achieve a final dose concentration of 100 μg/mL. 100 μL (10 μg) of each test article was then injected via the tail vein into 8-week-old female C57Bl/6 mice. All treated mice were then sacrificed at 24 hours post-treatment and their spleens collected. Each spleen was then dissociated to single cell suspension and stained with antibodies to identify T cells, B cells, monocytes and non-hematopoietic cells. Stained samples were then analyzed by flow cytometry for expression of mCherry in immune cell subsets, and non-hematopoietic cells. Data analysis was performed using FlowJo (Version 10.8.1) and GraphPad Prism (9.4.1.).

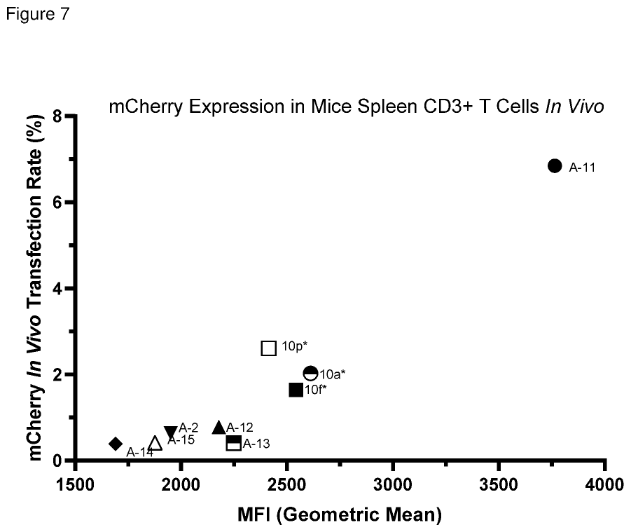

As seen in FIG. 7, both the transfection rate and level of mCherry expression are much reduced as compared to in vitro. This is expected from the lower effective dose following administration of tLNP to a live animal as compared to addition of tLNP to the well of a tissue culture plate. tLNP incorporating Compound A-11 performed markedly better than any of the others with a transfection rate of around 7% and MFI distinctly greater than that achieved with the other tLNP. tLNP incorporating the three benchmark lipids performed comparably to each other with a transfection rate of around 2% while tLNP incorporating the other tested Compounds were not clearly distinguishable from background. It was surprising that of the Compounds tested here only Compound A-11 had a measured pKa between 6 and 7, but in light of that, the poorer performance of Compounds A-2 and A-12 thru A-15 was not unexpected. That tLNP incorporating Compound A-11 performed substantially better than tLNP incorporating the benchmark lipids, which do have measured pKa's between 6 and 7 confirms that pKa is not the only determinant of performance.

### Example 40. Further Embodiments

Further embodiments are disclosed herein.

Embodiment 1. An ionizable cationic lipid having a structure of Formula 1,

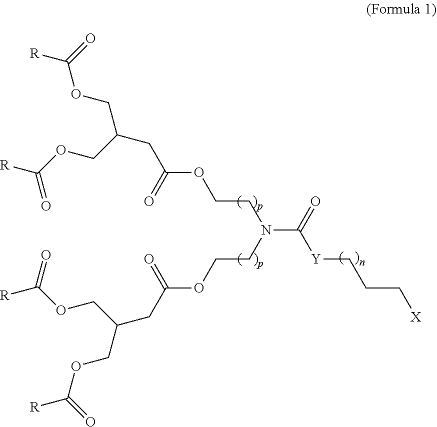

wherein Y is O, NH, N—CH₃, or CH₂,
   n is an integer from 0 to 4,
   X is

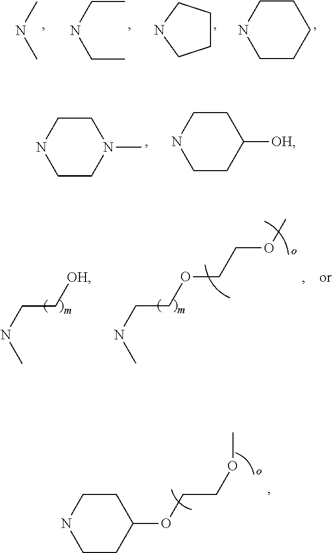

m is an integer from 1 to 3,
o is an integer from 1 to 4,
p is an integer from 1 to 4,
wherein when p=1, each R is independently C₆ to C₁₆ straight-chain alkyl; C₆ to C₁₆ branched alkyl; C₆ to C₁₆ straight-chain alkenyl; C₆ to C₁₆ branched alkenyl; C₉ to C₁₆ cycloalkyl-alkyl in which the cycloalkyl is C₃ to C₈ cycloalkyl positioned at either end or within the alkyl chain; or C₈ to C₁₈ aryl-alkyl in which the aryl is phenyl or naphthalenyl and is positioned at either end or within the alkyl chain,
wherein when p=2, each R is independently C₆ to C₁₄ straight-chain alkyl; C₆ to C₁₄ straight-chain alkenyl; C₆ to C₁₄ branched alkyl; C₆ to C₁₄ branched alkenyl; C₉ to C₁₄ cycloalkyl-alkyl in which the cycloalkyl is C₃ to C₈ cycloalkyl positioned at the either end or within the alkyl chain; C₈ to C₁₆ aryl-alkyl in which the aryl is phenyl or naphthalenyl and is positioned at either end or within the alkyl chain,
wherein when p=3, each R is independently C₆ to C₁₂ straight-chain alkyl; C₆ to C₁₂ straight-chain alkenyl; C₆ to C₁₂ branched alkyl; C₆ to C₁₂ branched alkenyl; C₉ to C₁₂ cycloalkyl-alkyl in which the cycloalkyl is C₃ to C₈ cycloalkyl positioned at either end or within the alkyl chain; C₈ to C₁₄ aryl-alkyl in which the aryl is phenyl or naphthalenyl and is positioned at the either end or within the alkyl chain, and
wherein when p=4, each R is independently C₆ to C₁₀ straight-chain alkyl; C₆ to C₁₀ straight-chain alkenyl; C₆ to C₁₀ branched alkyl; C₆ to C₁₀ branched alkenyl; C₉ to C₁₀ cycloalkyl-alkyl in which the cycloalkyl is C₃ to C₈ cycloalkyl positioned at either end or within the alkyl; C₈ to C₁₂ aryl-alky in which the aryl is phenyl or naphthalenyl and is positioned at the either end or within the alkyl chain.

Embodiment 2. An ionizable cationic lipid having a structure of Formula 2,

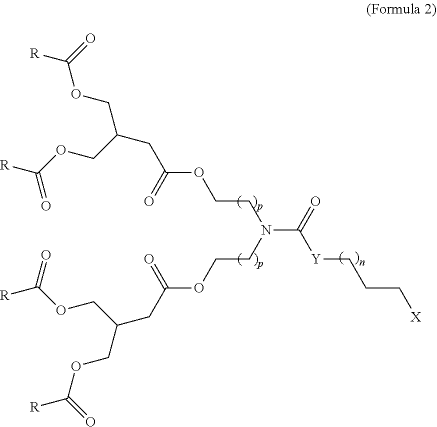

wherein Y is O, NH, N—CH₃, or CH₂,
   n is an integer from 0 to 4,
   X is

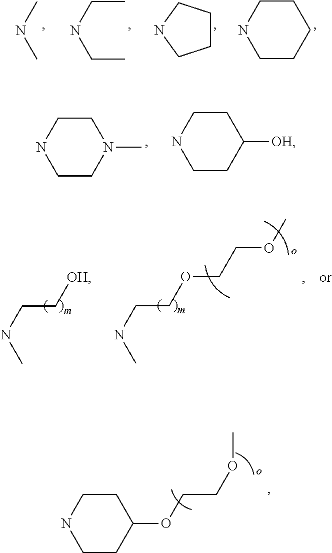

m is an integer from 1 to 3,
is an integer from 1 to 4,
p is an integer from 1 to 4,
wherein when p=1, each R is independently C₆ to C₁₆ straight-chain alkyl; C₆ to C₁₆ straight-chain alkenyl; C₆ to C₁₆ branched alkyl; C₆ to C₁₆ branched alkenyl; C₉ to C₁₆ cycloalkyl-alkyl in which the cycloalkyl is C₃ to C₈ cycloalkyl positioned at either end or within the alkyl chain; or C₈ to C₁₈ aryl-alkyl in which the aryl is phenyl or naphthalenyl and is positioned at either end or within the alkyl chain,
wherein when p=2, each R is independently C₆ to C₁₄ straight-chain alkyl; C₆ to C₁₄ straight-chain alkenyl; C₆ to C₁₄ branched alkyl; C₆ to C₁₄ branched alkenyl; C₉ to C₁₄ cycloalkyl-alkyl in which the cycloalkyl is C₃ to C cycloalkyl positioned at the either end or within the alkyl chain; or C₈ to C₁₆ aryl-alkyl in which the aryl is phenyl or naphthalenyl and is positioned at either end or within the alkyl chain,
wherein when p=3, each R is independently C₆ to C₁₂ straight-chain alkyl; C₆ to C₁₂ straight-chain alkenyl; C₆ to C₁₂ branched alkyl; branched C₆ to C₁₂ alkenyl; C₉ to C₁₂ cycloalkyl-alkyl in which the cycloalkyl is C₃ to C₈ cycloalkyl positioned at either end or within the alkyl chain; or C₈ to C₁₄ aryl-alkyl in which the aryl is phenyl or naphthalenyl and is positioned at the either end or within the alkyl chain, and
wherein when p=4, each R is independently C₆ to C₁₀ straight-chain alkyl; straight-chain C₆ to C₁₀ alkenyl; C₆ to C₁₀ branched alkyl; C₆ to C₁₀ branched alkenyl; C₉ to C₁₀ cycloalkyl-alkyl in which the cycloalkyl is C₃ to C cycloalkyl positioned at either end or within the alkyl; or C₈ to C₁₂ aryl-alky in which the aryl is phenyl or naphthalenyl and is positioned at the either end or within the alkyl chain.

Embodiment 3. An ionizable cationic lipid having a structure of Formula 3,

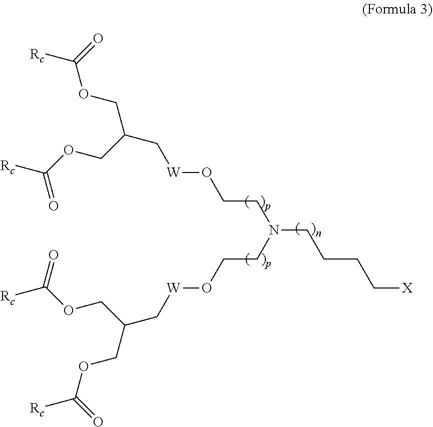

wherein W is C═O or CH₂,
   n is an integer from 0 to 4,
   X is

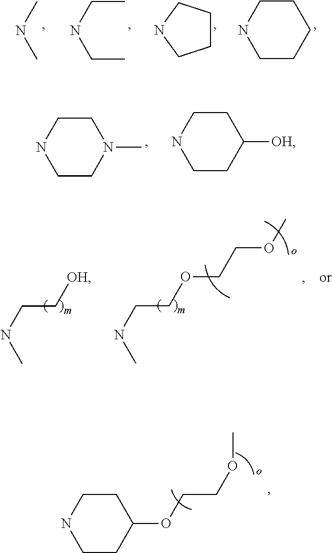

m is an integer from 1 to 3,
is an integer from 1 to 4,
p is an integer from 1 to 4,
   wherein when p=1, each R_{c} is independently C₈ to C₁₈ straight-chain alkyl; C₈ to C₁₈ straight-chain alkenyl; C₈ to C₁₈ branched alkyl; C₈ to C₁₈ branched alkenyl; C₁₁ to C₁₈ cycloalkyl-alkyl in which the cycloalkyl is C₃ to C₈ cycloalkyl positioned at either end or within the alkyl chain; or C₁₀ to C₂₀ aryl-alkyl in which the aryl is phenyl or naphthalenyl and is positioned at either end or within the alkyl chain,
   wherein when p=2, each R_{c} is independently C₈ to C₁₆ straight-chain alkyl; C₈ to C₁₆ straight-chain alkenyl; C₈ to C₁₆ branched alkyl; C₈ to C₁₆ branched alkenyl; C₁₁ to C₁₆ cycloalkyl-alkyl in which the cycloalkyl is C₃ to C₈ cycloalkyl positioned at the either end or within the alkyl chain; or C₁ to C₁₈ aryl-alkyl in which the aryl is phenyl or naphthalenyl and is positioned at either end or within the alkyl chain,
   wherein when p=3, each R_{c} is independently C₈ to C₁₄ straight-chain alkyl; C₈ to C₁₄ straight-chain alkenyl; C₈ to C₁₄ branched alkyl; C₈ to C₁₄ branched alkenyl; C₁₁ to C₁₄ cycloalkyl-alkyl in which the cycloalkyl is C₃ to C₈ cycloalkyl positioned at either end or within the alkyl chain; or C₁₀ to C₁₆ aryl-alkyl in which the aryl is phenyl or naphthalenyl and is positioned at the either end or within the alkyl chain, and
   wherein when p=4, each R_{c} is independently C₈ to C₁₂ straight-chain alkyl; C₈ to C₁₂ straight-chain alkenyl; C₈ to C₁₂ branched alkyl; C₈ to C₁₂ branched alkenyl; C₁₁ to C₁₂ cycloalkyl-alkyl in which the cycloalkyl is C₃ to C₈ cycloalkyl positioned at either end or within the alkyl; or C₁ to C₁₄ aryl-alky in which the aryl is phenyl or naphthalenyl and is positioned at the either end or within the alkyl chain.

Embodiment 4. The ionizable cationic lipid of Embodiment 1 or 2, wherein Y is O.

Embodiment 5. The ionizable cationic lipid of Embodiment 1 or 2, wherein Y is NH.

Embodiment 6. The ionizable cationic lipid of Embodiment 1 or 2, wherein Y is N—CH₃.

Embodiment 7. The ionizable cationic lipid of Embodiment 1 or 2, wherein Y is CH₂.

Embodiment 8. The ionizable cationic lipid of Embodiment 1 or 2, wherein X is

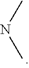

Embodiment 9. The ionizable cationic lipid of Embodiment 3, wherein W is C═O.

Embodiment 10. The ionizable cationic lipid of any one of Embodiments 1-9, comprising an R or R_{c} that is straight-chain alkyl.

Embodiment 11. The ionizable cationic lipid of any one of Embodiments 1-9, comprising an R or R_{c} that is straight-chain alkenyl.

Embodiment 12. The ionizable cationic lipid of any one of Embodiments 1-9, comprising an R or R_{c} that is branched alkyl.

Embodiment 13. The ionizable cationic lipid of any one of Embodiments 1-9, comprising an R that is branched alkenyl

Embodiment 14. The ionizable cationic lipid of any one of Embodiments 1-9, comprising an R or R_{c} that is cycloalkyl-alkyl.

Embodiment 15. The ionizable cationic lipid of any one of Embodiments 1-9, comprising an R or R_{c} that is aryl-alkyl.

Embodiment 16. The ionizable cationic lipid of any one of Embodiments 1-15, wherein each R or R_{c} group is the same.

Embodiment 17. The ionizable cationic lipid of any one of Embodiments 1-15, wherein both R or R_{c} groups stemming from a first branchpoint are the same and both R or R_{c} groups stemming from a second branchpoint are the same, but the R or R_{c} groups stemming the first branchpoint are different than the R or R_{c} groups stemming from the second branchpoint.

Embodiment 18. A lipid nanoparticle (LNP), comprising the ionizable cationic lipid of any one of Embodiments 1-17.

Embodiment 19. The LNP of Embodiment 18, further comprising one or more of a phospholipid, a sterol, a co-lipid, and a PEG-lipid, or combinations thereof.

Embodiment 20. The LNP of Embodiment 18, wherein the phospholipid comprises dioleoylphosphatidyl ethanolamine (DOPE), dimyristoylphosphatidyl choline (DMPC), distearoylphosphatidylcholine (DSPC), dimyristoylphosphatidyl glycerol (DMPG), dipalmitoyl phosphatidylcholine (DPPC), or 1,2-diarachidoyl-sn-glycero-3-phosphocholine (DAPC), or a combination thereof.

Embodiment 21. The LNP of Embodiment 18 or 19, wherein the sterol comprises cholesterol, campesterol, sitosterol, or stigmasterol, or combinations thereof.

Embodiment 22. The LNP of any one of Embodiments 18-21, wherein the co-lipid comprises cholesterol hemisuccinate (CHEMS) or a quaternary ammonium headgroup containing lipid.

Embodiment 23. The LNP of Embodiment 22, wherein the quaternary ammonium headgroup containing lipid comprises 1,2-dioleoyl-3-trimethylammonium propane (DOTAP), N-(1-(2,3-dioleyloxy)propyl)-N,N,N-trimethylammonium (DOTMA), or 3β-(N—(N′,N′-Dimethylaminoethane)carbamoyl)cholesterol (DC-Chol), or combinations thereof.

Embodiment 24. The LNP of any one of Embodiments 18-23, wherein the PEG-lipid comprises a PEG moiety of 1000-5000 Da molecular weight (MW).

Embodiment 25. The LNP of any one of Embodiments 18-24, wherein the PEG-lipid comprises fatty acids with a fatty acid chain length of C₁₄-C₁₈.

Embodiment 26. The LNP of any one of Embodiments 18-25, wherein the PEG-lipid comprises DMG-PEG2000 (1,2-dimyristoyl-rglycero-3-methoxypolyethylene glycol-2000), DPG-PEG2000 (1,2-dipalmitoyl-glycero-3-methoxypolyethylene glycol-2000), DSG-PEG2000 (1,2-distearoyl-glycero-3-methoxypolyethylene glycol-2000), DOG-PEG2000 (1,2-dioleoyl-glycero-3-methoxypolyethylene glycol-2000), DMPE-PEG200 (1,2-dimyristoyl-glycero-3-phosphoethanolamine-3-methoxypolyethylene glycol-2000), DPPE-PEG2000 (1,2-dipalmitoyl-glycero-3-phosphoethanolamine-3-methoxypolyethylene glycol-2000), DSPE-PEG2000 (1,2-distearoyl-glycero-3-phosphoethanolamine-3-methoxypolyethylene glycol-2000), DOPE-PEG2000 (1,2-dioleoyl-glycero-3-phosphoethanolamine-3-methoxypolyethylene glycol-2000), or combinations thereof.

Embodiment 27. The LNP of any one of Embodiments 18-26, wherein the PEG-lipid comprises an optically pure glycerol moiety.

Embodiment 28. The LNP of any one of Embodiments 18-27, further comprising a functionalized PEG-lipid.

Embodiment 29. The LNP of Embodiment 28, wherein the functionalized PEG-lipid has been conjugated with a binding moiety.

Embodiment 30. The LNP of Embodiment 29, wherein the binding moiety comprises an antigen-binding domain of an antibody.

Embodiment 31. The LNP of any one of Embodiments 28-30, wherein the functionalized PEG-lipid comprises fatty acids with a fatty acid chain length of C₁₆-C₁₈.

Embodiment 32. The LNP of Embodiment 31, wherein the functionalized PEG-lipid comprises a dipalmitoyl lipid or a distearoyl lipid.

Embodiment 33. The LNP of any one of Embodiments 18-32, comprising 40 to 60 mol % ionizable cationic lipid.

Embodiment 34. The LNP of any one of Embodiments 19-33, comprising 7 to 30 mol % phospholipid.

Embodiment 35. The LNP of any one of Embodiments 19-34, comprising 20 to 45 mol % sterol.

Embodiment 36. The LNP of any one of Embodiments 19-35, comprising 1 to 30 mol % co-lipid.

Embodiment 37. The LNP of any one of Embodiments 19-36, comprising 0 to 5 mol % PEG-lipid.

Embodiment 38. The LNP of any one of Embodiments 19-37, comprising 0.1 to 5 mol % functionalized PEG-lipid.

Embodiment 39. The LNP of any one of Embodiments 18-38, further comprising a nucleic acid.

Embodiment 40. The LNP of Embodiment 39, wherein the weight ratio of total lipid to nucleic acid is 10:1 to 50:1.

Embodiment 41. The LNP of Embodiment 39 or 40, comprising mRNA.

Embodiment 42. A method of delivering a nucleic acid into a cell comprising contacting the cell with the LNP of any one of Embodiments 39-41.

1. An ionizable cationic lipid having a structure of Formula 1, 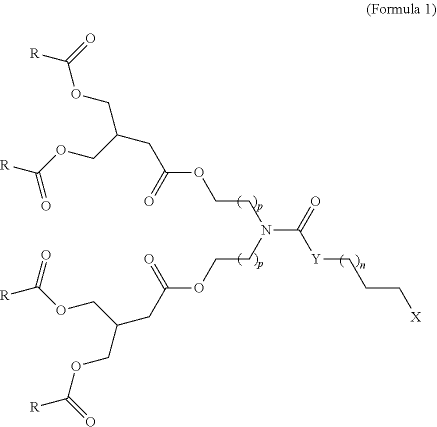
wherein Y is O, NH, N—CH₃, or CH₂,
n is an integer from 0 to 4,
X is 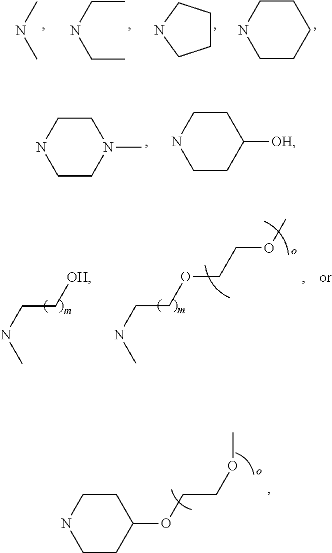
m is an integer from 1 to 3,
o is an integer from 1 to 4,
p is an integer from 1 to 4,
wherein when p=1, each R is independently C₆ to C₁₆ straight-chain alkyl; C₆ to C₁₆ branched alkyl; C₆ to C₁₆ straight-chain alkenyl; C₆ to C₁₆ branched alkenyl; C₉ to C₁₆ cycloalkyl-alkyl in which the cycloalkyl is C₃ to C₈ cycloalkyl positioned at either end or within the alkyl chain; or C₈ to C₁₈ aryl-alkyl in which the aryl is phenyl or naphthalenyl and is positioned at either end or within the alkyl chain,
wherein when p=2, each R is independently C₆ to C₁₄ straight-chain alkyl; C₆ to C₁₄ straight-chain alkenyl; C₆ to C₁₄ branched alkyl; C₆ to C₁₄ branched alkenyl; C₉ to C₁₄ cycloalkyl-alkyl in which the cycloalkyl is C₃ to C₈ cycloalkyl positioned at the either end or within the alkyl chain; C₈ to C₁₆ aryl-alkyl in which the aryl is phenyl or naphthalenyl and is positioned at either end or within the alkyl chain,
wherein when p=3, each R is independently C₆ to C₁₂ straight-chain alkyl; C₆ to C₁₂ straight-chain alkenyl; C₆ to C₁₂ branched alkyl; C₆ to C₁₂ branched alkenyl; C₉ to C₁₂ cycloalkyl-alkyl in which the cycloalkyl is C₃ to C₈ cycloalkyl positioned at either end or within the alkyl chain; C₈ to C₁₄ aryl-alkyl in which the aryl is phenyl or naphthalenyl and is positioned at the either end or within the alkyl chain, and
wherein when p=4, each R is independently C₆ to C₁₀ straight-chain alkyl; C₆ to C₁₀ straight-chain alkenyl; C₆ to C₁₀ branched alkyl; C₆ to C₁₀ branched alkenyl; C₉ to C₁₀ cycloalkyl-alkyl in which the cycloalkyl is C₃ to C₈ cycloalkyl positioned at either end or within the alkyl; C₈ to C₁₂ aryl-alky in which the aryl is phenyl or naphthalenyl and is positioned at the either end or within the alkyl chain.

2. An ionizable cationic lipid having a structure of Formula 2, 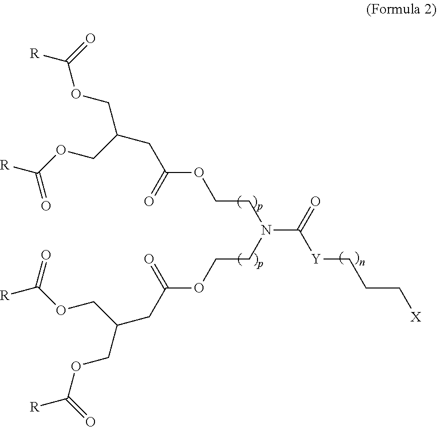
wherein Y is O, NH, N—CH₃, or CH₂,
n is an integer from 0 to 4,
X is 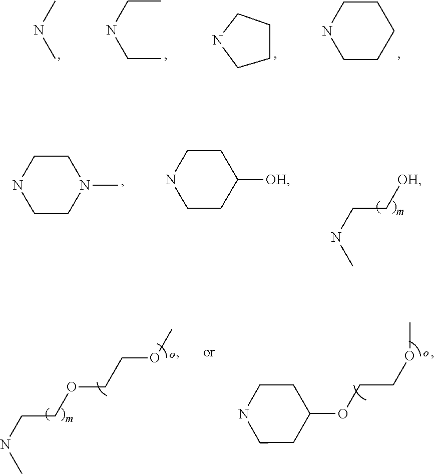
m is an integer from 1 to 3,
o is an integer from 1 to 4,
p is an integer from 1 to 4,
wherein when p=1, each R is independently C₆ to C₁₆ straight-chain alkyl; C₆ to C₁₆ straight-chain alkenyl; C₆ to C₁₆ branched alkyl; C₆ to C₁₆ branched alkenyl; C₉ to C₁₆ cycloalkyl-alkyl in which the cycloalkyl is C₃ to C₈ cycloalkyl positioned at either end or within the alkyl chain; or C₈ to C₁₈ aryl-alkyl in which the aryl is phenyl or naphthalenyl and is positioned at either end or within the alkyl chain,
wherein when p=2, each R is independently C₆ to C₁₄ straight-chain alkyl; C₆ to C₁₄ straight-chain alkenyl; C₆ to C₁₄ branched alkyl; C₆ to C₁₄ branched alkenyl; C₉ to C₁₄ cycloalkyl-alkyl in which the cycloalkyl is C₃ to C₈ cycloalkyl positioned at the either end or within the alkyl chain; or C₈ to C₁₆ aryl-alkyl in which the aryl is phenyl or naphthalenyl and is positioned at either end or within the alkyl chain,
wherein when p=3, each R is independently C₆ to C₁₂ straight-chain alkyl; C₆ to C₁₂ straight-chain alkenyl; C₆ to C₁₂ branched alkyl; branched C₆ to C₁₂ alkenyl; C₉ to C₁₂ cycloalkyl-alkyl in which the cycloalkyl is C₃ to C₈ cycloalkyl positioned at either end or within the alkyl chain; or C₈ to C₁₄ aryl-alkyl in which the aryl is phenyl or naphthalenyl and is positioned at the either end or within the alkyl chain, and
wherein when p=4, each R is independently C₆ to C₁₀ straight-chain alkyl; straight-chain C₆ to C₁₀ alkenyl; C₆ to C₁₀ branched alkyl; C₆ to C₁₀ branched alkenyl; C₉ to C₁₀ cycloalkyl-alkyl in which the cycloalkyl is C₃ to C₈ cycloalkyl positioned at either end or within the alkyl; or C₈ to C₁₂ aryl-alky in which the aryl is phenyl or naphthalenyl and is positioned at the either end or within the alkyl chain.

3. An ionizable cationic lipid having a structure of Formula 3, 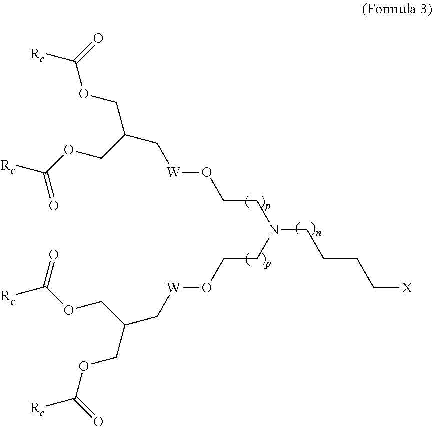
wherein W is C═O or CH₂,
n is an integer from 0 to 4,
X is 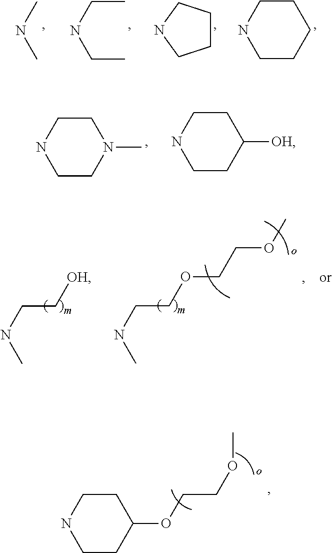
m is an integer from 1 to 3,
is an integer from 1 to 4,
p is an integer from 1 to 4,
wherein when p=1, each R_{c} is independently C₈ to C₁₈ straight-chain alkyl; C₈ to C₁₈ straight-chain alkenyl; C₈ to C₁₈ branched alkyl; C₈ to C₁₈ branched alkenyl; C₁₁ to C₁₈ cycloalkyl-alkyl in which the cycloalkyl is C₃ to C₈ cycloalkyl positioned at either end or within the alkyl chain; or C₁₀ to C₂₀ aryl-alkyl in which the aryl is phenyl or naphthalenyl and is positioned at either end or within the alkyl chain,
wherein when p=2, each R_{c} is independently C₈ to C₁₆ straight-chain alkyl; C₈ to C₁₆ straight-chain alkenyl; C₈ to C₁₆ branched alkyl; C₈ to C₁₆ branched alkenyl; C₁₁ to C₁₆ cycloalkyl-alkyl in which the cycloalkyl is C₃ to C₈ cycloalkyl positioned at the either end or within the alkyl chain; or C₁ to C₁₈ aryl-alkyl in which the aryl is phenyl or naphthalenyl and is positioned at either end or within the alkyl chain,
wherein when p=3, each R_{c} is independently C₈ to C₁₄ straight-chain alkyl; C₈ to C₁₄ straight-chain alkenyl; C₈ to C₁₄ branched alkyl; C₈ to C₁₄ branched alkenyl; C₁₁ to C₁₄ cycloalkyl-alkyl in which the cycloalkyl is C₃ to C₈ cycloalkyl positioned at either end or within the alkyl chain; or C₁₀ to C₁₆ aryl-alkyl in which the aryl is phenyl or naphthalenyl and is positioned at the either end or within the alkyl chain, and
wherein when p=4, each R_{c} is independently C₈ to C₁₂ straight-chain alkyl; C₈ to C₁₂ straight-chain alkenyl; C₈ to C₁₂ branched alkyl; C₈ to C₁₂ branched alkenyl; C₁₁ to C₁₂ cycloalkyl-alkyl in which the cycloalkyl is C₃ to C₈ cycloalkyl positioned at either end or within the alkyl; or C₁ to C₁₄ aryl-alky in which the aryl is phenyl or naphthalenyl and is positioned at the either end or within the alkyl chain.

4. The ionizable cationic lipid of claim 1, wherein Y is O.

5-7. (canceled)

8. The ionizable cationic lipid of claim 1, wherein X is

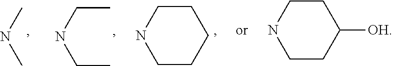

9. (canceled)

10. The ionizable cationic lipid of claim 1, comprising an R that is straight-chain alkyl.

11-15. (canceled)

16. The ionizable cationic lipid of claim 1, wherein each R group is the same.

17. The ionizable cationic lipid of claim 1, wherein both R groups stemming from a first branchpoint are the same and both R groups stemming from a second branchpoint are the same, but the R groups stemming the first branchpoint are different than the R groups stemming from the second branchpoint.

18. A lipid nanoparticle (LNP), comprising the ionizable cationic lipid of claim 1.

19. The LNP of claim 18, further comprising one or more of a phospholipid, a sterol, a co-lipid, and a PEG-lipid, or combinations thereof.

20. The LNP of claim 19, wherein
a) the phospholipid comprises dioleoylphosphatidyl ethanolamine (DOPE), dimyristoylphosphatidyl choline (DMPC), distearoylphosphatidylcholine (DSPC), dimyristoylphosphatidyl glycerol (DMPG), dipalmitoyl phosphatidylcholine (DPPC), or 1,2-diarachidoyl-sn-glycero-3-phosphocholine (DAPC), or a combination thereof,
b) the sterol comprises cholesterol, 20-hydroxycholesterol, 22-hydroxycholesterol, campesterol, sitosterol, or stigmasterol, or combinations thereof,
c) the co-lipid comprises cholesterol hemisuccinate (CHEMS) or a quaternary ammonium headgroup containing lipid,
d) the PEG-lipid comprises a PEG moiety of 1000-5000 Da molecular weight (MW), or
e) a combination of one or more of a)-d).

21-22. (canceled)

23. The LNP of claim 19, wherein the quaternary ammonium headgroup containing lipid comprises 1,2-dioleoyl-3-trimethylammonium propane (DOTAP), N-(1-(2,3-dioleyloxy)propyl)-N,N,N-trimethylammonium (DOTMA), or 3β-(N—(N′,N′-Dimethylaminoethane)carbamoyl)cholesterol (DC-Chol), or combinations thereof.

24. (canceled)

25. The LNP of claim 19, wherein the PEG-lipid comprises fatty acids with a fatty acid chain length of C₁₄-C₁₈.

26. The LNP of claim 19, wherein the PEG-lipid comprises DMG-PEG2000 (1,2-dimyristoyl-rglycero-3-methoxypolyethylene glycol-2000), DPG-PEG2000 (1,2-dipalmitoyl-glycero-3-methoxypolyethylene glycol-2000), DSG-PEG2000 (1,2-distearoyl-glycero-3-methoxypolyethylene glycol-2000), DOG-PEG2000 (1,2-dioleoyl-glycero-3-methoxypolyethylene glycol-2000), DMPE-PEG200 (1,2-dimyristoyl-glycero-3-phosphoethanolamine-3-methoxypolyethylene glycol-2000), DPPE-PEG2000 (1,2-dipalmitoyl-glycero-3-phosphoethanolamine-3-methoxypolyethylene glycol-2000), DSPE-PEG2000 (1,2-distearoyl-glycero-3-phosphoethanolamine-3-methoxypolyethylene glycol-2000), DOPE-PEG2000 (1,2-dioleoyl-glycero-3-phosphoethanolamine-3-methoxypolyethylene glycol-2000), or combinations thereof.

27. The LNP of claim 19, wherein the PEG-lipid comprises an optically pure glycerol moiety.

28. The LNP of claim 19, further comprising a functionalized PEG-lipid.

29. A LNP (tLNP), comprising
a) an ionizable cationic lipid having a structure of Formula 1, 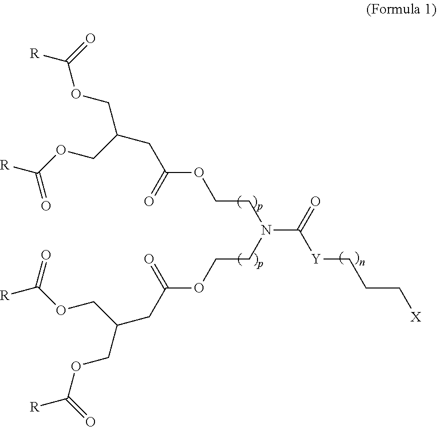
wherein Y is O, NH, N—CH₃, or CH₂,
n is an integer from 0 to 4,
X is 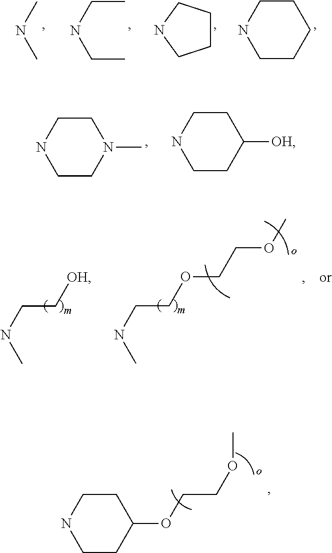
m is an integer from 1 to 3,
o is an integer from 1 to 4,
p is an integer from 1 to 4,
wherein when p=1, each R is independently C₆ to C₁₆ straight-chain alkyl; C₆ to C₁₆ branched alkyl; C₆ to C₁₆ straight-chain alkenyl; C₆ to C₁₆ branched alkenyl; C₉ to C₁₆ cycloalkyl-alkyl in which the cycloalkyl is C₃ to C₈ cycloalkyl positioned at either end or within the alkyl chain; or C₈ to C₁₈ aryl-alkyl in which the aryl is phenyl or naphthalenyl and is positioned at either end or within the alkyl chain,
wherein when p=2, each R is independently C₆ to C₁₄ straight-chain alkyl; C₆ to C₁₄ straight-chain alkenyl; C₆ to C₁₄ branched alkyl; C₆ to C₁₄ branched alkenyl; C₉ to C₁₄ cycloalkyl-alkyl in which the cycloalkyl is C₃ to C₈ cycloalkyl positioned at the either end or within the alkyl chain; C₈ to C₁₆ aryl-alkyl in which the aryl is phenyl or naphthalenyl and is positioned at either end or within the alkyl chain,
wherein when p=3, each R is independently C₆ to C₁₂ straight-chain alkyl; C₆ to C₁₂ straight-chain alkenyl; C₆ to C₁₂ branched alkyl; C₆ to C₁₂ branched alkenyl; C₉ to C₁₂ cycloalkyl-alkyl in which the cycloalkyl is C₃ to C₈ cycloalkyl positioned at either end or within the alkyl chain; C₈ to C₁₄ aryl-alkyl in which the aryl is phenyl or naphthalenyl and is positioned at the either end or within the alkyl chain, and
wherein when p=4, each R is independently C₆ to C₁₀ straight-chain alkyl; C₆ to C₁₀ straight-chain alkenyl; C₆ to C₁₀ branched alkyl; C₆ to C₁₀ branched alkenyl; C₉ to C₁₀ cycloalkyl-alkyl in which the cycloalkyl is C₃ to C₈ cycloalkyl positioned at either end or within the alkyl; C₈ to C₁₂ aryl-alky in which the aryl is phenyl or naphthalenyl and is positioned at the either end or within the alkyl chain,
b) a sterol,
c) a phospholipid,
d) a non-functionalized PEG-lipid, and
e) a functionalized PEG-lipid, wherein the functionalized PEG-lipid has been conjugated with a binding moiety.

30-32. (canceled)

33. The LNP of claim 18, comprising 40 to 60 mol % ionizable cationic lipid.

34. The LNP of claim 19, comprising
a) 7 to 30 mol % phospholipid
b) 20 to 45 mol % sterol,
c) 1 to 30 mol % co-lipid,
d) 0 to 5 mol % PEG-lipid,
e) 0.1 to 5 mol % functionalized PEG-lipid, or
f) a combination of one or more of a)-e).

35-37. (canceled)

38. The LNP of claim 19, comprising 0.1 to 5 mol % functionalized PEG-lipid.

39. The LNP of claim 18, further comprising a nucleic acid.

40. The LNP of claim 39, wherein the weight ratio of total lipid to nucleic acid is 10:1 to 50:1.

41. The LNP of claim 39, wherein the nucleic acid comprises an mRNA.

42. A method of delivering a nucleic acid into a cell comprising contacting the cell with the LNP of claim 39.
